(19) United States
(12) Patent Application Publication
(10) Pub. No.: US 2016/0024595 A1
(43) Pub. Date: Jan. 28, 2016

(54) CHARACTERIZATION OF MELANOMA USING A MOLECULAR SIGNATURE

(71) Applicant: DermTech International, La Jolla, CA (US)

(72) Inventor: John P. ALSOBROOK, II, Corrales, NM (US)

(21) Appl. No.: 14/806,453

(22) Filed: Jul. 22, 2015

Related U.S. Application Data
(60) Provisional application No. 62/027,730, filed on Jul. 22, 2014.

(51) Int. Cl. C12Q 1/68; A61N 5/10; A61B 10/02

(52) U.S. Cl. CPC C12Q 1/6886; A61B 10/02; A61N 5/10; C12Q 2600/16; C12Q 2600/158; C12Q 2600/112

(57) ABSTRACT

The present disclosure provides methods for characterizing melanoma in a subject by analyzing genes or gene expression products obtained from the subject. The present disclosure provides methods for distinguishing melanoma in situ from invasive melanoma. The disclosure includes non-invasive methods for obtaining genes or gene expression products from a pigmented skin lesion of a subject.

### CROSS-REFERENCE

This application claims the benefit of U.S. Provisional Application No. 62/027,730 filed Jul. 22, 2014, which is incorporated herein in its entirety.

### BACKGROUND OF THE INVENTION

Melanoma is a serious form of skin cancer in humans. It arises from the pigment cells (melanocytes), usually found in the skin. The incidence of melanoma is increasing at the fastest rate of all cancers in the United States with a lifetime risk of 1 in 68. Although melanoma accounts for only 4% of all dermatologic cancers, it is responsible for 80% of all deaths from skin cancers.

### SUMMARY OF THE INVENTION

The prognosis for a subject with melanoma is largely dependent on the stage of melanoma in a subject. The ability to cure melanoma in its earliest skin stage, melanoma in situ (MIS), is virtually 100% if the melanoma is adequately surgically excised. If the melanoma is caught in a later stage, where it has invaded to a depth of 4 mm or more, the ten-year survival rate is less than 50%. If the melanoma is not detected until it has spread to distant parts of the body, the prognosis is dismal, with only 7-9% of patients surviving 5 years, with the median survival time being 8-9 months. Tragically, the long-term “cure” rate for late stage melanoma is only 1-2%.

Melanoma treatment options vary with the progression of the disease. For example, for MIS, a biopsy with a narrow surgical margin, usually about 0.5 cm, is typically performed at the site of the melanoma skin lesion. However, for invasive melanomas or melanomas suspected of being invasive, a more extensive biopsy may be performed with wide local surgical margins of about 1 cm to 3 cm, where the resulting defect may necessitate closure by skin grafting. In addition, a biopsy of the sentinel lymph nodes may be performed on invasive melanoma samples to assess the presence of metastasis in the regional lymph nodes. Additional methods of treatment for invasive melanomas involve regional lymphadenectomy, adjuvant therapy with high-dose interferon alpha-2b, immunotherapy (e.g., checkpoint inhibitors such as PD-1 and PD-L1 inhibitors), signal transduction inhibitors, and even chemotherapy. There are potential adverse effects for each of these treatments, particularly for invasive melanoma treatments. A sentinel lymph node biopsy or lymphadenectomy may cause lymphedema, lymphangiosarcoma, and/or seroma. It is therefore important to distinguish between melanoma in situ and invasive melanoma so that an appropriate treatment approach is applied. Identification of a patient with invasive melanoma will allow for a more aggressive treatment approach that has a higher chance of treating and/or removing the melanoma than if a simple biopsy were performed. Similarly, identification of a patient with melanoma in situ will allow for a less invasive treatment option with fewer complications than those performed to treat invasive melanomas.

In various aspects, the methods described herein allow for a skin sample identified as comprising melanoma to be characterized as either melanoma in situ or invasive melanoma. In various embodiments, the methods employ non-invasive skin sampling techniques that allow for a skin sample to be obtained and characterized as melanoma in situ or invasive melanoma. In some aspects, the methods involve determining expression levels of one or more gene expression products of a gene classifier in a sample identified as having melanoma cells. In some embodiments, the combinations of genes in the gene classifiers described herein are particularly useful for distinguishing melanoma in situ from invasive melanoma, and the determination of their expression values for said distinguishing does not preclude use and/or analysis of these genes from other applications that do not involve melanoma, a cancer, or a skin disease state.

Provided herein, in various aspects, are methods, devices and systems for characterizing melanoma in a subject. According to one feature of the subject matter described herein, melanoma in situ is distinguished from invasive melanoma in a subject having melanoma by determining an expression profile comprising 1, 2, 3, 4, 5 or 6 target genes selected from WFDC3, SLC16A6, DUSP4, PPAP2A, NDUFAF4 and BCL2A1 by assaying the gene expression products in the biological sample comprising a plurality of gene expression products from the subject, and classifying the subject as having melanoma in situ or invasive melanoma based on the expression profile, thereby distinguishing melanoma in situ from invasive melanoma. In some embodiments, the biological sample is obtained from the subject. In some embodiments, the method further comprises determining an expression profile for at least one additional target gene selected from one or more genes listed in Table 1 in the biological sample. In some embodiments, the biological sample is a tissue, blood, serum, plasma or urine sample. In some embodiments the biological sample is a skin sample. In some embodiments, the skin sample is a skin lesion. In some embodiments, the skin sample comprises one or more melanoma cells.

In some embodiments, a skin sample is obtained by applying an adhesive tape to a target area of skin in a manner sufficient to adhere the skin sample to the adhesive tape and removing the adhesive tape from the skin in a manner sufficient to retain the adhered skin sample to the adhesive tape. In some embodiments, the adhesive tape is part of a skin sample collection kit. In some embodiments, the adhesive tape does not contain latex. In some embodiments, the skin sample collection kit further comprises a sample collector and an instructions for use sheet. In some embodiments, the adhesive tape comprises a rubber adhesive on a polyurethane film. In some embodiments, about one to about ten adhesive tapes or about one to about ten applications of an adhesive tape are applied to and removed from the skin. In some embodiments, the gene expression products of the biological sample are isolated from the adhesive tape.

In some embodiments, the gene expression products are nucleic acid molecules. In some embodiments, the nucleic acid molecules are RNA molecules. In some embodiments, the method further comprises amplifying the nucleic acid molecules. In some embodiments, the method further comprises generating cDNA from the RNA molecules. In some embodiments, the nucleic acid molecules are amplified by polymerase chain reaction (PCR). In some embodiments, the amplification products are quantified using real-time quantitative PCR.

In some embodiments, assaying the gene expression products in the biological sample to determine the gene expression profile comprises hybridization of one or more probes to the gene expression products; and provided that the one or more probes comprise nucleic acid sequences specific and complementary to one or more of the genes of the gene classifier. In some embodiments, the one or more probes are configured to hybridize to 1, 2, 3, 4, 5 or 6 gene expression products expressed from the genes in the gene classifier.

In some embodiments, the gene expression products are RNA molecules and the method further comprises generating cDNA molecules from the RNA molecules. In some embodiments, assaying the gene expression products in the biological sample comprises quantifying the cDNA molecules. In some embodiments, the cDNA molecules are quantified using real time quantitative PCR to determine the gene expression profile. In some embodiments, a cDNA molecule is quantitated by hybridization of a detectably labeled probe specific for the cDNA molecule to the cDNA molecule. In some cases, the probe is labeled with a fluorophore and a quencher. In some embodiments, assaying the gene expression products in the biological sample to determine the gene expression profile comprises hybridization of one or more probes to the cDNA molecules; and provided that the one or more probes comprise nucleic acid sequences specific and complementary to one or more of the genes of the gene classifier. In some embodiments, the one or more probes are configured to hybridize to 1, 2, 3, 4, 5 or 6 cDNA molecules transcribed from gene expression products expressed from the genes in the gene classifier.

In some embodiments, assaying the gene expression products in the biological sample to determine the expression profile comprises microarray analysis, digital gene expression or a direct sequencing method. In some embodiments, assaying the gene expression products in the biological sample comprises applying the gene expression products or amplicon products thereof to a microarray. In some embodiments, assaying the gene expression products in the biological sample comprises applying the cDNA molecules or amplification products thereof to a microarray. In some embodiments, the microarray is a DNA microarray comprising one or more probes. In some embodiments, the one or more probes are configured to hybridize to 1, 2, 3, 4, 5, or 6 nucleic acid molecules comprising nucleic acid sequences, or complementary nucleic acid sequences of the genes in the gene classifier.

In some embodiments, the expression profile is determined by microarray analysis, digital gene expression or a direct sequencing method. In some embodiments, the gene expression product or an amplicon product thereof is applied to a microarray. In some embodiments, the microarray is a DNA microarray comprising probes. In some embodiments, the microarray comprises probes configured to bind to 1, 2, 3, 4, 5, or 6 nucleic acid molecules selected from the genes WFDC3, SLC16A6, DUSP4, PPAP2A, NDUFAF4 and BCL2A1. In some embodiments, the gene expression products are protein molecules. In some embodiments, the expression profile is determined by a binding assay. In some embodiments, the binding assay comprises at least one (e.g., 1, 2, 3, 4, 5 or 6) antibody configured to detect at least one protein expressed from the genes WFDC3, SLC16A6, DUSP4, PPAP2A, NDUFAF4 and/or BCL2A1.

In some embodiments, classifying the subject as having melanoma in situ or invasive melanoma is accomplished by comparing the expression profile of the one or more genes of the gene classifier in the biological sample to a gene expression profile of the one or more genes of the gene classifier in at least one reference sample, provided that the at least one reference sample is selected from an individual with melanoma in situ or invasive melanoma.

In some embodiments, classifying the subject as having melanoma in situ or invasive melanoma comprises performing a hierarchical cluster analysis that merges the expression profile of the biological sample with similar expression profiles from either a first subset of reference samples or a second subset of reference samples; provided that the first subset of reference samples comprise melanoma in situ and the second subset of reference samples comprise invasive melanoma.

In some embodiments, classifying the subject as having melanoma in situ or invasive melanoma comprises (a) calculating a score based on the gene expression profile of the biological sample; and (b) correlating the score of the biological sample to a threshold value; provided that the threshold value is a number between (i) one or more melanoma in situ score values calculated from one or more corresponding gene expression profiles from one or more reference samples comprising melanoma in situ, and (ii) one or more invasive melanoma score values calculated from one or more corresponding gene expression profiles from one or more reference samples comprising invasive melanoma. In some embodiments, the corresponding gene expression profiles comprise expression values from one or more of the genes in the gene classifier comprising WFDC3, SLC16A6, DUSP4, PPAP2A, NDUFAF4 and BCL2A1. In some embodiments, the average value of the one or more melanoma in situ score values is a number lower than the threshold value and the average value of the one or more invasive melanoma score values is a number higher than the threshold value. In some cases, if the score of the biological sample is below the threshold value, the biological sample comprises melanoma in situ. In some cases, if the score of the biological sample is above the threshold value, the biological sample comprises invasive melanoma. In some embodiments, the average value of the one or more melanoma in situ score values is a number greater than the threshold value and the average value of the one or more invasive melanoma score values is a number lower than the threshold value. In some cases, if the score of the biological sample is above the threshold value, the biological sample comprises melanoma in situ. In some embodiments, the score of the biological sample is below the threshold value the biological sample comprises invasive melanoma.

In some cases, a subject having melanoma in situ refers to a biological sample of the subject comprising melanoma in situ. In some cases, a subject having invasive melanoma refers to a biological sample of the subject comprising invasive melanoma. As a non-limiting example, a skin lesion is a biological sample.

In some embodiments, nucleic acid expression products expressed from 1, 2, 3, 4, 5 or 6 of the genes in the gene classifier comprising WFDC3, SLC16A6, DUSP4, PPAP2A, NDUFAF4 and BCL2A1, are detected for their presence or absence in a biological sample suspected of comprising the nucleic acid expression products expressed from the 1, 2, 3, 4, 5 or 6 genes in the gene classifier. In some embodiments, the presence or absence of nucleic acid expression products from 1, 2, 3, 4, 5 or 6 of the genes in the gene classifier is indicative of the presence of melanoma in the biological sample. In some embodiments, the presence or absence of nucleic acid expression products from 1, 2, 3, 4, 5 or 6 of the genes in the gene classifier is indicative of melanoma in situ. In some embodiments, the presence or absence of nucleic acid expression products from 1, 2, 3, 4, 5 or 6 of the genes in the gene classifier is indicative of invasive melanoma. In some embodiments, detecting the presence or absence of a nucleic acid expressed from a gene in the gene classifier comprises applying one or more probes to the biological sample, wherein each probe is capable of hybridizing to 1, 2, 3, 4, 5 or 6 nucleic acids expressed from one or more genes in the gene classifier. For example, each probe is configured to bind to each nucleic acid expression product of a gene in the gene classifier. In some cases, the biological sample is tissue, blood, serum, plasma or urine sample. In some cases, the biological sample comprises a skin sample. In some cases, the biological sample comprises a skin lesion. In some cases, the biological sample comprises one or more melanoma cells.

In some embodiments, expression levels of nucleic acids expressed from 1, 2, 3, 4, 5 or 6 of the genes in the gene classifier comprising WFDC3, SLC16A6, DUSP4, PPAP2A, NDUFAF4 and BCL2A1, are determined in a biological sample suspected of or known to comprise nucleic acid expression products of the gene classifier. In some embodiments, determining the expression level of a nucleic acid expressed from a gene in the gene classifier comprises applying one or more probes to the biological sample, wherein each probe is capable of hybridizing to 1, 2, 3, 4, 5 or 6 nucleic acids expressed from one or more genes in the gene classifier. For example, each probe is configured to bind to each nucleic acid expression product of a gene in the gene classifier. In some cases, determining the expression level of a nucleic acid comprises performing PCR on the biological sample to amplify the nucleic acids. In some cases, determining the expression level of a nucleic acid comprises performing quantitative PCR on the biological sample. In some cases, determining the expression level of a nucleic acid comprises performing real-time quantitative PCR on the biological sample. In some embodiments, the expression level of one or more nucleic acid expression products is compared a known expression level of the one or more nucleic acid expression products in a reference sample. In some cases, the reference sample comprises a sample having melanoma in situ. In some cases, the reference sample comprises a sample having invasive melanoma.

In some embodiments, the method further comprises determining a treatment regimen based on the classification of melanoma as melanoma in situ or invasive melanoma. In some embodiments, classifying the subject as having melanoma in situ or invasive melanoma is accomplished by comparing the expression profile of the at least one target gene from the biological sample to a gene expression profile of the at least one gene from at least one reference sample, provided that the at least one reference sample is selected from an individual with melanoma in situ or invasive melanoma.

In some embodiments, the method further comprises determining the stage of a melanoma in a subject, provided that a subject is classified as having invasive melanoma. In some embodiments, the stage of melanoma is 1, 2, 3 or 4. In some embodiments, the method further comprises administering a specific treatment for melanoma to the subject if the melanoma is classified as melanoma in situ. In some embodiments, the method further comprises administering a specific treatment for melanoma if the melanoma is classified as invasive melanoma.

In some embodiments, the biological sample is a first biological sample obtained from the subject at a first time point. In some embodiments, the method further comprises determining an expression profile of the one or more genes of the gene classifier by assaying the gene expression products from a second biological sample obtained from the subject having melanoma at a second time point; comparing the expression profile from the first time point to the second time point; and assessing the progression of melanoma in a subject; provided that a change in the expression profile at the second time point relative to the first time point is indicative of the progression of melanoma. In some embodiments, the method further comprises administering a treatment if the melanoma progressed from the first time point to the second time point. In some embodiments, the treatment is a biopsy.

In some embodiments, the method further comprises determining an expression profile of the at least one target gene selected from the genes WFDC3, SLC16A6, DUSP4, PPAP2A, NDUFAF4 and BCL2A1 by assaying the gene expression products in the biological sample from the at least one target gene at a second time point; comparing the expression profile from a first time point to the second time point; provided that a change in the expression profile at the second time point relative to the first time point is indicative of the progression of melanoma; and assessing the progression of melanoma in a subject. In some embodiments, the method further comprises administering a specific treatment if the melanoma progressed from the first time point to the second time point. In some embodiments, the method further comprises performing a biopsy if the melanoma progressed from the first time point to the second time point.

According to one aspect of the subject matter, the response of a subject with melanoma to treatment is detected by treating a subject for melanoma, providing a biological sample comprising a plurality of gene expression products from the subject having melanoma; and determining an expression profile of 1, 2, 3, 4, 5, or 6 target genes selected from the genes WFDC3, SLC16A6, DUSP4, PPAP2A, NDUFAF4 and BCL2A1 by assaying the gene expression products in the biological sample from the at least one target genes at a time point; provided that the expression profile is informative of a response to a treatment, thereby detecting a response of a subject with melanoma to a melanoma treatment. In some embodiments, the method further comprises obtaining the biological sample from the subject. In some embodiments, the method further comprises determining an expression profile for at least one additional target gene selected from one or more genes listed in Table 1 in the biological sample. In some embodiments, the biological sample is a tissue, blood, serum, plasma or urine sample. In some embodiments, the biological sample is a skin sample. In some embodiments, the skin sample is a skin lesion. In some embodiments, the skin sample comprises one or more melanoma cells.

In some embodiments, the skin sample is obtained by applying an adhesive tape to a target area of skin in a manner sufficient to adhere a skin sample to the adhesive tape and removing the adhesive tape from the skin in a manner sufficient to retain the adhered skin sample to the adhesive tape. In some embodiments, the adhesive tape is part of a skin sample collection kit. In some embodiments, the adhesive tape does not contain latex. In some embodiments, the skin sample collection kit further comprises a sample collector and a instructions for use sheet. In some embodiments, the adhesive tape comprises a rubber adhesive on a polyurethane film. In some embodiments, about one to about ten adhesive tapes or about one to about ten applications of an adhesive tape are applied to and removed from the skin. In some embodiments, the gene expression products of the biological sample are isolated from the adhesive tape.

In some embodiments, the gene expression products are nucleic acid molecules. In some embodiments, the nucleic acid molecules are RNA molecules. In some embodiments, the method further comprises amplifying the nucleic acid molecules. In some embodiments, the method further comprises generating cDNA from the RNA molecules. In some embodiments, the nucleic acid molecules are amplified by polymerase chain reaction (PCR). In some embodiments, PCR is real-time quantitative PCR. In some embodiments, the expression profile is determined by microarray analysis, digital gene expression or a direct sequencing method. In some embodiments, the gene expression product or an amplicon product thereof is applied to a microarray. In some embodiments, the microarray is a DNA microarray comprising probes. In some embodiments, the probes are configured to bind to 1, 2, 3, 4, 5, or 6 nucleic acid molecules selected from the genes WFDC3, SLC16A6, DUSP4, PPAP2A, NDUFAF4 and BCL2A1. In some embodiments, the gene expression products are protein molecules. In some embodiments, the expression profile is determined by a binding assay. In some embodiments, the binding assay utilizes at least one antibody configured to detect at least one protein expressed from the genes WFDC3, SLC16A6, DUSP4, PPAP2A, NDUFAF4 and BCL2A1.

In some embodiments, the gene expression products are RNA molecules and the method further comprises generating cDNA molecules from the RNA molecules. In some embodiments, assaying the gene expression products in the biological sample comprises quantifying the cDNA molecules. In some embodiments, the cDNA molecules are quantified using real time quantitative PCR to determine the gene expression profile. In some embodiments, a cDNA molecule is quantitated by hybridization of a detectably labeled probe specific for the cDNA molecule to the cDNA molecule. In some cases, the probe is labeled with a fluorophore and a quencher. In some embodiments, assaying the gene expression products in the biological sample to determine the gene expression profile comprises hybridization of one or more probes to the cDNA molecules; and provided that the one or more probes comprise nucleic acid sequences specific and complementary to one or more of the genes of the gene classifier. In some embodiments, the one or more probes are configured to hybridize to 1, 2, 3, 4, 5 or 6 cDNA molecules transcribed from gene expression products expressed from the genes in the gene classifier.

In some embodiments, assaying the gene expression products in the biological sample to determine the expression profile comprises microarray analysis, digital gene expression or a direct sequencing method. In some embodiments, assaying the gene expression products in the biological sample comprises applying the gene expression products or amplicon products thereof to a microarray. In some embodiments, assaying the gene expression products in the biological sample comprises applying the cDNA molecules or amplification products thereof to a microarray. In some embodiments, the microarray is a DNA microarray comprising one or more probes. In some embodiments, the one or more probes are configured to hybridize to 1, 2, 3, 4, 5, or 6 nucleic acid molecules comprising nucleic acid sequences, or complementary nucleic acid sequences of the genes in the gene classifier.

In some embodiments, the expression profile is determined by microarray analysis, digital gene expression or a direct sequencing method. In some embodiments, the gene expression product or an amplicon product thereof is applied to a microarray. In some embodiments, the microarray is a DNA microarray comprising probes. In some embodiments, the microarray comprises probes configured to bind to 1, 2, 3, 4, 5, or 6 nucleic acid molecules selected from the genes WFDC3, SLC16A6, DUSP4, PPAP2A, NDUFAF4 and BCL2A1. In some embodiments, the gene expression products are protein molecules. In some embodiments, the expression profile is determined by a binding assay. In some embodiments, the binding assay comprises at least one (e.g., 1, 2, 3, 4, 5 or 6) antibody configured to detect at least one protein expressed from the genes WFDC3, SLC16A6, DUSP4, PPAP2A, NDUFAF4 and/or BCL2A1.

In some embodiments, the method further comprises modifying a treatment regimen based on the response of a subject with melanoma to melanoma treatment. In some embodiments, the method further comprises comprising comparing the expression profile of the at least one target gene from the biological sample to a gene expression profile of the at least one target gene from at least one reference sample, provided that the at least one reference sample is selected from an individual with melanoma. In some embodiments, the method further comprises determining the stage of a melanoma in a subject, provided that a subject is classified as having melanoma.

According to one aspect of the subject matter, a kit for characterizing melanoma in a subject is provided, comprising at least one primer or probe for the detection of at least 1, 2, 3, 4, 5, or 6 gene expression products selected from the genes WFDC3, SLC16A6, DUSP4, PPAP2A, NDUFAF4 and BCL2A1 in a biological sample. In some embodiments, the gene expression products are nucleic acid molecules. In some embodiments, the nucleic acid molecules are RNA molecules. In some embodiments, the kit further comprises RNA isolation reagents. In some embodiments, the gene expression products are protein molecules. In some embodiments, the probes are antibodies. In some embodiments, the antibodies are configured to bind to 1, 2, 3, 4, 5, or 6 gene expression products selected from the genes WFDC3, SLC16A6, DUSP4, PPAP2A, NDUFAF4 and BCL2A1. In some embodiments, the kit further comprises a skin sample collection device. In some embodiments, the skin sample collection device is non-invasive. In some embodiments, the skin sample collection device is an adhesive tape. In some embodiments, the adhesive tape is a rubber-based, pliable adhesive tape. In some embodiments, the kit further comprises a sample collector. In some embodiments, the kit further comprises an instructions for use sheet.

In another aspect of the disclosure, provided herein are methods for the detection and characterization of melanoma in a subject suspected of having melanoma. In one aspect, the method comprises (a) providing a first biological sample from the subject suspected of having melanoma; (b) detecting the presence of one or more nucleic acids expressed from a first gene classifier comprising LINC00518, CMIP and PRAME; (c) if the one or more expressed nucleic acids of the first gene classifier are present, determining an expression value of the one or more expressed nucleic acids; (d) comparing the expression value of the one or more expressed nucleic acids in the sample to an expression value of the one or more expressed nucleic acids in a first reference sample, wherein the first reference sample comprises one or more expressed nucleic acids of the first gene classifier in a sample comprising melanoma or in a sample that does not comprise melanoma, or both samples that comprise and do not comprise melanoma; (e) identifying the subject as having melanoma or not having melanoma based off of the comparison; (f) if the subject has melanoma, providing a second biological sample from the subject; (g) detecting the presence of one or more nucleic acids expressed from a second gene classifier comprising WFDC3, SLC16A6, DUSP4, PPAP2A, NDUFAF4 and BCL2A1; (h) if the one or more nucleic acids expressed from the second gene classifier are present, determining an expression value of the one or more expressed nucleic acids; (i) comparing the expression values of the one or more expressed nucleic acids from the second gene classifier to a second reference, wherein the second reference comprises expression values of the genes in the second gene classifier for a sample that has melanoma in situ, a sample that has invasive melanoma, or samples that have melanoma in situ and invasive melanoma; and (j) identifying the subject as having melanoma in situ or invasive melanoma based off the comparison. In some embodiments, the first biological sample and the second biological sample are derived from the same sample from the subject. In some embodiments, the first reference is the same as the second reference.

In some embodiments, nucleic acid expression products expressed from 1, 2, 3, 4, 5 or 6 of the genes in the gene classifier comprising WFDC3, SLC16A6, DUSP4, PPAP2A, NDUFAF4 and BCL2A1, are detected for their presence or absence in a biological sample suspected of comprising the nucleic acid expression products expressed from the 1, 2, 3, 4, 5 or 6 genes in the gene classifier. In some embodiments, the presence or absence of nucleic acid expression products from 1, 2, 3, 4, 5 or 6 of the genes in the gene classifier is indicative of the presence of melanoma in the biological sample. In some embodiments, the presence or absence of nucleic acid expression products from 1, 2, 3, 4, 5 or 6 of the genes in the gene classifier is indicative of melanoma in situ. In some embodiments, the presence or absence of nucleic acid expression products from 1, 2, 3, 4, 5 or 6 of the genes in the gene classifier is indicative of invasive melanoma. In some embodiments, detecting the presence or absence of a nucleic acid expressed from a gene in the gene classifier comprises applying one or more probes to the biological sample, wherein each probe is capable of hybridizing to 1, 2, 3, 4, 5 or 6 nucleic acids expressed from one or more genes in the gene classifier. For example, each probe is configured to bind to each nucleic acid expression product of a gene in the gene classifier. In some cases, the biological sample is tissue, blood, serum, plasma or urine sample. In some cases, the biological sample comprises a skin sample. In some cases, the biological sample comprises a skin lesion. In some cases, the biological sample comprises one or more melanoma cells.

In some embodiments, expression levels of nucleic acids expressed from 1, 2, 3, 4, 5 or 6 of the genes in the gene classifier comprising WFDC3, SLC16A6, DUSP4, PPAP2A, NDUFAF4 and BCL2A1, are determined in a biological sample suspected of or known to comprise nucleic acid expression products of the gene classifier. In some embodiments, determining the expression level of a nucleic acid expressed from a gene in the gene classifier comprises applying one or more probes to the biological sample, wherein each probe is capable of hybridizing to 1, 2, 3, 4, 5 or 6 nucleic acids expressed from one or more genes in the gene classifier. For example, each probe is configured to bind to each nucleic acid expression product of a gene in the gene classifier. In some cases, determining the expression level of a nucleic acid comprises performing PCR on the biological sample to amplify the nucleic acids. In some cases, determining the expression level of a nucleic acid comprises performing quantitative PCR on the biological sample. In some cases, determining the expression level of a nucleic acid comprises performing real-time quantitative PCR on the biological sample. In some embodiments, the expression level of one or more nucleic acid expression products is compared a known expression level of the one or more nucleic acid expression products in a reference sample. In some cases, the reference sample comprises a sample having melanoma in situ. In some cases, the reference sample comprises a sample having invasive melanoma.

According to another aspect of the subject matter, provided herein is a method for distinguishing melanoma in situ from invasive melanoma in a subject, the method comprising: providing a biological sample comprising a plurality of nucleic acid expression products from a lesion of a subject identified to comprise melanoma; determining an expression profile of one or more genes in a gene signature comprising WFDC3, SLC16A6, DUSP4, PPAP2A, NDUFAF4 and BCL2A1 by assaying the nucleic acid expression products in the biological sample; calculating a score of the biological sample using the determined expression profile; correlating the score of the biological sample to a threshold value; provided that the threshold value is a number between (a) one or more melanoma in situ score values calculated from one or more corresponding gene expression profiles of one or more samples comprising melanoma in situ, and (b) one or more invasive melanoma score values calculated from one or more corresponding gene expression profiles of one or more samples comprising invasive melanoma; and classifying the lesion as comprising melanoma in situ or invasive melanoma based on the correlation between the score of the biological sample and the threshold value. In some embodiments, the score of the biological sample is a score calculated by logistical regression and the score is a regression score. In some embodiments, the melanoma in situ score is a score calculated by logistical regression, and provided that the invasive melanoma score is a score calculated by logistical regression. In some embodiments, the nucleic acid expression products are RNA expression products. In some embodiments, assaying the nucleic acid expression profile comprises generating complementary (cDNA) products from the RNA expression products expressed from the one or more genes in the gene signature, and performing real-time quantitative PCR to amplify and quantify the cDNA products to determine expression levels of the one or more genes in the gene signature.

In some embodiments, at least about 80%, 85%, 90%, 95%, 99% or 100% of the one or more melanoma in situ score values are less than the threshold value and at least about 80%, 85%, 90%, 99% or 100% of the one or more invasive melanoma score values are greater than the threshold value. In some embodiments, the average of the one or more melanoma in situ score values is less than the threshold value and the average of the one or more invasive melanoma score values is greater than the threshold value. In some embodiments, if the score of the biological sample is less than the threshold value, the lesion is classified as comprising melanoma in situ, and provided that if the score of the biological sample is greater than the threshold value, the lesion is classified as comprising invasive melanoma.

In some embodiments, at least about 80%, 85%, 90%, 95%, 99% or 100% of the one or more melanoma in situ score values are greater than the threshold value and at least about 80%, 85%, 90%, 99% or 100% of the one or more invasive melanoma score values are less than the threshold value. In some embodiments, the average of the one or more melanoma in situ score values is greater than the threshold value and the average of the one or more invasive melanoma score values is less than the threshold value. In some embodiments, if the score of the biological sample is greater than the threshold value, the lesion is classified as comprising melanoma in situ, and provided that if the score of the biological sample is less than the threshold value, the lesion is classified as comprising invasive melanoma.

In some embodiments, the lesion of the subject is identified to comprise melanoma by histopathological analysis of a biopsy of the lesion.

In some embodiments, the lesion of the subject is identified to comprise melanoma by a method comprising: providing a specimen from the lesion of the subject; provided that the specimen comprises nucleic acid expression products expressed from one or more genes in a melanoma diagnostic signature comprising LINC00518, CMIP, and PRAME; determining expression levels of the nucleic acid expression products in the specimen; comparing the expression levels of the nucleic acid expression products in the specimen to expression levels of the corresponding nucleic acid expression products in one or more reference samples; provided that one of the one or more reference samples comprises melanoma or does not comprise melanoma, thereby identifying the lesion as comprising melanoma or not comprising melanoma. In some embodiments, at least one of the one or more reference samples does not comprise melanoma. In some embodiments, if the expression level of nucleic acid expression products expressed by LINC00518 in the specimen is greater than the expression level of nucleic acid expression products expression by LINC00518 in the at least one of the one or more reference samples that does not comprise melanoma, the lesion is identified as comprising melanoma. In some embodiments, if the expression level of nucleic acid expression products expressed by PRAME in the specimen is greater than the expression level of nucleic acid expression products expression by PRAME in the at least one of the one or more reference samples that does not comprise melanoma, the lesion is identified as comprising melanoma. In some embodiments, if the expression level of nucleic acid expression products expressed by CMIP in the specimen is less than the expression level of nucleic acid expression products expression by CMIP in the at least one of the one or more reference samples that does not comprise melanoma, the lesion is identified as comprising melanoma. In some embodiments, the specimen is the biological sample.

### BRIEF DESCRIPTION OF THE DRAWINGS

The novel features of the disclosure are set forth with particularity in the appended claims. A better understanding of the features and advantages of the present disclosure will be obtained by reference to the following detailed description that sets forth illustrative embodiments, in which the principles of the disclosure are utilized, and the accompanying drawings of which:

FIG. 1 exemplifies the frequency of occurrences of each gene in different 6-gene combination classifiers having AUC-ROC values greater than 0.9.

FIG. 2 exemplifies the AUC-ROC values of gene combinations as genes are removed one-by-one from a 20-gene classifier. The y-axis is the AUC-ROC and the x-axis is the number of genes in the classifier. An inflection is seen at N=7 genes, where performance remains similar regardless of the number of additional genes in the classifier.

FIG. 3 provides a ROC curve for a 6-gene classifier comprising WFDC3, SLC16A6, DUSP4, PPAP2A, NDUFAF4, BCL2A1 for distinguishing between melanoma in situ and invasive melanoma.

FIG. 4 exemplifies the case distribution of classifier scores for 77 skin lesion samples. The x-axis is the case number. The y-axis represents the regression score based on expression levels of a 6-gene classifier. Melanomas in situ are plotted on the left as closed triangles (▴). Invasive melanomas are plotted on the right as closed circles ().

### DETAILED DESCRIPTION OF THE INVENTION

Provided herein are methods and kits for determining a molecular signature from a subject to characterize melanoma. For example, characterizing melanoma as melanoma in situ or invasive melanoma. In some instances, the molecular signature from a subject is compared to one or more reference molecular signatures. In some instances, the reference molecular signature corresponds to a reference sample with a melanoma characteristic, for example, melanoma in situ or invasive melanoma. In another aspect, the methods are useful for monitoring the progression of melanoma in a subject. In another aspect, the methods are useful for determining the effectiveness of melanoma treatment in a subject. For example, by comparing the molecular signature prior to treatment with the molecular signature after treatment. In another aspect, the molecular signature is useful for determining an appropriate treatment regimen for melanoma. Thus, the methods of the disclosure are useful for providing a means for practicing personalized medicine, wherein treatment is tailored to a subject based on the particular characteristics of the disease in the subject.

In some embodiments, the molecular signature of a subject is a gene expression profile. In some embodiments, the molecular signature of a subject is a gene sequence profile. In some embodiments, the molecular signature is a protein sequence profile.

Also provided herein is a non-invasive method for collecting skin cells comprising cellular materials useful for obtaining a molecular signature of a subject. Cellular materials useful for obtaining a molecular signature in a subject include genes or gene expression products. Cellular materials obtained from the non-invasive method provided herein include, but are not limited to, cells, nucleic acids, polypeptides, lipids, small molecules, and carbohydrates. The non-invasive method involves a tape stripping procedure that permits a direct quantitative and qualitative assessment of a molecular signature. Although tape-harvested genes and gene expression products are shown to be comparable in quality and utility to recovering such molecules by biopsy, the non-invasive method provides information regarding cells of the outermost layers of the skin that may not be obtained using biopsy samples. The non-invasive method is also far less traumatic than a biopsy.

The non-invasive method captures cellular material from a pigmented skin lesion that is either positive for melanoma or suspicious of being melanoma. Genes or gene expression products obtained from skin cells collected by the non-invasive method are analyzed in order to characterize the nature of the lesion (e.g., melanoma in situ or invasive melanoma). In one embodiment, isolated and/or purified RNA from a collected skin sample is reverse transcribed into cDNA for amplification by PCR to enrich for target genes. The expression levels of these target genes are quantified by quantitative PCR (e.g., real-time quantitative PCR) to generate a gene expression profile. In another embodiment, one or more proteins expressed from target genes are isolated and/or purified from a collected skin sample and then quantitated to obtain a gene expression profile. In another embodiment, one or more genes are isolated and/or purified from a collected skin sample and then sequenced to obtain a gene sequence profile. In another embodiment, one or more proteins expressed from target genes are isolated and/or purified from a collected skin sample. The amino acid sequence of the protein is identified to obtain a protein sequence profile.

The methods provided herein for the determination of a molecular signature are not limited to isolated and/or purified genes or gene expression products obtained from the tape stripping method. In some embodiments, the genes and/or gene expression products are analyzed for a molecular signature while remaining bound to the tape. The methods provided herein are not limited to non-invasive techniques for obtaining skin samples containing cellular material. For example, but not by limitation, one of skill in the art would know other techniques for obtaining a skin sample such as scraping of the skin, biopsy, suction, blowing and other techniques. As described herein, non-invasive tape stripping is an illustrative example for obtaining a skin sample. The methods provided herein are not limited to cellular material obtained from skin samples. Cellular material may be obtained from blood, serum, plasma, hair, sweat, tears, urine, or any combination thereof.

### Certain Terminology

As used herein, the term “pigmented skin lesion” refers to a change in the color or texture in an area of skin. As such, “pigmented skin lesions suspected of being melanoma” are pigmented skin lesions with characteristics of malignant melanoma, which are well known to those of skill in the art, such as dermatologists and oncologists. Such lesions are sometimes raised and can have a color that is different from the color of normal skin of an individual (e.g., brown, black, red, or blue). Lesions suspected of being melanoma sometimes include a mixture of colors, are often asymmetrical, can change in appearance over time, and may bleed. A pigmented skin lesion suspected of being melanoma may be a mole or nevus. Melanoma lesions are usually, but not always, larger than 6 mm in diameter. Melanoma includes superficial spreading melanoma, nodular melanoma, acral lentiginous melanoma, and lentigo maligna melanoma. Melanoma can occur on skin that has been overexposed to the sun. Therefore, in one embodiment the skin sample is taken from an area of skin that has been overexposed to the sun.

The term “melanoma” includes, but is not limited to, melanomas, metastatic melanomas, melanomas derived from either melanocytes or melanocyte related nevus cells, melanocarcinomas, melanoepitheliomas, melanosarcomas, melanoma in situ, superficial spreading melanoma, nodular melanoma, lentigo maligna melanoma, acral lentiginous melanoma, invasive melanoma or familial atypical mole and melanoma (FAMM) syndrome.

The term “melanoma in situ”, in various instances, refers to the first stage of melanoma (stage 0), wherein the melanoma cells are only present in the outer layer of the skin (epidermis).

As used herein, the term “gene” refers to a linear sequence of nucleotides along a segment of DNA that provides the coded instructions for synthesis of RNA, which can lead to the expression of hereditary character. In some instances, a gene provides coded instructions for translation into protein. In some instances, a gene provides coded instructions for the synthesis of non-coding RNA, which does not lead to translation into protein. In some instances, the term “target gene” or “biomarker” refers to a gene whose expression level is different depending on a characteristic of melanoma. For example, a characteristic of melanoma is a stage of melanoma such as melanoma in situ or invasive melanoma. The term “target gene” or “biomarker” may also refer to a gene whose expression level in a sample is different depending on if the sample has or does not have melanoma. The term “target gene” or “biomarker” may also refer to a gene whose expression level in a sample correlates to a melanoma characteristic. Therefore, expression of a melanoma target gene of the disclosure is related to, or indicative of, melanoma. Many statistical techniques are known in the art, which can be used to determine whether a statistically significant difference or correlation in expression is observed at a high (e.g., 90% or 95%) confidence level. As such, the expression levels of these genes are related to and can characterize melanoma.

As used herein, “gene expression product” means any product expressed by a gene, including nucleic acids or polypeptides. In some embodiments, a gene product is a transcribed nucleic acid, such as RNA. In some embodiments, the RNA is a coding RNA, e.g. a messenger RNA (mRNA). In some embodiments, the RNA is a non-coding RNA. In some embodiments, the non-coding RNA is a transfer RNA (tRNA), ribosomal RNA (rRNA), snoRNA, microRNA, siRNA, snRNA, exRNA, piRNA and long ncRNA. In some embodiments, a gene product is a protein that is translated from mRNA or other nucleic acid.

As used herein, the term “protein” comprises at least one polypeptide comprising amino acids residues bound together by peptide bonds.

As used herein, the term “sample” or “biological sample” refers to any preparation containing cellular material derived from a subject. In some embodiments, a sample is a skin sample derived from a subject. In some embodiments, a sample obtained using the non-invasive tape stripping method described herein is used to collect cellular material including, but not limited to, polynucleotides, polypeptides, and/or lipids, for the methods provided herein. In some embodiments, samples for the methods provided herein are taken from a pigmented skin lesion that is suspected of being the result of a disease or a pathological state, such as melanoma. In some embodiments, samples for the methods provided herein are taken from a pigmented skin lesion having melanoma. In some embodiments, samples are taken of the skin surface of the suspicious lesion using non-invasive skin sampling methods described herein. In some instances, a sample is a tissue, hair, blood, serum, plasma, sweat, tear, urine, or other sample comprising cellular material.

As used herein, the term “skin” refers to the outer protective covering of the body, consisting of the corium and the epidermis, and is understood to include sweat and sebaceous glands, as well as hair follicle structures. As used herein, the term “cutaneous” refers generally to attributes of the skin, as appropriate to the context in which they are used. In some embodiments, the skin is mammalian skin. In some embodiments, the skin is human skin. In some embodiments, a target area of skin is an area of skin comprising a pigmented skin lesion.

As used herein, the term “treatment” refers to any systematic plan or course for treating a disease or cancer in a subject, particularly melanoma.

As used herein, the term “ROC” refers to a receiver operating characteristic (ROC) or ROC curve. A ROC curve is a graphical plot illustrative of the performance of a classifier system as a discrimination threshold is varied. A ROC curve is a plot of the fraction of true positives out of the total actual positives versus the fraction of false positives out of the total actual negatives at various threshold settings. The area under the ROC curve is the AUC, for area under curve. The AUC is a probability that a classifier will rank a randomly chosen positive instance higher than a randomly chosen negative one.

### Molecular Signature

The molecular signatures presented herein are indicative of a characteristic of melanoma. In some embodiments, the molecular signature differentiates between melanoma in situ and invasive melanoma. In some embodiments, the molecular signature is indicative of a stage of melanoma, for example stage 0, 1, 2, 3, or 4. In some embodiments, a molecular signature is obtained from a subject suspicious for having melanoma; wherein the molecular signature is indicative of a subject having or not having melanoma. In some embodiments, the molecular signature is indicative of the progression of melanoma with a given treatment; wherein the molecular signature of a subject is obtained at different time points throughout treatment. In some embodiments, the molecular signature is indicative of the putative effectiveness of a given treatment for melanoma; wherein a subject with a specific molecular signature is more or less likely to respond positively to a given treatment.

The molecular signatures presented herein are determined by analyzing genes and/or gene expression products obtained from cellular material. In some embodiments, the cellular material is obtained from a pigmented skin lesion suspicious of melanoma. In some embodiments, the cellular material is obtained from a pigmented skin lesion positive for melanoma. In some instances, the cellular material is tissue obtained by tape stripping a skin sample of the superficial epidermis overlying the lesion. In one feature, in addition to tape striping, a standard biopsy of the same lesion is also performed, along with accompanying histology and diagnosis. The cellular material is obtained from blood, serum, plasma, sweat, hair, tears, urine, and other techniques known by one of skill in the art.

In another aspect, the methods of the present disclosure involve in situ analysis of a pigmented skin lesion for characterization thereof. For in situ methods, cellular material does not need to be isolated or purified from the subject prior to analysis. In one embodiment, detectably labeled probes are contacted with cellular material or a tissue of a subject for detection of expressed RNA to characterize the pigmented skin lesion. In one embodiment, detectably labeled probes are contacted with cellular material or a tissue of a subject for detection of expressed polypeptides to characterize the pigmented skin lesion. The detection of the labeled probes may be visual or may be performed and analyzed using a software program or module on a computer.

The molecular signature presented herein comprises one or more of the following genes or gene expression products WFDC3, SLC16A6, DUSP4, PPAP2A, NDUFAF4 and BCL2A1. In some instances, the molecular signature comprises 2, 3, 4, 5, or 6 of the following genes or gene expression products WFDC3, SLC16A6, DUSP4, PPAP2A, NDUFAF4 and BCL2A1. In some embodiments, the molecular signature comprises WFDC3 and SLC16A6. In some embodiments, the molecular signature comprises WFDC3 and DUSP4. In some embodiments, the molecular signature comprises WFDC3 and PPAP2A. In some embodiments, the molecular signature comprises WFDC3 and NDUFAF4. In some embodiments, the molecular signature comprises WFDC3 and BCL2A1. In some embodiments, the molecular signature comprises SLC16A6 and DUSP4. In some embodiments, the molecular signature comprises SLC16A6 and PPAP2A. In some embodiments, the molecular signature comprises SLC16A6 and NDUFAF4. In some embodiments, the molecular signature comprises SLC16A6 and BCL2A1. In some embodiments, the molecular signature comprises DUSP4 and PPAP2A. In some embodiments, the molecular signature comprises DUSP4 and NDUFAF4. In some embodiments, the molecular signature comprises DUSP4 and BCL2A1. In some embodiments, the molecular signature comprises PPAP2A and NDUFAF4. In some embodiments, the molecular signature comprises PPAP2A and BCL2A1. In some embodiments, the molecular signature comprises NDUFAF4 and BCL2A1. In some embodiments, the molecular signature further comprises one or more genes or gene expression products selected from DMXL2, SMG1, SLC2A3, VEGFA, BEX4, SH3BP5, RAB27A, FEM1A, CYTH3, IRGQ, EFNA5, NAMPT, PNPT1, and HNRNPH3. In some instances, the molecular signature further comprises 1, 2, 3, 4, 5, 6, 7, 8, 9, 10, 11, 12, 13, or 14 genes or gene expression products selected from DMXL2, SMG1, SLC2A3, VEGFA, BEX4, SH3BP5, RAB27A, FEM1A, CYTH3, IRGQ, EFNA5, NAMPT, PNPT1, and HNRNPH3.

In some embodiments, the molecular signature comprises one or more of the following genes or gene expression products WFDC3, SLC16A6, DUSP4, PPAP2A, NDUFAF4, BCL2A1, DMXL2, SMG1, SLC2A3, VEGFA, BEX4, SH3BP5, RAB27A, FEM1A, CYTH3, IRGQ, EFNA5, NAMPT, PNPT1, and HNRNPH3. In some embodiments, the molecular signature comprises 1, 2, 3, 4, 5, 6, 7, 8, 9, 10, 11, 12, 13, 14, 15, 16, 17, 18, 19 or 20 of the following genes or gene expression products WFDC3, SLC16A6, DUSP4, PPAP2A, NDUFAF4, BCL2A1, DMXL2, SMG1, SLC2A3, VEGFA, BEX4, SH3BP5, RAB27A, FEM1A, CYTH3, IRGQ, EFNA5, NAMPT, PNPT1, and HNRNPH3 as listed in Table 1.

TABLE 1
20 Gene Molecular Signature.

| GENE | CHROMOSOMAL | |
|---|---|---|
| SYMBOL | LOCATION | GENE NAME |
| WFDC3 | 20q13.12 | WAP FOUR-DISULFIDE CORE DOMAIN 3 |
| DMXL2 | 15q21.2 | DMX-LIKE 2 |
| SLC16A6 | 22q12.3 | SOLUTE CARRIER FAMILY 16 (MONOCARBOXYLIC ACID |
| | | TRANSPORTER), MEMBER 6 |
| SMG1 | 16p12.3 | SMG1, C. ELEGANS, HOMOLOG OF |
| SLC2A3 | 12p13.31 | SOLUTE CARRIER FAMILY 2 (FACILITATED GLUCOSE |
| | | TRANSPORTER), MEMBER 3 |
| VEGFA | 6p21.1 | VASCULAR ENDOTHELIAL GROWTH FACTOR A |
| BEX4 | Xq22.1 | BEX FAMILY MEMBER 4 (Brain-expresse, X-linked, 4) |
| SH3BP5 | 3p25.1 | SH3 DOMAIN-BINDING PROTEIN 5 |
| NDUFAF4 | 6q16.1 | NADH DEHYDROGENASE 1 ALPHA SUBCOMPLEX, ASSEMBLY |
| | | FACTOR 4 |
| RAB27A | 15q21.3 | RAS-ASSOCIATED PROTEIN RAB27A |
| FEM1A | 19p13.3 | FEM1, C. ELEGANS, HOMOLOG OF, A |
| PPAP2A | 5q11 | PHOSPHATIDIC ACID PHOSPHATASE TYPE 2A |
| CYTH3 | 7p22.1 | CYTOHESIN 3 |
| IRGQ | 19q13.31 | IMMUNITY-RELATED GTPASE FAMILY, Q |
| DUSP4 | 8p12 | DUAL-SPECIFICITY PHOSPHATASE 4 |
| EFNA5 | 5q21 | EPHRIN A5 |
| NAMPT | 7q22.3 | NICOTINAMIDE PHOSPHORIBOSYLTRANSFERASE |
| BCL2A1 | 15q25.1 | BCL2-RELATED PROTEIN A1 |
| PNPT1 | 2p16.1 | POLYRIBONUCLEOTIDE NUCLEOTIDYLTRANSFERASE 1 |
| HNRNPH3 | 10q21.3 | HETEROGENEOUS NUCLEAR RIBONUCLEOPROTEIN H3 |

In some embodiments, in order to provide a basis for the characterization of a pigmented skin lesion using the molecular signature from the pigmented skin lesion, a reference database is established. The reference database contains a number of reference profiles from skin samples of subjects with known pigmented skin lesion characteristics, such as normal (i.e., non-melanoma) and various melanoma states (e.g., melanoma, melanoma in situ, invasive melanoma, stage 1 melanoma, stage 2 melanoma, stage 3 melanoma, and/or stage 4 melanoma). In some embodiments, the molecular signature of a subject is compared with the reference database containing the reference profiles. If the molecular profile of the subject matches best with the profile of a particular pigmented skin lesion characteristic in the database, the subject is diagnosed as having such pigmented skin lesion characteristic. Various computer systems and software can be utilized for implementing the analytical methods of this disclosure and are apparent to one of skill in the art. Exemplary software programs include, but are not limited to, Cluster & TreeView (Stanford), GeneCluster (MIT/Whitehead Institute), Array Explorer (SpotFire Inc) and GeneSpring (Silicon Genetics Inc). For computer systems and software, see also U.S. Pat. No. 6,203,987, incorporated herein by reference). In some embodiments, a regression score for the molecular signature of the subject is calculated using logistical regression. In some embodiments, the regression score of the subject is compared to a regression score of one or more reference values. In some cases, a reference value is a regression score calculated from a molecular signature of a skin lesion comprising melanoma in situ. In some cases, a reference value is a regression score calculated from a molecular signature of a skin lesion comprising invasive melanoma. In some embodiments, characterization of a skin lesion by comparing the molecular signature from the skin lesion to one or more reference molecular signatures comprises using a computation method such as hierarchical clustering or random forest.

In one aspect, the disclosure provides a method of distinguishing melanoma in situ from invasive melanoma. In one embodiment, the method includes determining and analyzing a molecular signature of a subject comprising 1, 2, 3, 4, 5, or 6 of the following genes or gene expression products WFDC3, SLC16A6, DUSP4, PPAP2A, NDUFAF4 and BCL2A1. In some embodiments, the method includes further analyzing 1, 2, 3, 4, 5, 6, 7, 8, 9, 10, 11, 12, 13, or 14 genes or gene expression products selected from DMXL2, SMG1, SLC2A3, VEGFA, BEX4, SH3BP5, RAB27A, FEM1A, CYTH3, IRGQ, EFNA5, NAMPT, PNPT1, and HNRNPH3. In some embodiments, the method includes analyzing 1, 2, 3, 4, 5, 6, 7, 8, 9, 10, 11, 12, 13, 14, 15, 16, 17, 18, 19, or 20 of the following genes or gene expression products WFDC3, SLC16A6, DUSP4, PPAP2A, NDUFAF4, BCL2A1, DMXL2, SMG1, SLC2A3, VEGFA, BEX4, SH3BP5, RAB27A, FEM1A, CYTH3, IRGQ, EFNA5, NAMPT, PNPT1, and HNRNPH3. In some embodiments, analyzing comprises comparing the expression levels of the subject's molecular signature to one or more reference molecular signatures, wherein the one or more reference molecular signatures correspond to samples having melanoma in situ, samples having invasive melanoma, or a combination samples having melanoma in situ and samples having invasive melanoma; and wherein if the subject's molecular signature best matches to a reference molecular signature, the subject is determined to have the disease state that correlates to the matched reference sample. In some embodiments, analyzing comprises comparing an expression level of one or more gene products of the subject's molecular signature to an expression level of the one or more gene products of a reference molecular signature, wherein an increase in expression level of the one or more gene products of the subject's molecular signature is indicative of a disease state. In some embodiments, analyzing comprises comparing an expression level of one or more gene products of the subject's molecular signature to an expression level of the one or more gene products of a reference molecular signature, wherein a decrease in expression level of the one or more gene products of the subject's molecular signature is indicative of a disease state. In some cases, the disease state is melanoma in situ. In some cases, the disease state is invasive melanoma. In some embodiments, a regression score is calculated based on expression levels of the gene expression products of the molecular signature. In some embodiments, the regression score is compared to one or more reference regression scores, wherein a regression score is calculated from expression levels of the gene expression products of the molecular signature in a reference sample, and wherein the reference sample corresponds to a sample having melanoma in situ or invasive melanoma. In some cases, a sample having melanoma in situ refers to a skin sample comprising melanoma in situ. In some cases, a sample having invasive melanoma refers to a skin sample comprising invasive melanoma.

In another aspect, the disclosure provides a method of distinguishing melanoma from solar lentigo and/or dysplastic nevi and/or normal pigmented skin in a subject. In one embodiment, the method includes determining and analyzing a molecular signature of a subject comprising 1, 2, 3, 4, 5, or 6 of the following genes or gene expression products WFDC3, SLC16A6, DUSP4, PPAP2A, NDUFAF4 and BCL2A1. In some embodiments, the method includes further analyzing 1, 2, 3, 4, 5, 6, 7, 8, 9, 10, 11, 12, 13, or 14 genes or gene expression products selected from DMXL2, SMG1, SLC2A3, VEGFA, BEX4, SH3BP5, RAB27A, FEM1A, CYTH3, IRGQ, EFNA5, NAMPT, PNPT1, and HNRNPH3. In some embodiments, the method includes analyzing 1, 2, 3, 4, 5, 6, 7, 8, 9, 10, 11, 12, 13, 14, 15, 16, 17, 18, 19, or 20 of the following genes or gene expression products WFDC3, SLC16A6, DUSP4, PPAP2A, NDUFAF4, BCL2A1, DMXL2, SMG1, SLC2A3, VEGFA, BEX4, SH3BP5, RAB27A, FEM1A, CYTH3, IRGQ, EFNA5, NAMPT, PNPT1, and HNRNPH3. In some embodiments, analyzing comprises comparing the expression levels of the subject's molecular signature to one or more reference molecular signatures, wherein the one or more reference molecular signatures correspond to samples having melanoma, samples not having melanoma (e.g., solar lentigo and/or dysplastic nevi), or a combination thereof; and wherein if the subject's molecular signature best matches to a reference molecular signature, the subject is determined to have or not have the disease state that correlates to the matched reference sample. In some embodiments, analyzing comprises comparing an expression level of one or more gene products of the subject's molecular signature to an expression level of the one or more gene products of a reference molecular signature, wherein an increase in expression level of the one or more gene products of the subject's molecular signature is indicative of melanoma. In some embodiments, analyzing comprises comparing an expression level of one or more gene products of the subject's molecular signature to an expression level of the one or more gene products of a reference molecular signature, wherein a decrease in expression level of the one or more gene products of the subject's molecular signature is indicative of melanoma.

In another aspect, the disclosure provides a method of staging melanoma as stage 0, 1, 2, 3, or 4. In one embodiment, the method includes determining and analyzing a molecular signature of a subject comprising 1, 2, 3, 4, 5, or 6 of the following genes or gene expression products WFDC3, SLC16A6, DUSP4, PPAP2A, NDUFAF4 and BCL2A1. In some embodiments, the method includes further analyzing 1, 2, 3, 4, 5, 6, 7, 8, 9, 10, 11, 12, 13, or 14 genes or gene expression products selected from DMXL2, SMG1, SLC2A3, VEGFA, BEX4, SH3BP5, RAB27A, FEM1A, CYTH3, IRGQ, EFNA5, NAMPT, PNPT1, and HNRNPH3. In some embodiments, the method includes analyzing 1, 2, 3, 4, 5, 6, 7, 8, 9, 10, 11, 12, 13, 14, 15, 16, 17, 18, 19, or 20 of the following genes or gene expression products WFDC3, SLC16A6, DUSP4, PPAP2A, NDUFAF4, BCL2A1, DMXL2, SMG1, SLC2A3, VEGFA, BEX4, SH3BP5, RAB27A, FEM1A, CYTH3, IRGQ, EFNA5, NAMPT, PNPT1, and HNRNPH3. In some embodiments, analyzing comprises comparing the expression levels of the subject's molecular signature to one or more reference molecular signatures, wherein the one or more reference molecular signatures correspond to samples having melanoma stage 0, samples having melanoma stage 1, samples having melanoma stage 2, samples having melanoma stage 3, samples having melanoma stage 4, or a combination thereof; and wherein if the subject's molecular signature best matches to a reference molecular signature, the subject is determined to have the disease state that correlates to the matched reference sample. In some embodiments, analyzing comprises comparing an expression level of one or more gene products of the subject's molecular signature to an expression level of the one or more gene products of a reference molecular signature, wherein an increase in expression level of the one or more gene products of the subject's molecular signature is indicative of a disease state. In some embodiments, analyzing comprises comparing an expression level of one or more gene products of the subject's molecular signature to an expression level of the one or more gene products of a reference molecular signature, wherein a decrease in expression level of the one or more gene products of the subject's molecular signature is indicative of a disease state. In some cases, the disease state is melanoma stage 0. In some cases, the disease state is melanoma stage 1. In some cases, the disease state is melanoma stage 2. In some cases, the disease state is melanoma stage 3. In some cases, the disease state is melanoma stage 4.

In another aspect, the disclosure provides a method of determining a likelihood of a subject responding to a melanoma treatment. In one embodiment, the method includes determining and analyzing a molecular signature of a subject comprising 1, 2, 3, 4, 5, or 6 of the following genes or gene expression products WFDC3, SLC16A6, DUSP4, PPAP2A, NDUFAF4 and BCL2A1. In some embodiments, the method includes further analyzing 1, 2, 3, 4, 5, 6, 7, 8, 9, 10, 11, 12, 13, or 14 genes or gene expression products selected from DMXL2, SMG1, SLC2A3, VEGFA, BEX4, SH3BP5, RAB27A, FEM1A, CYTH3, IRGQ, EFNA5, NAMPT, PNPT1, and HNRNPH3. In some embodiments, the method includes analyzing 1, 2, 3, 4, 5, 6, 7, 8, 9, 10, 11, 12, 13, 14, 15, 16, 17, 18, 19, or 20 of the following genes or gene expression products WFDC3, SLC16A6, DUSP4, PPAP2A, NDUFAF4, BCL2A1, DMXL2, SMG1, SLC2A3, VEGFA, BEX4, SH3BP5, RAB27A, FEM1A, CYTH3, IRGQ, EFNA5, NAMPT, PNPT1, and HNRNPH3. In some embodiments, analyzing comprises comparing the expression levels of the subject's molecular signature to one or more reference molecular signatures, wherein the one or more reference molecular signatures correspond to samples from patients who have responded to a melanoma treatment, samples from patients who have not responded to a melanoma treatment, or a combination thereof; and wherein if the subject's molecular signature best matches to a reference molecular signature, the subject is determined to respond or not respond to the melanoma treatment. In some embodiments, analyzing comprises comparing an expression level of one or more gene products of the subject's molecular signature to an expression level of the one or more gene products of a reference molecular signature, wherein an increase in expression level of the one or more gene products of the subject's molecular signature is indicative of a subject's likelihood for responding to a melanoma treatment. In some embodiments, analyzing comprises comparing an expression level of one or more gene products of the subject's molecular signature to an expression level of the one or more gene products of a reference molecular signature, wherein a decrease in expression level of the one or more gene products of the subject's molecular signature is indicative of a subject's likelihood for responding to a melanoma treatment.

In another aspect, the disclosure provides a method of monitoring the progression of melanoma. In some embodiments, during the progression of melanoma, one or more treatments are provided to the subject with melanoma. In some instances, the treatment is a cancer therapy such as radiation. In some instances, the treatment is a biopsy of the pigmented skin lesion comprising the melanoma. In one embodiment, the method includes determining and analyzing a molecular signature of a subject comprising 1, 2, 3, 4, 5, or 6 of the following genes or gene expression products WFDC3, SLC16A6, DUSP4, PPAP2A, NDUFAF4 and BCL2A1. In some embodiments, the method includes further analyzing 1, 2, 3, 4, 5, 6, 7, 8, 9, 10, 11, 12, 13, or 14 genes or gene expression products selected from DMXL2, SMG1, SLC2A3, VEGFA, BEX4, SH3BP5, RAB27A, FEM1A, CYTH3, IRGQ, EFNA5, NAMPT, PNPT1, and HNRNPH3. In some embodiments, the method includes analyzing 1, 2, 3, 4, 5, 6, 7, 8, 9, 10, 11, 12, 13, 14, 15, 16, 17, 18, 19, or 20 of the following genes or gene expression products selected from WFDC3, SLC16A6, DUSP4, PPAP2A, NDUFAF4, BCL2A1, DMXL2, SMG1, SLC2A3, VEGFA, BEX4, SH3BP5, RAB27A, FEM1A, CYTH3, IRGQ, EFNA5, NAMPT, PNPT1, and HNRNPH3. In some embodiments, analyzing comprises comparing the expression levels of the subject's molecular signature at a first time point to expression levels of the subject's molecular signature at one or more subsequent time points, wherein if the expression level of one or more genes within the molecular signature is increased at a subsequent time point, the melanoma progressed.

subject's molecular signature best matches to a reference molecular signature, the subject is determined to respond or not respond to the melanoma treatment. In some embodiments, analyzing comprises comparing an expression level of one or more gene products of the subject's molecular signature to an expression level of the one or more gene products of a reference molecular signature, wherein an increase in expression level of the one or more gene products of the subject's molecular signature is indicative of a subject's likelihood for responding to a melanoma treatment. In some embodiments, analyzing comprises comparing an expression level of one or more gene products of the subject's molecular signature to an expression level of the one or more gene products of a reference molecular signature, wherein a decrease in expression level of the one or more gene products of the subject's molecular signature is indicative of a subject's likelihood for responding to a melanoma treatment.

In some embodiments, the molecular signature of a subject is a gene expression profile obtained by analyzing gene expression products of target genes from a subject. In some embodiments, the gene expression profile of a subject is compared to a reference gene expression profile of a reference sample. In some embodiments, the reference sample is a control sample. In some embodiments, the control gene expression profile comprises a relative amount of the target gene expression products in a normal epidermal skin sample not having melanoma. In some embodiments, the relative amount of the gene expression product in a subject is decreased compared to the control by about 2-fold, 3-fold, 4-fold, 5-fold, 10-fold, 20-fold, 30-fold, 40-fold, 50-fold, 60-fold, 70-fold, 80-fold, 90-fold, or 100-fold. In some embodiments, the relative amount of the gene expression product in a subject is increased compared to the control by about 2-fold, 3-fold, 4-fold, 5-fold, 10-fold, 20-fold, 30-fold, 40-fold, 50-fold, 60-fold, 70-fold, 80-fold, 90-fold, or 100-fold. In some embodiments, the reference gene expression profile comprises a relative amount of the gene expression products in a skin sample containing melanoma in situ. In some embodiments, the reference gene expression profile comprises a relative amount of gene expression products in a skin sample containing invasive melanoma. In some embodiments, the reference gene expression profile comprises a relative amount of the gene expression products in a skin sample containing stage 0, 1, 2, 3, or 4 melanoma. In some embodiments, the gene expression products are RNA. In some embodiments, the gene expression products are polypeptides.

In some embodiments, a relative amount of a gene expression product in a sample is decreased compared to a reference sample, and this decrease in expression level is indicative of a disease state of the sample. In some cases, the gene expression product is WFDC3. In some embodiments, the gene expression product is SLC16A6. In some embodiments, the gene expression product is DUSP4. In some embodiments, the gene expression product is PPAP2A. In some embodiments, the gene expression product is NDUFAF4. In some embodiments, the gene expression product is BCL2A1. In some embodiments, the gene expression product is DMXL2. In some embodiments, the gene expression product is SMG1. In some embodiments, the gene expression product is SLC2A3. In some embodiments, the gene expression product is VEGFA. In some embodiments, the gene expression product is BEX4. In some embodiments, the gene expression product is SH3BP5. In some embodiments, the gene expression product is RAB27A. In some embodiments, the gene expression product is FEM1A. In some embodiments, the gene expression product is CYTH3. In some embodiments, the gene expression product is IRGQ. In some embodiments, the gene expression product is EFNA5. In some embodiments, the gene expression product is NAMPT. In some embodiments, the gene expression product is PNPT1. In some embodiments, the gene expression product is HNRNPH3. In some cases, the disease state is melanoma. In some cases, the disease state is melanoma in situ. In some cases, the disease state is invasive melanoma.

In some embodiments, a relative amount of a gene expression product in a sample is increased compared to a reference sample, and this increase in expression level is indicative of a disease state of the sample. In some cases, the gene expression product is WFDC3. In some embodiments, the gene expression product is SLC16A6. In some embodiments, the gene expression product is DUSP4. In some embodiments, the gene expression product is PPAP2A. In some embodiments, the gene expression product is NDUFAF4. In some embodiments, the gene expression product is BCL2A1. In some embodiments, the gene expression product is DMXL2. In some embodiments, the gene expression product is SMG1. In some embodiments, the gene expression product is SLC2A3. In some embodiments, the gene expression product is VEGFA. In some embodiments, the gene expression product is BEX4. In some embodiments, the gene expression product is SH3BP5. In some embodiments, the gene expression product is RAB27A. In some embodiments, the gene expression product is FEM1A. In some embodiments, the gene expression product is CYTH3. In some embodiments, the gene expression product is IRGQ. In some embodiments, the gene expression product is EFNA5. In some embodiments, the gene expression product is NAMPT. In some embodiments, the gene expression product is PNPT1. In some embodiments, the gene expression product is HNRNPH3. In some cases, the disease state is melanoma. In some cases, the disease state is melanoma in situ. In some cases, the disease state is invasive melanoma.

In some embodiments, the molecular signature of a subject is a gene sequence profile obtained by analyzing the sequences of target genes from a subject. In some instances, the gene sequence profile of a subject is compared to a gene sequence profile of a reference sample. In some embodiments, the reference sample is a control sample. In some embodiments, the control gene sequence profile comprises target genes in a normal epidermal skin sample. In some embodiments, the reference sample is a sample containing melanoma in situ. In some embodiments, the reference sample is a sample containing invasive melanoma. In some embodiments, the reference sample is a sample containing melanoma in stage 0, 1, 2, 3, or 4.

In some embodiments, a difference in length or sequence between gene fragments isolated from a sample and those of known sequences are due to an insertion, deletion, or substitution of one or more nucleotides, therefore the determination of gene sequences provides information concerning mutations which have influence on the characterization of a pigmented skin lesion in a subject. In some aspects, these mutations also include a transposition or an inversion and are difficult to detect by other techniques than direct sequencing. Accordingly, in some aspects, the methods of the present disclosure are used to detect genetic mutations in 1, 2, 3, 4, 5, 6, 7, 8, 9, 10, 11, 12, 13, 14, 15, 16, 17, 18, 19 or 20 of the following genes or gene expression products WFDC3, SLC16A6, DUSP4, PPAP2A, NDUFAF4, BCL2A1, DMXL2, SMG1, SLC2A3, VEGFA, BEX4, SH3BP5, RAB27A, FEM1A, CYTH3, IRGQ, EFNA5, NAMPT, PNPT1, and HNRNPH3, for characterization of a pigmented skin lesion in a subject.

In some embodiments, the molecular signature of a subject is a protein sequence profile obtained by analyzing the sequences of proteins expressed by target genes from a subject. In some instances, the protein sequence profile of a subject is compared to a protein sequence profile of a reference sample. In some embodiments, the reference sample is a control sample. In some embodiments, the control protein sequence profile comprises expressed target genes in a normal epidermal skin sample. In some embodiments, the reference sample is a sample containing melanoma in situ. In some embodiments, the reference sample is a sample containing invasive melanoma. In some embodiments, the reference sample is a sample containing melanoma in stage 0, 1, 2, 3, or 4.

### Methods for Determining a Molecular Signature

### Gene Expression Profile

The gene expression profile is determined, in part, by detecting and/or quantifying one or more gene expression products present in cellular material obtained from a subject. In some embodiments, cellular material is obtained from a tissue sample isolated from a skin surface. The tissue sample may be isolated using the tape stripping method provided herein. The tissue sample may be isolated by biopsy. In some instances, cellular material is obtained from blood, urine, tear, sweat, hair, plasma, and/or serum sample from the subject. In some embodiments, the one or more gene expression product is fully or partially isolated and/or purified from other cellular material prior to or during the detection and/or quantification of the gene expression product. In some embodiments, the gene expression product is a RNA molecule. In some embodiments, the gene expression product is a polypeptide.

In some embodiments, a microarray is employed for detection and/or quantification of a gene expression product in a gene expression profile. The manufacture and use of biochips such as those involving microarrays, also known as bioarrays, are known in the art (For reviews of Biochips and microarrays see, e.g., Kallioniemi O. P., “Biochip technologies in cancer research,” Ann Med, March; 33(2):142 7 (2001); and Rudert F., “Genomics and proteomics tools for the clinic,” Curr Opin. Mol. Ther., December; 2(6):633 42 (2000)). Furthermore, a number of biochips for expression analysis are commercially available (See e.g., microarrays available from Sigma-Genosys (The Woodlands, Tex.); Affymetrix (Santa Clara, Calif.), and Full Moon Biosystems (Sunnyvale, Calif.)). In some embodiments, such microarrays are analyzed using blotting techniques similar to those discussed below for conventional techniques of detecting polynucleotides and polypeptides. In some embodiments, detailed protocols for hybridization conditions are available through manufacturers of microarrays. In some embodiments, a microarray provides for the detection and analysis of at least 1, 2, 3, 4, 5, 6, 7, 8, 9, 10, 11, 12, 13, 14, 15, 16, 17, 18, 19, 20, 25, 50, 100, 200, 250, 500, 750, 1000, 2500, 5000, 7500, 10,000, 12,500, 25,000, 50,0000, or 100,000 gene expression products. Information regarding the detection and/or quantification of a gene expression profile is a product and/or article of the microarray methods provided herein.

In some embodiments, for microarray expression analysis, isolated and/or purified RNA is amplified. Isolated and/or purified RNA is an article and/or product of the microarray expression analysis. Amplified RNA is an article and/or product of the microarray expression analysis. In some embodiments, the amplified RNA is then used for hybridization to sequence specific nucleic acid probes on a biochip. Hybridized RNA to sequence specific nucleic probes on a biochip is an article and/or product of the microarray expression analysis. In some embodiments, amplification is performed using a commercially available kit, such as MessageAMp™ RNA kit (Ambion Inc.). In some embodiments, isolated and/or purified RNA is labeled before contacting the biochip such that binding to the target array can be detected using streptavidin. In some embodiments, isolated and/or purified RNA is labeled with a detectable moiety, including, but not limited to, a fluorescent moiety, a dye, or a ligand, such as biotin. Labeled RNA is an article and/or product of the microarray expression analysis. In some embodiments, the nucleic acid probes of the microarray bind specifically to 1, 2, 3, 4, 5, 6, 7, 8, 9, 10, 11, 12, 13, 14, 15, 16, 17, 18, 19, or 20 of the following gene expression products WFDC3, SLC16A6, DUSP4, PPAP2A, NDUFAF4, BCL2A1, DMXL2, SMG1, SLC2A3, VEGFA, BEX4, SH3BP5, RAB27A, FEM1A, CYTH3, IRGQ, EFNA5, NAMPT, PNPT1, and HNRNPH3. In some embodiments, the nucleic acid probes of the microarray bind specifically to 1, 2, 3, 4, 5, or 6 of the following gene expression products from the genes WFDC3, SLC16A6, DUSP4, PPAP2A, NDUFAF4, and BCL2A1.

In some embodiments, hybridization of amplified nucleic acids to probes on a microarray is typically performed under stringent hybridization conditions. Conditions for hybridization reactions are well known in the art and are available from microarray suppliers. For example, in some embodiments, hybridization of a nucleic acid molecule with probes found on a microarray is performed under moderately stringent or highly stringent physiological conditions, as are known in the art. For example, in some embodiments, hybridization on a microarray is performed according to manufacturer's (Affymetrix) instructions. For example, in some embodiments, hybridization is performed for 16 hours at 45° C. in a hybridization buffer, such as 100 mM MES, 1 M [Na⁺], 20 mM EDTA, 0.01% Tween 20. In some embodiments, washes are performed in a low stringency buffer ((6×SSPE, 0.01% Tween 20) at 25° C. followed by a high stringency buffer (100 mM MES, 0.1M [Na⁺], 0.01% Tween 20) at 5° C. In some embodiments, washes are performed using progressively higher stringency conditions: 2×SSC/0.1% SDS at about room temperature (hybridization conditions); 0.2×SSC/0.1% SDS at about room temperature (low stringency conditions); 0.2×SSC/0.1% SDS at about 42° C. (moderate stringency conditions); and 0.1×SSC at about 68° C. (high stringency conditions). In some embodiments, washing is carried out using only one of these conditions, for example, high stringency conditions. In some embodiments, washing is carried out using each of the conditions. In some embodiments, washing is carried out using each of the conditions, for 10 to 15 minutes each, in the order listed above, optionally repeating any or all of the steps listed.

In some embodiments, other microfluidic devices and methods for analyzing gene expression products, including those in which more than one gene expression product can be analyzed simultaneously and those involving high-throughput technologies, are used for the methods provided herein. Information obtained during and after the analysis of gene expression products is an article and/or product of the methods provided herein.

Quantitative measurement of gene expression levels using bioarrays is also known in the art, and typically involves a modified version of a traditional method for measuring expression as described herein. For example, such quantitation can be performed by measuring a phosphor image of a radioactive-labeled probe binding to a spot of a microarray, using a phospohor imager and imaging software. Information regarding the measured gene expression levels is an article and/or product of the methods provided herein.

In some embodiments, the determined gene expression profile of a subject is analyzed by comparing it to a set of reference gene expression profiles. The reference set of gene expression profiles comprise gene expression products recovered from reference subjects with a known skin characteristic (e.g., melanoma, melanoma in situ, invasive melanoma, stage 1 melanoma, stage 2 melanoma, stage 3 melanoma, and/or stage 4 melanoma). A deviation or correlation between the reference profiles and the subject gene expression profile is used to establish a characterization of melanoma. Many statistical techniques are known in the art, which can be used to determine whether a statistically significant difference or correlation in gene expression between a subject's gene expression profile and a reference gene expression profile is observed at a 90% or preferably a 95% confidence level. In some embodiments, a statistical software program or module performed on a computer processor is used to determine whether a statistically significant difference or correlation in gene expression is observed at a given confidence level. Information regarding the statistical significance of a difference or correlation in gene expression level is an article and/or product of the statistical techniques provided herein.

In some embodiments, a RNAse protection assay is used where RNA is the gene expression product to be detected in the method. In this procedure, a labeled antisense RNA probe is hybridized to the complementary polynucleotide in the sample. The remaining unhybridized single-stranded probe is degraded by ribonuclease treatment. The hybridized, double stranded probe is protected from RNAse digestion. After an appropriate time, the products of the digestion reaction are collected and analyzed on a gel (see for example Ausubel et al., CURRENT PROTOCOLS IN MOLECULAR BIOLOGY, section 4.7.1 (1987)). In some embodiments, a software program or module performed on a computer processor is involved in the analysis of the digestion reaction products. Information regarding the analysis of digested reaction products is an article and/or product of the RNAse protection assay. As used herein, “RNA probe” refers to a ribonucleotide capable of hybridizing to RNA in a sample of interest. Those skilled in the art will be able to identify and modify the RNAse protection assay specific to the polynucleotide to be measured, for example, probe specificity, hybridization temperatures, and quantity of nucleic acid can be altered individually or collectively in part or in full. Additionally, a number of commercial kits are available, for example, RiboQuant™ Multi-Probe RNAse Protection Assay System (Pharmingen, Inc., San Diego, Calif.).

In another embodiment, a RNA molecule in a sample is analyzed by a blotting procedure, typically a Northern blot procedure. For blotting procedures RNA molecules are separated on a gel and then probed with a complementary polynucleotide to the sequence of interest. For example, RNA is separated on a gel, transferred to nitrocellulose and probed with complementary DNA to one of the genes disclosed herein. In some embodiments, the complementary probe is labeled radioactively or chemically. In some embodiments, the complementary labeled probe is detected and the Northern blot analyzed using a software program or module performed on a computer processor. Information regarding the analysis of a RNA molecule is an article and/or product of the blotting procedure described herein.

In some embodiments, detection of a RNA molecule includes size fractionation. Methods of size fractionating RNA are well known to those of skill in the art, such as by gel electrophoresis, including polyacrylamide gel electrophoresis (PAGE). For example, in some embodiments, the gel is a denaturing 7 M or 8 M urea-polyacrylamide-formamide gel. In some embodiments, size fractionating the RNA molecule is accomplished by chromatographic methods known to those of skill in the art. In some embodiments, the chromatograph is produced and/or analyzed by a software program or module performed on a computer processor. Information regarding the detection of a RNA molecule, including a chromatograph, is an article and/or product of the size fractionating methods described herein.

In some embodiments, the detection of RNA is performed by using radioactively labeled probes. In some embodiments, any radioactive label is employed which provides an adequate signal. Other labels include ligands, colored dyes, and fluorescent molecules, which, in some embodiments, serve as a specific binding pair member for a labeled ligand, and the like. The labeled preparations are used to probe for a RNA molecule by the Southern or Northern hybridization techniques, for example. RNA obtained from samples are transferred to filters that bind polynucleotides. After exposure to the labeled polynucleotide probe, which will hybridize to RNA nucleotide fragments, the binding of the radioactive probe to RNA fragments is identified by autoradiography (see Genetic Engineering, 1 ed. Robert Williamson, Academic Press (1981), pp. 72 81). In some embodiments, the autoradiograph image is analyzed using a software program or module performed on a computer processor. The analyzed image is an article and/or product of the RNA detection methods provided herein. The particular hybridization technique is not essential to the performance of the method provided. Hybridization techniques are well known or easily ascertained by one of ordinary skill in the art. As improvements are made in hybridization techniques, they can readily be applied in the method of the disclosure.

In some embodiments, probes for use in the methods provided selectively hybridize to a target gene or gene expression product. In some embodiments, the probes are spotted on a bioarray using methods known in the art. As used herein, the term “selective hybridization” or “selectively hybridize,” refers to hybridization under moderately stringent or highly stringent conditions such that a nucleotide sequence preferentially associates with a selected nucleotide sequence over unrelated nucleotide sequences to a large enough extent to be useful in detecting expression of a gene. It will be recognized that some amount of non-specific hybridization is unavoidable, but is acceptable provide that hybridization to a target nucleotide sequence is sufficiently selective such that it can be distinguished over the non-specific cross-hybridization, for example, at least about 2-fold more selective, generally at least about 3-fold more selective, usually at least about 5-fold more selective, and particularly at least about 10-fold more selective, as determined, for example, by an amount of labeled oligonucleotide that binds to target nucleic acid molecule as compared to a nucleic acid molecule other than the target molecule, particularly a substantially similar (i.e., homologous) nucleic acid molecule other than the target nucleic acid molecule.

In some embodiments, conditions that allow for selective hybridization are determined empirically, or estimated based, for example, on the relative GC:AT content of the hybridizing oligonucleotide and the sequence to which it is to hybridize, the length of the hybridizing oligonucleotide, and the number, if any, of mismatches between the oligonucleotide and sequence to which it is to hybridize (see, for example, Sambrook et al., “Molecular Cloning: A laboratory manual (Cold Spring Harbor Laboratory Press 1989)). An example of progressively higher stringency conditions is as follows: 2×SSC/0.1% SDS at about room temperature (hybridization conditions); 0.2×SSC/0.1% SDS at about room temperature (low stringency conditions); 0.2×SSC/0.1% SDS at about 42EC (moderate stringency conditions); and 0.1×SSC at about 68EC (high stringency conditions). In some embodiments, washing is carried out using only one of these conditions, e.g., high stringency conditions, or each of the conditions can be used, e.g., for 10-15 minutes each, in the order listed above, repeating any or all of the steps listed. However, as mentioned above, optimal conditions will vary, depending on the particular hybridization reaction involved, and can be determined empirically.

In another embodiment, provided are methods for obtaining gene expression data from amplified nucleic acids that compensates for variability in amplification reactions. In this method, relative expression of a target gene and a control gene is compared to obtain relevant expression data. In some embodiments, the expression comparison is accomplished by utilizing a software program or module performed on a computer processor. Accordingly, in certain embodiments, a ΔCt value is determined in order to identify gene expression changes. In some embodiments, this value and method is used to identify differential gene expression in any sample containing cellular material, including tissue obtained from the tape stripped methods provided herein. Such method is especially useful, where it is relatively difficult to obtain sufficient RNA from a control sample. In some embodiments, the ΔCt value is determined using a software program or module performed on a computer processor. The ΔCt value and related gene expression data are articles and/or products of the method provided herein.

The Cₜ value is the experimentally determined number of amplification (e.g. PCR) cycles required to achieve a threshold signal level (statistically significant increase in signal level (e.g. fluorescence) over background) for mRNAₓ and a reference or control mRNA (Gibson, Heid et al. 1996; Heid, Stevens et al. 1996). The Ct values are typically determined using a target nucleic acid (e.g. mRNAx) primer and probe set, and a reference or control mRNA primer and probe set. A Δ Cₜ value is calculated by calculating a difference in the number of amplification cycles required to reach a threshold signal level between the target nucleic acid molecule and the reference or control nucleic acid molecule. A difference in the Δ Cₜ value at a target area versus another area of a subject's skin, such as a normal area, or an unaffected area, is indicative of differential gene expression of the target nucleic acid molecule at the target area. A difference in the Δ Cₜ value at a target area versus another area from a reference sample with a melanoma characteristic, is indicative of differential gene expression of the target nucleic acid molecule at the target area. Using this value, differential expression is detected by comparing expression of the target nucleic acid molecule with expression of a control nucleic acid molecule. Using this value, correlated expression is detected by comparing expression of the target nucleic acid molecule with expression of a control nucleic acid molecule. In some embodiments, the comparison is performed by a software program or module performed on a computer. The Δ Cₜ value is useful for characterizing the physiologic state of the skin without reference to a calibration site. Such methods provide the advantage that it is not necessary to obtain a nucleic acid sample from a control site, where it may be difficult to obtain sufficient nucleic acid molecules. In some embodiments, the ΔCt value is determined using a software program or module performed on a computer processor. The ΔCt value and information regarding the characteristic of a target area of skin are articles and/or products of the method provided herein.

Accordingly, provided herein is a method for detecting a difference or correlation between gene expression profiles in a subject and a reference or control sample, including: applying an adhesive tape to a pigmented skin lesion in the subject in a manner sufficient to isolate an epidermal sample adhering to the adhesive tape, wherein the epidermal sample comprises a gene or gene expression product. In certain aspects, the method is used to detect an expression level for 1, 2, 3, 4, 5, 6, 7, 8, 9, 10, 11, 12, 13, 14, 15, 16, 17, 18, 19, or 20 of the following genes WFDC3, SLC16A6, DUSP4, PPAP2A, NDUFAF4, BCL2A1, DMXL2, SMG1, SLC2A3, VEGFA, BEX4, SH3BP5, RAB27A, FEM1A, CYTH3, IRGQ, EFNA5, NAMPT, PNPT1, and HNRNPH3 to assist in a characterization of a pigmented skin lesion. In some embodiments the gene expression product is a nucleic acid, such as RNA, which is then amplified. In some embodiments, RNA from a reference or control sample is isolated and amplified. In some instances, the reference or control RNA is isolated and/or amplified at a different point in time than the RNA obtained from the subject. In some embodiments, information regarding the amplified reference or control RNA is maintained in a reference or control profile for comparison to information obtained from the subject. In some embodiments, a Δ Cₜ value is obtained by calculating a difference in the number of amplification cycles required to reach a threshold signal level between the RNA obtained from the subject and the reference or control RNA, wherein a difference in the Δ Cₜ value is indicative of a characteristic of the pigmented skin lesion in a subject. In some embodiments, a Δ Cₜ value is obtained by calculating a difference in the number of amplification cycles required to reach a threshold signal level between the RNA obtained from the subject and the reference or control RNA, wherein a correlation in the Δ Cₜ value is indicative of a characteristic of the pigmented skin lesion in a subject. In some embodiments, the target and control nucleic acids are identified using a software program or module performed on a computer processor. In some embodiments, Δ Cₜ value is obtained using a software program or module performed on a computer processor. In some embodiments, the Δ Ct values are determined in the same amplification experiment (e.g. using separate reaction wells on the same multi-well reaction plate) using similar reaction conditions to other reactions. In some embodiments, the ΔCt value is determined using a software program or module performed on a computer processor. The ΔCt value and information regarding the characteristic of a pigmented skin lesion are articles and/or products of the method provided herein.

In some embodiments, the method for detecting a gene expression profile is used along with the other embodiments provided herein to characterize a pigmented skin lesion, especially a pigmented skin lesion having melanoma. For example, in some embodiments, the method is used to diagnose a pigmented skin lesion as being melanoma. In some embodiments, the method is used to identify a stage of melanoma, for example stage 0, 1, 2, 3, or 4. In some embodiments, the method is used to identify invasive melanoma. In some embodiments, the method is used to distinguish between melanoma in situ and invasive melanoma. In some embodiments, the method is used to identify a subject as susceptible for a melanoma treatment. In some embodiments, the method is used for monitoring the progression of melanoma. In some embodiments, the method is used for predicting if melanoma will become invasive melanoma. In certain aspects, the method is used to detect the level of RNA expression products for 1, 2, 3, 4, 5, 6, 7, 8, 9, 10, 11, 12, 13, 14, 15, 16, 17, 18, 19, or 20 of the following genes WFDC3, SLC16A6, DUSP4, PPAP2A, NDUFAF4, BCL2A1, DMXL2, SMG1, SLC2A3, VEGFA, BEX4, SH3BP5, RAB27A, FEM1A, CYTH3, IRGQ, EFNA5, NAMPT, PNPT1, and HNRNPH3 in a sample to assist in a characterization of a pigmented skin lesion. In certain aspects, the method is used to detect the level of RNA expression products for 1, 2, 3, 4, 5, or 6 of the following genes WFDC3, SLC16A6, DUSP4, PPAP2A, NDUFAF4, BCL2A1 to assist in a characterization of a pigmented skin lesion. The gene expression profiles are articles and/or products of the methods described herein. Information regarding a melanoma characteristic is an article and/or product of the methods described herein.

In some embodiments, the gene expression profile is determined by identifying and/or quantifying one or more polypeptide products present in cellular material obtained from a subject. In some instances, cellular material is obtained from a tissue sample isolated from a skin surface. The tissue sample may be isolated using the tape stripping method provided herein. In some instances, cellular material is obtained from a blood, urine, tear, sweat, hair, plasma, and/or serum sample from the subject. In some embodiments, a polypeptide is fully or partially isolated and/or purified from other cellular material prior to or during the detection of the polypeptide. In some instances, a polypeptide is isolated and/or purified from other cellular materials by standard protein purification techniques including, but limited to, ammonium sulfate precipitation, ion exchange chromatography, size exclusion chromatography, affinity chromatography, immunoprecipitation, ultracentrifugation, hydrophobicity chromatography, and any combination thereof. The isolated and/or partially purified polypeptides are articles and/or products of the methods described herein. Information regarding the identification and/or quantification of a polypeptide are articles and/or products of the methods described herein.

In some embodiments, 1, 2, 3, 4, 5, 6, 7, 8, 9, 10, 11, 12, 13, 14, 15, 16, 17, 18, 19, or 20 polypeptide products expressed from the following genes WFDC3, SLC16A6, DUSP4, PPAP2A, NDUFAF4, BCL2A1, DMXL2, SMG1, SLC2A3, VEGFA, BEX4, SH3BP5, RAB27A, FEM1A, CYTH3, IRGQ, EFNA5, NAMPT, PNPT1, and HNRNPH3 are detected and/or quantified in a sample to determine a gene expression profile. In some embodiments, 1, 2, 3, 4, 5, or 6 polypeptide products expressed from the following genes WFDC3, SLC16A6, DUSP4, PPAP2A, NDUFAF4, and BCL2A1 are detected and/or quantified in a sample to determine a gene expression profile. In some embodiments, the levels of such polypeptide gene expression products are indicative of melanoma when compared to reference or control polypeptide products in a similar sample. In some instances, the sample is a tissue sampled from the skin of a subject. In this regard, the sample, as described herein, is used as a source to isolate polypeptides. For example, in some embodiments, following skin sampling using the tape stripping methods provided herein, cells isolated from the stratum corneum are lysed by any number of means, and polypeptides are obtained from the cells. In some embodiments, these polypeptides are identified and/or quantified using detection methods known to those of skill in the art, for example by protein microarrays, ELISA, immunohistochemistry, immunophenotyping, fluorescent in situ hybridization (FISH), mass spectrometry, absorbance measurement, and/or any combination thereof. In some embodiments, polypeptide gene expression products are identified and/or quantified using either polyclonal or monoclonal antibodies specific for the protein expression product. Examples include enzyme-linked immunosorbent assay (ELISA), radioimmunoassay (RIA), and fluorescence activated cell sorting (FACS). These and other assays are described, among other places, in Hampton, R. et al. (1990; Serological Methods, a Laboratory Manual, APS Press, St Paul, Minn.) and Maddox, D. E. et al. (1983; J. Exp. Med. 158:1211-1216). In some embodiments, the gene expression profile is obtained and/or analyzed using a software program or module performed on a computer processor. The gene expression profile and information regarding the gene expression profile are articles and/or products of the methods described herein.

### Gene Sequence Profile

The gene sequence profile is determined by identifying the DNA sequence of one or more genes present in cellular material obtained from a subject. In some embodiments, determining the DNA sequence includes determining the sequence of one or nucleic acids, where the sequence of the one or more nucleic acids is indicative of a melanoma characteristic. The DNA sequence includes coding, non-coding DNA, or both coding and non-coding DNA. In some instances, the DNA sequence includes a single-nucleotide polymorphism. In some instances, cellular material is obtained from a tissue sample isolated using the tape stripping method provided herein. In some instances, cellular material is obtained from skin, tissue, blood, urine, tear, sweat, hair, plasma, and/or serum sample from the subject. In some embodiments, DNA is fully or partially isolated and/or purified from other cellular material prior to or during DNA sequencing. In some instances, DNA is isolated and/or purified from other cellular materials by standard DNA purification techniques including, but not limited to, organic extraction (phenol, chloroform, and/or isoamyl alcohol), cesium chloride density gradients, anion-exchange methods, and selective adsorption to silica. Commercial kits available to at least partially isolate and/or purify DNA from cellular material include, but are not limited to, QIAamp (Qiagen), DNeasy (Qiagen), Quick-gDNA™ (Zymo Research), ZR-96 Quick-gDNA (Zymo Research), Xpedition™ (Zymo Research), DNAzol® (Life Technologies), ChargeSwitch® gDNA Mini Tissue Kit (Life Technologies), PureLink® (Life Technologies), GeneCatcher™ (Life Technologies), ChargeSwitch® Forensic DNA Purification Kit (Life Technologies), ReliaPrep™ (Promega), and Wizard® (Promega). In some embodiments, the gene sequence profile is obtained and/or analyzed using a software program or module performed on a computer processor. Isolated cellular material and any product isolated and/or purified from the cellular material are articles and/or products of the methods provided herein. The gene sequence profile and information regarding the gene sequence profile are articles and/or products of the methods described herein.

In some embodiments, the DNA of 1, 2, 3, 4, 5, 6, 7, 8, 9, 10, 11, 12, 13, 14, 15, 16, 17, 18, 19, or 20 of the following genes WFDC3, SLC16A6, DUSP4, PPAP2A, NDUFAF4, BCL2A1, DMXL2, SMG1, SLC2A3, VEGFA, BEX4, SH3BP5, RAB27A, FEM1A, CYTH3, IRGQ, EFNA5, NAMPT, PNPT1, and HNRNPH3 are sequenced in a sample to determine a gene sequence profile. In some embodiments, the DNA of 1, 2, 3, 4, 5, or 6 of the following genes WFDC3, SLC16A6, DUSP4, PPAP2A, NDUFAF4, and BCL2A1 are sequenced in a sample to determine a gene sequence profile. In some embodiments, the DNA sequence is indicative of melanoma when compared to a reference or control sequencing profile in a similar sample. In some instances, the sample is a tissue sampled from the skin of a subject. In this regard, the sample, as described herein, is used as a source to isolate DNA. For example, in some embodiments, following skin stripping, using the methods described herein, cells isolated and/or purified from the stratum corneum are lysed by any number of means, and DNA is obtained from the cells. In some instances, DNA is sequenced by traditional sequencing methods (see, A. M. Maxam and W. Gilbert, Proc. Natl. Acad. Sci. USA 74:560 (1977); Sanger et al., Proc. Natl. Acad. Sci. USA 74:5463 (1977)), next-generation sequencing methods (see, Mardis E R, Annu. Rev. Genomics Hum. Genet. USA 9:387 (2008)), or additional methods known to one of skill in the art. In some embodiments, the gene sequence profile is obtained and/or analyzed using a software program or module performed on a computer processor. The gene sequence profile and information regarding the gene sequence profile are articles and/or products of the methods described herein.

### Protein Sequence Profile

The protein sequence profile is determined by identifying a polypeptide sequence of one or more proteins present in cellular material obtained from a subject. In some embodiments, determining the polypeptide sequence includes determining the sequence of one or more amino acids, where the sequence of the one or more amino acids is indicative of a melanoma characteristic. Determination of the amino acid sequence can include determining the presence or absence of a specific amino acid sequence, for example, but using a detectable probe such as an antibody to detect a specific amino acid sequence. In some instances, the polypeptide includes post-translational modifications including, but not limited to, glycosylation, phosphorylation, ubiquitination, S-nitrosylation, methylation, N-acetylation, lipidation, and/or proteolysis. In some embodiments, a post-translational modification is indicative of a melanoma characteristic. In some instances, cellular material is obtained from a tissue sample isolated from a skin surface. In some embodiments, the tissue sample is isolated using the tape stripping method provided herein. In some instances, cellular material is obtained from tissue, blood, urine, tear, sweat, hair, plasma, and/or serum sample from the subject. In some embodiments, a polypeptide is fully or partially isolated and/or purified from other cellular material prior to or during the detection of the polypeptide. In some instances, a polypeptide is isolated and/or purified from other cellular materials by standard protein purification techniques including ammonium sulfate precipitation, ion exchange chromatography, size exclusion chromatography, affinity chromatography, immunoprecipitation, ultracentrifugation, hydrophobicity chromatography, and any combination thereof. In some embodiments, the protein sequence profile is obtained and/or analyzed using a software program or module performed on a computer processor. The isolated and/or purified polypeptide is an article and/or product of the methods described herein. The protein sequence profile and information regarding the protein sequence profile are articles and/or products of the methods described herein.

In some embodiments, the identity of 1, 2, 3, 4, 5, 6, 7, 8, 9, 10, 11, 12, 13, 14, 15, 16, 17, 18, 19, or 20 polypeptide products expressed from one or more of the following genes WFDC3, SLC16A6, DUSP4, PPAP2A, NDUFAF4, BCL2A1, DMXL2, SMG1, SLC2A3, VEGFA, BEX4, SH3BP5, RAB27A, FEM1A, CYTH3, IRGQ, EFNA5, NAMPT, PNPT1, and HNRNPH3 determines a protein expression profile. In some embodiments, the identity of 1, 2, 3, 4, 5, or 6 polypeptide products expressed from one or more of the following genes WFDC3, SLC16A6, DUSP4, PPAP2A, NDUFAF4, BCL2A1, DMXL2, SMG1, SLC2A3, VEGFA, BEX4, SH3BP5, RAB27A, FEM1A, CYTH3, IRGQ, EFNA5, NAMPT, PNPT1, and HNRNPH3 determines a protein expression profile. In some instances, the identity of a polypeptide includes a sequence of any number of amino acids in the polypeptide. In some instances, the identity of a polypeptide includes a post-translational modification. In some embodiments, the identity of the polypeptide is indicative of melanoma when compared to a reference or control polypeptide profile in a similar sample. In some instances, the sample is a tissue sampled from the skin of a subject. In this regard, the sample, as described herein, is used as a source to isolate polypeptides. For example, in some embodiments, following skin stripping, using the methods described herein, cells isolated and/or purified from the stratum corneum are lysed by any number of means, and polypeptides are obtained from the cells. In some embodiments, these polypeptides are sequenced using methods known to those of skill in the art, for example by protein microarrays, mass spectrometry, Edman degradation, and/or any combination thereof. In some instances, an amino acid sequence is determined to be present or not in a sample by the use of antibodies specific for the amino acid sequence of interest, for example, in an ELISA, lateral flow assay, or Western Blot. In some embodiments, the protein sequence profile is obtained and/or analyzed using a software program or module performed on a computer processor. The isolated and/or purified polypeptide is an article and/or product of the methods described herein. The protein sequence profile and information regarding the protein sequence profile are articles and/or products of the methods described herein.

### Methods for Analyzing a Molecular Signature

Described herein, in some embodiments, is a method for characterizing a pigmented skin lesion in a subject, comprising analyzing a gene or gene expression product from 1, 2, 3, 4, 5, 6, 7, 8, 9, 10, 11, 12, 13, 14, 15, 16, 17, 18, 19, or 20 of the following genes comprising WFDC3, SLC16A6, DUSP4, PPAP2A, NDUFAF4, BCL2A1, DMXL2, SMG1, SLC2A3, VEGFA, BEX4, SH3BP5, RAB27A, FEM1A, CYTH3, IRGQ, EFNA5, NAMPT, PNPT1, and HNRNPH3, in a sample from the subject, thereby producing a molecular signature indicative of a characteristic of the pigmented skin lesion. In some embodiments, the sample is a sample of the pigmented skin lesion from the subject. In some embodiments, 1, 2, 3, 4, 5, or 6 of the following genes or gene expression products WFDC3, SLC16A6, DUSP4, PPAP2A, NDUFAF4, BCL2A1 are analyzed. In some embodiments, the molecular signature is obtained and/or analyzed using a software program or module performed on a computer processor. The molecular signature is an article and/or product of the method provided herein.

In some embodiments, a two-gene molecular signature for the characterization of melanoma is analyzed (e.g., two genes or gene expression products are analyzed to calculate a molecular signature). In some embodiments, the molecular signature comprises WFDC3 and SLC16A6. In some embodiments, the molecular signature comprises WFDC3 and DUSP4. In some embodiments, the molecular signature comprises WFDC3 and PPAP2A. In some embodiments, the molecular signature comprises WFDC3 and NDUFAF4. In some embodiments, the molecular signature comprises WFDC3 and BCL2A1. In some embodiments, the molecular signature comprises SLC16A6 and DUSP4. In some embodiments, the molecular signature comprises SLC16A6 and PPAP2A. In some embodiments, the molecular signature comprises SLC16A6 and NDUFAF4. In some embodiments, the molecular signature comprises SLC16A6 and BCL2A1. In some embodiments, the molecular signature comprises DUSP4 and PPAP2A. In some embodiments, the molecular signature comprises DUSP4 and NDUFAF4. In some embodiments, the molecular signature comprises DUSP4 and BCL2A1. In some embodiments, the molecular signature comprises PPAP2A and NDUFAF4. In some embodiments, the molecular signature comprises PPAP2A and BCL2A1. In some embodiments, the molecular signature comprises NDUFAF4 and BCL2A1. In some embodiments, the two-gene molecular signature is obtained and/or analyzed using a software program or module performed on a computer processor. The two-gene molecular signature is an article and/or product of the analysis provided herein.

In some embodiments, the two-gene molecular signature has a greater than about 75% or 90% confidence interval for distinguishing non-melanoma from melanoma. In some embodiments, the two-gene molecular signature has a greater than about 95% confidence interval for distinguishing non-melanoma from melanoma. In some embodiments, the two-gene molecular signature has a greater than about 97% confidence interval for distinguishing non-melanoma from melanoma. In some embodiments, the two-gene molecular signature has a greater than about 99% confidence interval for distinguishing non-melanoma from melanoma. The confidence interval is an article and/or product of the two-gene molecular signature described herein.

In some embodiments, the two-gene molecular signature has a greater than about 75% or 90% confidence interval for distinguishing melanoma in situ from invasive melanoma. In some embodiments, the two-gene molecular signature has a greater than about 95% confidence interval for distinguishing melanoma in situ from invasive melanoma. In some embodiments, the two-gene molecular signature has a greater than about 97% confidence interval for distinguishing melanoma in situ from invasive melanoma. In some embodiments, the two-gene molecular signature has a greater than about 99% confidence interval for distinguishing melanoma in situ from invasive melanoma. The confidence interval is an article and/or product of the two-gene molecular signature described herein.

In some embodiments, the two-gene molecular signature has a greater than about 75% or 90% confidence interval for identifying a stage of melanoma. In some embodiments, the two-gene molecular signature has a greater than about 95% confidence interval for identifying a stage of melanoma. In some embodiments, the two-gene molecular signature has a greater than about 97% confidence interval for identifying a stage of melanoma. In some embodiments, the two-gene molecular signature has a greater than about 99% confidence interval for identifying a stage of melanoma. The confidence interval is an article and/or product of the two-gene molecular signature described herein.

In some embodiments, the two-gene molecular signature has a greater than about 75% or 90% confidence interval for identifying melanoma in situ in a sample. In some embodiments, the two-gene molecular signature has a greater than about 95% confidence interval for identifying melanoma in situ in a sample. In some embodiments, the two-gene molecular signature has a greater than about 97% confidence interval for identifying melanoma in situ in a sample. In some embodiments, the two-gene molecular signature has a greater than about 99% confidence interval for identifying melanoma in situ in a sample. The confidence interval is an article and/or product of the two-gene molecular signature described herein.

In some embodiments, the two-gene molecular signature has a greater than about 75% or 90% confidence interval for identifying invasive melanoma in a sample. In some embodiments, the two-gene molecular signature has a greater than about 95% confidence interval for identifying invasive melanoma in a sample. In some embodiments, the two-gene molecular signature has a greater than about 97% confidence interval for identifying invasive melanoma in a sample. In some embodiments, the two-gene molecular signature has a greater than about 99% confidence interval for identifying invasive melanoma in a sample. The confidence interval is an article and/or product of the two-gene molecular signature described herein.

In some embodiments, a three-gene molecular signature for the characterization of melanoma is analyzed (e.g., three genes or gene expression products are analyzed to calculate the molecular signature). In some embodiments, the three-gene molecular signature has a greater than about 75%, 90%, 95%, 97%, or 99% confidence interval for distinguishing non-melanoma from melanoma. In some embodiments, the three-gene molecular signature has a greater than about 75%, 90%, 95%, 97%, or 99% confidence interval for distinguishing melanoma in situ from invasive melanoma. In some embodiments, the three-gene molecular signature has a greater than about 75%, 90%, 95%, 97%, or 99% confidence interval for identifying a stage of melanoma (e.g., 0, 1, 2, 3, or 4). In some embodiments, the three-gene molecular signature has a greater than about 75%, 90%, 95%, 97%, or 99% confidence interval for identifying melanoma in situ in a sample. In some embodiments, the three-gene molecular signature has a greater than about 75%, 90%, 95%, 97%, or 99% confidence interval for identifying invasive melanoma in a sample. In some embodiments, the three-gene molecular signature comprises 1, 2, or 3 of the following genes WFDC3, SLC16A6, DUSP4, PPAP2A, NDUFAF4, and BCL2A1. In some embodiments, the three-gene molecular signature is obtained and/or analyzed using a software program or module performed on a computer processor. The three-gene molecular signature is an article and/or product of the analysis provided herein. The confidence interval is an article and/or product of the three-gene molecular signature described herein.

In some embodiments, a four-gene molecular signature for the characterization of melanoma is analyzed (e.g., four gene or gene expression products are analyzed to calculate the molecular signature). In some embodiments, the four-gene molecular signature has a greater than about 75%, 90%, 95%, 97%, or 99% confidence interval for distinguishing non-melanoma from melanoma. In some embodiments, the four-gene molecular signature has a greater than about 75%, 90%, 95%, 97%, or 99% confidence interval for distinguishing melanoma in situ from invasive melanoma. In some embodiments, the four-gene molecular signature has a greater than about 75%, 90%, 95%, 97%, or 99% confidence interval for identifying a stage of melanoma (e.g., 0, 1, 2, 3, or 4). In some embodiments, the four-gene molecular signature has a greater than about 75%, 90%, 95%, 97%, or 99% confidence interval for identifying melanoma in situ in a sample. In some embodiments, the four-gene molecular signature has a greater than about 75%, 90%, 95%, 97%, or 99% confidence interval for identifying invasive melanoma in a sample. In some embodiments, the four-gene molecular signature comprises 1, 2, 3, or 4 of the following genes WFDC3, SLC16A6, DUSP4, PPAP2A, NDUFAF4, and BCL2A1. In some embodiments, the four-gene molecular signature is obtained and/or analyzed using a software program or module performed on a computer processor. The four-gene molecular signature is an article and/or product of the analysis provided herein. The confidence interval is an article and/or product of the four-gene molecular signature described herein.

In some embodiments, a five-gene molecular signature for the characterization of melanoma is analyzed (e.g., five genes or gene expression products are analyzed to calculate the molecular signature). In some embodiments, the five-gene molecular signature has a greater than about 75%, 90%, 95%, 97%, or 99% confidence interval for distinguishing non-melanoma from melanoma. In some embodiments, the five-gene molecular signature has a greater than about 75%, 90%, 95%, 97%, or 99% confidence interval for distinguishing melanoma in situ from invasive melanoma. In some embodiments, the five-gene molecular signature has a greater than about 75%, 90%, 95%, 97%, or 99% confidence interval for identifying a stage of melanoma (e.g., 0, 1, 2, 3, or 4). In some embodiments, the five-gene molecular signature has a greater than about 75%, 90%, 95%, 97%, or 99% confidence interval for identifying melanoma in situ in a sample. In some embodiments, the five-gene molecular signature has a greater than about 75%, 90%, 95%, 97%, or 99% confidence interval for identifying invasive melanoma in a sample. In some embodiments, the five-gene molecular signature comprises 1, 2, 3, 4, or 5 of the following genes WFDC3, SLC16A6, DUSP4, PPAP2A, NDUFAF4, and BCL2A1. In some embodiments, the five-gene molecular signature is obtained and/or analyzed using a software program or module performed on a computer processor. The five-gene molecular signature is an article and/or product of the analysis provided herein. The confidence interval is an article and/or product of the five-gene molecular signature described herein.

In some embodiments, a six-gene molecular signature for the characterization of melanoma is analyzed (e.g., six genes or gene expression products are analyzed to calculate the molecular signature). In some embodiments, the six-gene molecular signature has a greater than about 75%, 90%, 95%, 97%, or 99% confidence interval for distinguishing non-melanoma from melanoma. In some embodiments, the six-gene molecular signature has a greater than about 75%, 90%, 95%, 97%, or 99% confidence interval for distinguishing melanoma in situ from invasive melanoma. In some embodiments, the six-gene molecular signature has a greater than about 75%, 90%, 95%, 97%, or 99% confidence interval for identifying a stage of melanoma (e.g., 0, 1, 2, 3, or 4). In some embodiments, the six-gene molecular signature has a greater than about 75%, 90%, 95%, 97%, or 99% confidence interval for identifying melanoma in situ in a sample. In some embodiments, the six-gene molecular signature has a greater than about 75%, 90%, 95%, 97%, or 99% confidence interval for identifying invasive melanoma in a sample. In some embodiments, the six-gene molecular signature comprises WFDC3, SLC16A6, DUSP4, PPAP2A, NDUFAF4, and BCL2A1. In some embodiments, the six-gene molecular signature is obtained and/or analyzed using a software program or module performed on a computer processor. The six-gene molecular signature is an article and/or product of the analysis provided herein. The confidence interval is an article and/or product of the six-gene molecular signature described herein.

Described herein, in some embodiments, is a method for identifying a skin lesion in a subject as comprising melanoma or not comprising melanoma, comprising analyzing one or more nucleic acid molecules expression from one or more genes in a melanoma diagnostic signature comprising ACTB, ACTN4, B2M, LINC00518, CNN2, CMIP, EDNRB, GPM6B, KIT, MCOLN3, NAMPT, PPIA, SDCBP, TTC3, TMEM80, TRIB2, TYR, ANKRD11, CASC3, CCL18, CMPK2, CTBP2, DCT, EZR, FOS, FTL, HLA-A, HLA-C, HOXA9, IF16, IRF9, ISG15, KRT17, KTN1, MAFB, MAFK, MAL, MALAT1, MAP1B, MARCKS, MLANA, RNA00188, OTUB1, OVOS2, PKD1P1, PMEL, PRAME, PTPN14, SAT1, SDHA, SIRPA, SOX10, TFRC, TYRP1, UBE2B, VIM, or combination thereof, in a sample of the skin lesion, thereby producing a characterization of the skin lesion. In some embodiments, two or more genes selected from ACTB, ACTN4, B2M, LINC00518, CNN2, CMIP, EDNRB, GPM6B, KIT, MCOLN3, NAMPT, PPIA, SDCBP, TTC3, TMEM80, TRIB2, TYR, ANKRD11, CASC3, CCL18, CMPK2, CTBP2, DCT, EZR, FOS, FTL, HLA-A, HLA-C, HOXA9, IF16, IRF9, ISG15, KRT17, KTN1, MAFB, MAFK, MAL, MALAT1, MAP1B, MARCKS, MLANA, RNA00188, OTUB1, OVOS2, PKD1P1, PMEL, PRAME, PTPN14, SAT1, SDHA, SIRPA, SOX10, TFRC, TYRP1, UBE2B, and VIM are analyzed. In some embodiments, LINC00518 is analyzed. In some embodiments, the genes are selected from LINC00518, TRIB2, KIT, SDCBP, TYR, NAMPT, ACTN4, EDNRB, GPM6B, CNN2, MCOLN3, PRAME, TMEM80, TTC3, and CMIP, and combinations thereof. In some embodiments, the genes are selected from LINC00518, CMIP, ACTN4, TMEM80, PRAME, NAMPT and combinations thereof. In some embodiments, the one or more genes are selected from LINC00518 and CMIP, and combinations thereof. In some embodiments, the combination of LINC00518 and CMIP is analyzed. In some embodiments, the one or more genes are selected from LINC00518, PRAME, and CMIP, and combinations thereof. In some embodiments, the combination of LINC00518, PRAME, and CMIP is analyzed. In some embodiments, the one or more genes are selected from LINC00518 and TMEM80, and combinations thereof. In some embodiments, the combination of LINC00518 and TMEM80 is analyzed. In some embodiments, the one of more genes are selected from LINC00518 and ACTN4, and combinations thereof. In some embodiments, the combination of LINC00518 and ACTN4 is analyzed. In some embodiments, the combination of LINC00518 and NAMPT are analyzed. In some embodiments, the two or more genes comprise LINC00518 and TTC3, and combinations thereof. In some embodiments, the combination of LINC00518 and TTC3 are analyzed. In some embodiments, the two or more genes comprise LINC00518 and HLA-C, and combinations thereof. In some embodiments, the combination of LINC00518 and HLA-C are analyzed. In some embodiments, the two or more genes comprise LINC00518 and PRAME, and combinations thereof. In some embodiments, the combination of LINC00518 and PRAME are analyzed. In some embodiments, the two or more genes comprise ACTN4, LINC00518 and PRAME, and combinations thereof. In some embodiments, the combination of ACTN4, LINC00518 and PRAME are analyzed. In some embodiments, the two or more genes comprise LINC00518 and TRIB2, and combinations thereof. In some embodiments, the two or more genes comprise LINC00518 and TRIB2, and combinations thereof. In some embodiments, the two or more genes comprise LINC00518 and GPM6B, and combinations thereof. In some embodiments, the combination of LINC00518 and GPM6B is analyzed. In some embodiments, the two or more genes comprise TRIB2 and NAMPT, and combinations thereof. In some embodiments, the combination of TRIB2 and NAMPT is analyzed. In some embodiments, the two or more genes comprise TRIB2 and KIT, and combinations thereof. In some embodiments, the combination of TRIB2 and KIT is analyzed. In some embodiments, the combination of TRIB2 and ACTN4 is analyzed. In some embodiments, the combination of TRIB2 and ACTN4 is analyzed.

In some embodiments, the one or more genes or gene expression products analyzed according to the methods described herein have an area under the curve (AUC) of a receiver operating characteristic (ROC) curve (AUC ROC) of greater than about 0.5. In some embodiments, the one or more genes or gene expression products analyzed according to the methods described herein have an AUC ROC of greater than about 0.5, greater than about 0.6, greater than about 0.7, greater than about 0.8, or greater than about 0.9. In some embodiments, the one or more genes or gene expression products analyzed have an AUC ROC of greater than about 0.95. In some embodiments, the ROC curve and AUC ROC are obtained and/or analyzed using a software program or module performed on a computer processor. In some embodiments, the one or more genes or gene expression products are obtained and/or analyzed using a software program or module performed on a computer processor. The AUC ROC is an article and/or product of the methods described herein.

In some embodiments, a molecular signature analyzed according to the methods described herein has an area under the curve (AUC) of a receiver operating characteristic (ROC) curve (AUC ROC) of greater than about 0.5. In some embodiments, a molecular signature analyzed according to the methods described herein has an AUC ROC of greater than about 0.5, greater than about 0.6, greater than about 0.7, greater than about 0.8, or greater than about 0.9. In some embodiments, a molecular signature analyzed according to the methods described herein has an AUC ROC of greater than about 0.95. In some embodiments, the molecular signature is obtained and/or analyzed using a software program or module performed on a computer processor. In some embodiments, the ROC curve and AUC ROC are obtained and/or analyzed using a software program or module performed on a computer processor. The AUC ROC is an article and/or product of the methods described herein.

In some embodiments, a two-gene, three-gene, four-gene, five-gene, or six-gene molecular signature for the characterization of melanoma analyzed according to the methods described herein has an area under the curve (AUC) of a receiver operating characteristic (ROC) curve (AUC ROC) of greater than about 0.5. In some embodiments, a two-gene, three-gene, four-gene, five-gene, or six-gene molecular signature for the characterization of melanoma analyzed according to the methods described herein has an area under the curve (AUC) of a receiver operating characteristic (ROC) curve (AUC ROC) of greater than about 0.5, greater than about 0.6, greater than about 0.7, greater than about 0.8, or greater than about 0.9. In some embodiments, a two-gene, three-gene, four-gene, five-gene, or six-gene molecular signature for the diagnosis of melanoma analyzed according to the methods described herein has an AUC ROC of greater than about 0.95. In some embodiments, the two-gene, three-gene, four-gene, five-gene, or six-gene molecular signature comprises 2, 3, 4, 5, or 6 of the following genes WFDC3, SLC16A6, DUSP4, PPAP2A, NDUFAF4 and BCL2A1. In some embodiments, the molecular signature is obtained and/or analyzed using a software program or module performed on a computer processor. In some embodiments, the ROC curve and AUC ROC are obtained and/or analyzed using a software program or module performed on a computer processor. The AUC ROC is an article and/or product of the methods described herein.

In some embodiments, the one or more genes or gene expression products analyzed according to the methods described herein have a sensitivity of greater than about 50%. In some embodiments, the one or more genes or gene expression products analyzed according to the methods described herein have a sensitivity greater than about 60%, greater than about 65%, greater than about 70%, greater than about 75%, greater than about 80%, greater than about 85%, greater than about 90%, greater than about 93%, greater than about 95%, greater than about 96%, greater than about 97%, greater than about 98%, or greater than about 99%. In some embodiments, the one or more genes or gene expression products analyzed according to the methods described herein have a sensitivity of greater than about 95%. In some embodiments, the one or more genes or gene expression products are analyzed using a software program or module performed on a computer processor. In some embodiments, the sensitivity is obtained and/or analyzed using a software program or module performed on a computer processor. The sensitivity is an article and/or product of the methods described herein.

In some embodiments, a molecular signature analyzed according to the methods described herein has a sensitivity of greater than about 50%. In some embodiments, a molecular signature analyzed according to the methods described herein has a sensitivity of greater than about 60%, greater than about 70%, greater than about 80%, greater than about 90%, greater than about 93%, greater than about 95%, greater than about 96%, greater than about 97%, greater than about 98%, or greater than about 99%. In some embodiments, a molecular signature analyzed according to the methods described herein has a sensitivity of greater than about 95%. In some embodiments, the molecular signature is analyzed using a software program or module performed on a computer processor. In some embodiments, the sensitivity is obtained and/or analyzed using a software program or module performed on a computer processor. The sensitivity is an article and/or product of the methods described herein.

In some embodiments, a two-gene, three-gene, four-gene, five-gene, or six-gene molecular signature analyzed according to the methods described herein has a sensitivity of greater than about 50%. In some embodiments, a two-gene, a two-gene, three-gene, four-gene, five-gene, or six-gene molecular signature analyzed according to the methods described herein has a sensitivity of greater than about 60%, greater than about 65%, greater than about 70%, greater than about 75%, greater than about 80%, greater than about 85%, greater than about 90%, greater than about 93%, greater than about 95%, greater than about 96%, greater than about 97%, greater than about 98%, or greater than about 99%. In some embodiments, a two-gene, three-gene, four-gene, five-gene, or six-gene molecular signature analyzed according to the methods described herein has a sensitivity of greater than about 95%. In some embodiments, the two-gene, a two-gene, three-gene, four-gene, five-gene, or six-gene molecular signature comprises 2, 3, 4, 5, or 6 of the following genes WFDC3, SLC16A6, DUSP4, PPAP2A, NDUFAF4 and BCL2A1. In some embodiments, the two-gene, three-gene, four-gene, five-gene, or six-gene molecular signature is analyzed using a software program or module performed on a computer processor. In some embodiments, the sensitivity is obtained and/or analyzed using a software program or module performed on a computer processor. The sensitivity is an article and/or product of the methods described herein. In some embodiments, a molecular signature is a melanoma diagnostic signature comprising ACTB, ACTN4, B2M, LINC00518, CNN2, CMIP, EDNRB, GPM6B, KIT, MCOLN3, NAMPT, PPIA, SDCBP, TTC3, TMEM80, TRIB2, TYR, ANKRD11, CASC3, CCL18, CMPK2, CTBP2, DCT, EZR, FOS, FTL, HLA-A, HLA-C, HOXA9, IF16, IRF9, ISG15, KRT17, KTN1, MAFB, MAFK, MAL, MALAT1, MAP1B, MARCKS, MLANA, RNA00188, OTUB1, OVOS2, PKD1P1, PMEL, PRAME, PTPN14, SAT1, SDHA, SIRPA, SOX10, TFRC, TYRP1, UBE2B, VIM, or a combination thereof. In some cases, the melanoma diagnostic signature comprises LINC00518, CMIP, PRAME, or any combination thereof.

In some embodiments, the one or more genes or gene expression products analyzed according to the methods described herein have a specificity of greater than about 50%. In some embodiments, the one or more genes or gene expression products analyzed according to the methods described herein have a specificity of greater than about 60%, greater than about 65%, greater than about 70%, greater than about 75%, greater than about 80%, greater than about 85%, greater than about 90%, greater than about 93%, greater than about 95%, greater than about 96%, greater than about 97%, greater than about 98%, or greater than about 99%. In some embodiments, the one or more genes or gene expression products analyzed according to the methods described herein have a specificity of greater than 95%. In some embodiments, the one or more genes or gene expression products are analyzed using a software program or module performed on a computer processor. In some embodiments, the specificity is obtained and/or analyzed using a software program or module performed on a computer processor. The specificity is an article and/or product of the methods described herein.

In some embodiments, a molecular signature analyzed according to the methods described herein has a specificity of greater than about 50%. In some embodiments, a molecular signature analyzed according to the methods described herein has a specificity of greater than about 60%, greater than about 70%, greater than about 80%, greater than about 90%, greater than about 93%, greater than about 95%, greater than about 96%, greater than about 97%, greater than about 98%, or greater than about 99%. In some embodiments, a molecular signature analyzed according to the methods described herein has a specificity of greater than about 95%. In some embodiments, the molecular signature is analyzed using a software program or module performed on a computer processor. In some embodiments, the specificity is obtained and/or analyzed using a software program or module performed on a computer processor. The specificity is an article and/or product of the methods described herein.

In some embodiments, a two-gene, three-gene, four-gene, five-gene, or six-gene molecular signature analyzed according to the methods described herein has a specificity of greater than about 50%. In some embodiments, a two-gene, three-gene, four-gene, five-gene, or six-gene molecular signature analyzed according to the methods described herein has a specificity of greater than about 60%, greater than about 65%, greater than about 70%, greater than about 75%, greater than about 80%, greater than about 85%, greater than about 90%, greater than about 93%, greater than about 95%, greater than about 96%, greater than about 97%, greater than about 98%, or greater than about 99%. In some embodiments, a two-gene, three-gene, four-gene, five-gene, or six-gene molecular signature analyzed according to the methods described herein has a specificity of greater than about 95%. In some embodiments, the two-gene, a two-gene, three-gene, four-gene, five-gene, or six-gene molecular signature comprises 2, 3, 4, 5, or 6 of the following genes WFDC3, SLC16A6, DUSP4, PPAP2A, NDUFAF4 and BCL2A1. In some embodiments, the two-gene, three-gene, four-gene, five-gene, or six-gene molecular signature is analyzed using a software program or module performed on a computer processor. In some embodiments, the specificity is obtained and/or analyzed using a software program or module performed on a computer processor. The specificity is an article and/or product of the methods described herein. In some embodiments, a molecular signature is a melanoma diagnostic signature comprising ACTB, ACTN4, B2M, LINC00518, CNN2, CMIP, EDNRB, GPM6B, KIT, MCOLN3, NAMPT, PPIA, SDCBP, TTC3, TMEM80, TRIB2, TYR, ANKRD11, CASC3, CCL18, CMPK2, CTBP2, DCT, EZR, FOS, FTL, HLA-A, HLA-C, HOXA9, IF16, IRF9, ISG15, KRT17, KTN1, MAFB, MAFK, MAL, MALAT1, MAP1B, MARCKS, MLANA, RNA00188, OTUB1, OVOS2, PKD1P1, PMEL, PRAME, PTPN14, SAT1, SDHA, SIRPA, SOX10, TFRC, TYRP1, UBE2B, VIM, or a combination thereof. In some cases, the melanoma diagnostic signature comprises LINC00518, CMIP, PRAME, or any combination thereof.

In some embodiments, a molecular signature analyzed according to the methods described herein has a sensitivity of greater than about 95% and a specificity of greater than about 70%. In some embodiments, a molecular signature analyzed according to the methods described herein has a sensitivity of greater than about 95% and a specificity of greater than about 80%. In some embodiments, a gene molecular signature analyzed according to the methods described herein has a sensitivity of greater than about 99% and a specificity of greater than about 80%. In some embodiments, the molecular signature is analyzed using a software program or module performed on a computer processor. In some embodiments, the sensitivity and specificity are obtained and/or analyzed using a software program or module performed on a computer processor. The sensitivity and specificity are articles and/or products of the methods described herein.

In some embodiments, a two-gene, three-gene, four-gene, five-gene, or six-gene molecular signature analyzed according to the methods described herein has a sensitivity of greater than about 95% and a specificity of greater than about 70%. In some embodiments, a two-gene, three-gene, four-gene, five-gene, or six-gene molecular signature analyzed according to the methods described herein has a sensitivity of greater than about 95% and a specificity of greater than about 80%. In some embodiments, a two-gene, three-gene, four-gene, five-gene, or six-gene molecular signature analyzed according to the methods described herein has a sensitivity of greater than about 99% and a specificity of greater than about 80%. In some embodiments, the two-gene, a two-gene, three-gene, four-gene, five-gene, or six-gene molecular signature comprises 2, 3, 4, 5, or 6 of the following genes WFDC3, SLC16A6, DUSP4, PPAP2A, NDUFAF4 and BCL2A1. In some embodiments, the two-gene, three-gene, four-gene, five-gene, or six-gene molecular signature is analyzed using a software program or module performed on a computer processor. In some embodiments, the sensitivity and specificity are obtained and/or analyzed using a software program or module performed on a computer processor. The sensitivity and specificity are articles and/or products of the methods described herein. In some embodiments, a molecular signature is a melanoma diagnostic signature comprising ACTB, ACTN4, B2M, LINC00518, CNN2, CMIP, EDNRB, GPM6B, KIT, MCOLN3, NAMPT, PPIA, SDCBP, TTC3, TMEM80, TRIB2, TYR, ANKRD11, CASC3, CCL18, CMPK2, CTBP2, DCT, EZR, FOS, FTL, HLA-A, HLA-C, HOXA9, IF16, IRF9, ISG15, KRT17, KTN1, MAFB, MAFK, MAL, MALAT1, MAP1B, MARCKS, MLANA, RNA00188, OTUB1, OVOS2, PKD1P1, PMEL, PRAME, PTPN14, SAT1, SDHA, SIRPA, SOX10, TFRC, TYRP1, UBE2B, VIM, or a combination thereof. In some cases, the melanoma diagnostic signature comprises LINC00518, CMIP, PRAME, or any combination thereof.

In some embodiments, a limited amount of total nucleic acid is required for performing the methods described herein to determine a gene expression profile or a gene sequencing profile. In some embodiments, the total nucleic acid required for performing the gene expression profile or the gene sequencing profile methods described herein is less than about 1000 ng. In some embodiments, the total nucleic acid required for performing the methods is less than about 500 ng. In some embodiments, the total nucleic acid required for performing the methods is less than about 100 ng. In some embodiments, the total nucleic acid required for performing the methods is less than about 1 ng. In some embodiments, the total nucleic acid required for performing the methods is less than about 500 pg. In some embodiments, the total nucleic acid required for performing the methods is less than about 250 pg. In some embodiments, the nucleic acid is RNA. In some embodiments, the nucleic acid is DNA. In some embodiments, the nucleic acid is cellular material obtained from a tape stripping method provided herein. In some embodiments, the nucleic acid is cellular material obtained from a biopsy. In some embodiments, the cellular material is obtained from tissue, blood, serum, plasma, hair sweat, tears or urine.

In some embodiments, a gene expression profile analyzed according to the methods described herein has a sensitivity of greater than about 95% and a specificity of greater than about 70% when analyzed with about 250-1000 pg input RNA. In some embodiments, a gene expression profile analyzed according to the methods described herein has a sensitivity of greater than 95% and a specificity of greater than 80% when analyzed with about 250-1000 pg input RNA. In some embodiments, a gene expression profile analyzed according to the methods described herein has a sensitivity of greater than 99% and a specificity of greater than about 80% when analyzed with about 250-1000 pg input RNA. In some embodiments, the gene expression profile comprises 2, 3, 4, 5, or 6 of the following genes WFDC3, SLC16A6, DUSP4, PPAP2A, NDUFAF4 and BCL2A1. In some embodiments, the gene expression profile is analyzed using a software program or module performed on a computer processor. In some embodiments, the sensitivity and specificity are obtained and/or analyzed using a software program or module performed on a computer processor. Information regarding the sensitivity and specificity are articles and/or products of the methods described herein. In some embodiments, a molecular signature is a melanoma diagnostic signature comprising ACTB, ACTN4, B2M, LINC00518, CNN2, CMIP, EDNRB, GPM6B, KIT, MCOLN3, NAMPT, PPIA, SDCBP, TTC3, TMEM80, TRIB2, TYR, ANKRD11, CASC3, CCL18, CMPK2, CTBP2, DCT, EZR, FOS, FTL, HLA-A, HLA-C, HOXA9, IF16, IRF9, ISG15, KRT17, KTN1, MAFB, MAFK, MAL, MALAT1, MAP1B, MARCKS, MLANA, RNA00188, OTUB1, OVOS2, PKD1P1, PMEL, PRAME, PTPN14, SAT1, SDHA, SIRPA, SOX10, TFRC, TYRP1, UBE2B, VIM, or a combination thereof. In some cases, the melanoma diagnostic signature comprises LINC00518, CMIP, PRAME, or any combination thereof.

In some embodiments, a gene sequence profile analyzed according to the methods described herein has a sensitivity of greater than about 95% and a specificity of greater than about 70% when analyzed with about 250-1000 pg input DNA. In some embodiments, a gene sequence profile analyzed according to the methods described herein has a sensitivity of greater than 95% and a specificity of greater than 80% when analyzed with about 250-1000 pg input DNA. In some embodiments, a gene sequence profile analyzed according to the methods described herein has a sensitivity of greater than 99% and a specificity of greater than about 80% when analyzed with about 250-1000 pg input DNA. In some embodiments, the gene sequence profile comprises 2, 3, 4, 5, or 6 of the following genes WFDC3, SLC16A6, DUSP4, PPAP2A, NDUFAF4 and BCL2A1. In some embodiments, the gene sequence profile is analyzed using a software program or module performed on a computer processor. In some embodiments, the sensitivity and specificity are obtained and/or analyzed using a software program or module performed on a computer processor. Information regarding the sensitivity and specificity are articles and/or products of the methods described herein. In some embodiments, a molecular signature is a melanoma diagnostic signature comprising ACTB, ACTN4, B2M, LINC00518, CNN2, CMIP, EDNRB, GPM6B, KIT, MCOLN3, NAMPT, PPIA, SDCBP, TTC3, TMEM80, TRIB2, TYR, ANKRD11, CASC3, CCL18, CMPK2, CTBP2, DCT, EZR, FOS, FTL, HLA-A, HLA-C, HOXA9, IF16, IRF9, ISG15, KRT17, KTN1, MAFB, MAFK, MAL, MALAT1, MAP1B, MARCKS, MLANA, RNA00188, OTUB1, OVOS2, PKD1P1, PMEL, PRAME, PTPN14, SAT1, SDHA, SIRPA, SOX10, TFRC, TYRP1, UBE2B, VIM, or a combination thereof. In some cases, the melanoma diagnostic signature comprises LINC00518, CMIP, PRAME, or any combination thereof.

### Identification of Melanoma

Further described herein, in various embodiments, are methods for the identification of melanoma in a subject, wherein if the subject is identified to comprise melanoma, the melanoma is characterized using one or more of the previous methods provided herein. For example, melanoma is identified in the subject, followed by performing a method for characterizing the melanoma as melanoma in situ or invasive melanoma. In some embodiments, a method for characterizing the melanoma comprises analyzing genes or gene expression products expressed from 1, 2, 3, 4, 5, 6, 7, 8, 9, 10, 11, 12, 13, 14, 15, 16, 17, 18, 19, or 20 of the following genes WFDC3, SLC16A6, DUSP4, PPAP2A, NDUFAF4, BCL2A1, DMXL2, SMG1, SLC2A3, VEGFA, BEX4, SH3BP5, RAB27A, FEM1A, CYTH3, IRGQ, EFNA5, NAMPT, PNPT1, and HNRNPH3, in a sample of a subject to obtain a molecular signature indicative of a pigmented skin lesion characteristic. Additional methods and method details for characterizing melanoma are described elsewhere herein.

Described herein, in some embodiments, are methods for the identification of a skin lesion in a subject as comprising melanoma or not comprising melanoma, the methods comprising analyzing a nucleic acid molecule from one or more genes comprising: ACTB, ACTN4, B2M, LINC00518, CNN2, CMIP, EDNRB, GPM6B, KIT, MCOLN3, NAMPT, PPIA, SDCBP, TTC3, TMEM80, TRIB2, TYR, ANKRD11, CASC3, CCL18, CMPK2, CTBP2, DCT, EZR, FOS, FTL, HLA-A, HLA-C, HOXA9, IF16, IRF9, ISG15, KRT17, KTN1, MAFB, MAFK, MAL, MALAT1, MAP1B, MARCKS, MLANA, RNA00188, OTUB1, OVOS2, PKD1P1, PMEL, PRAME, PTPN14, SAT1, SDHA, SIRPA, SOX10, TFRC, TYRP1, UBE2B, or VIM, in a sample of the skin lesion, thereby identifying melanoma or not identifying melanoma in the skin lesion. In some embodiments, LINC00518 is analyzed. In some embodiments, the analyzed genes comprise LINC00518, TRIB2, KIT, SDCBP, TYR, NAMPT, ACTN4, EDNRB, GPM6B, CNN2, MCOLN3, PRAME, TMEM80, TTC3, and CMIP, and combinations thereof. In some embodiments, the analyzed genes comprise LINC00518, CMIP, ACTN4, TMEM80 and NAMPT and combinations thereof. In some embodiments, the one or more genes comprise LINC00518 and CMIP. In some embodiments, the combination of LINC00518 and CMIP is analyzed. In some embodiments, the one or more genes comprise LINC00518 and TMEM80. In some embodiments, the combination of LINC00518 and TMEM80 is analyzed. In some embodiments, the one of more genes comprise LINC00518 and ACTN4. In some embodiments, the combination of LINC00518 and ACTN4 are analyzed. In some embodiments, the nucleic acid molecule comprises RNA. In some embodiments, analyzing RNA comprises reverse transcription of the RNA to cDNA followed by amplification of the cDNA. In some embodiments, the amplified cDNA is detected using a detectably labeled probe. In some cases, the detectably labeled probe comprises a fluorophore and a quencher and comprises a nucleic acid sequence that is capable of hybridizing to the amplified cDNA. In some embodiments, analyzing the nucleic acid molecule comprises detecting one or more mutations in the nucleic acid sequence of the nucleic acid molecule. In some embodiments, the one or more mutations are selected from the group consisting of a substitution, a deletion, and an insertion. In some embodiments, the method further comprises amplifying the nucleic acid molecule obtained from the sample prior to analyzing. In some embodiments, the sample is obtained by applying an adhesive tape to a target area of skin in a manner sufficient to isolate the sample adhering to the adhesive tape. In some embodiments, the tape comprises a rubber adhesive on a polyurethane film. In some embodiments, about one to ten adhesive tapes or one to ten applications of a tape are applied and removed from the skin. In some embodiments, the method further comprises using the characterization to determine a treatment regimen. In some embodiments, the nucleic acid molecule, or an amplification product thereof, is applied to a microarray. In some embodiments, analyzing a nucleic acid molecule comprises detecting an expression profile. In some embodiments, the expression profile is detected using a microarray. In some embodiments, the sample is obtained from a biopsy taken at the site of the skin lesion or surrounding margin. In some embodiments, the method further comprises taking a biopsy of the target area of the skin. In some embodiments, the analyzing is performed in situ.

Disclosed herein, in some embodiments, is a method of distinguishing melanoma from dysplastic nevi or normal pigmented skin in a subject, comprising analyzing a nucleic acid molecule from one or more genes selected from: ACTB, ACTN4, B2M, LINC00518, CNN2, CMIP, EDNRB, GPM6B, KIT, MCOLN3, NAMPT, PPIA, SDCBP, TTC3, TMEM80, TRIB2, TYR, ANKRD11, CASC3, CCL18, CMPK2, CTBP2, DCT, EZR, FOS, FTL, HLA-A, HLA-C, HOXA9, IF16, IRF9, ISG15, KRT17, KTN1, MAFB, MAFK, MAL, MALAT1, MAP1B, MARCKS, MLANA, RNA00188, OTUB1, OVOS2, PKD1P1, PMEL, PRAME, PTPN14, SAT1, SDHA, SIRPA, SOX10, TFRC, TYRP1, UBE2B, and VIM, in a sample from the subject, thereby distinguishing melanoma from dysplastic nevi or normal pigmented skin in a subject. In some embodiments, the one or more genes comprise LINC00518, TRIB2, KIT, SDCBP, TYR, NAMPT, ACTN4, EDNRB, GPM6B, CNN2, MCOLN3, PRAME, TMEM80, TTC3, CMIP, or combinations thereof. In some embodiments, one or more genes is selected from: ACTN4, LINC00518, CMIP, TTC3, TMEM80, HLA-C, and PRAME, and combinations thereof. In some embodiments, two or more genes are selected from: ACTB, ACTN4, B2M, LINC00518, CNN2, CMIP, EDNRB, GPM6B, KIT, MCOLN3, NAMPT, PPIA, SDCBP, TTC3, TMEM80, TRIB2, TYR, ANKRD11, CASC3, CCL18, CMPK2, CTBP2, DCT, EZR, FOS, FTL, HLA-A, HLA-C, HOXA9, IF16, IRF9, ISG15, KRT17, KTN1, MAFB, MAFK, MAL, MALAT1, MAP1B, MARCKS, MLANA, RNA00188, OTUB1, OVOS2, PKD1P1, PMEL, PRAME, PTPN14, SAT1, SDHA, SIRPA, SOX10, TFRC, TYRP1, UBE2B, and VIM, and combinations thereof. In some embodiments, the two or more genes are selected from LINC00518, TRIB2, KIT, SDCBP, TYR, NAMPT, ACTN4, EDNRB, GPM6B, CNN2, MCOLN3, PRAME, TMEM80, TTC3, and CMIP, and combinations thereof. In some embodiments, the two or more genes is selected from: ACTN4, LINC00518, CMIP, TTC3, TMEM80, HLA-C, and PRAME. In some embodiments, the genes analyzed are selected from the group consisting of LINC00518, CMIP, ACTN4, TMEM80 and NAMPT and combinations thereof. In some embodiments, the genes analyzed are selected from the group consisting of ACTN4, LINC00518, CMIP, TTC3, TMEM80, HLA-C, and PRAME and combinations thereof. In some embodiments, LINC00518 is analyzed. In some embodiments, the two or more genes comprise LINC00518 and CMIP. In some embodiments, the combination of LINC00518 and CMIP is analyzed. In some embodiments, the two or more genes comprise LINC00518 and TMEM80. In some embodiments, the combination of LINC00518 and TMEM80 is analyzed. In some embodiments, the two or more genes comprise LINC00518 and ACTN4. In some embodiments, the combination of LINC00518 and ACTN4 are analyzed. In some embodiments, the two or more genes comprise LINC00518 and NAMPT. In some embodiments, the combination of LINC00518 and NAMPT are analyzed. In some embodiments, the two or more genes comprise LINC00518 and TTC3. In some embodiments, the combination of LINC00518 and TTC3 are analyzed. In some embodiments, the two or more genes comprise LINC00518 and HLA-C. In some embodiments, the combination of LINC00518 and HLA-C are analyzed. In some embodiments, the two or more genes comprise LINC00518 and PRAME. In some embodiments, the combination of LINC00518 and PRAME are analyzed. In some embodiments, the two or more genes comprise ACTN4, LINC00518 and PRAME, and combinations thereof. In some embodiments, the combination of ACTN4, LINC00518 and PRAME are analyzed. In some embodiments, the two or more genes comprise LINC00518 and TRIB2. In some embodiments, the two or more genes comprise LINC00518, CMIP and PRAME, and combinations thereof. In some embodiments, the combination of LINC00518, CMIP, and PRAME are analyzed. In some embodiments, the two or more genes comprise LINC00518 and GPM6B. In some embodiments, the combination of LINC00518 and GPM6B is analyzed. In some embodiments, the two or more genes comprise TRIB2 and NAMPT. In some embodiments, the combination of TRIB2 and NAMPT is analyzed. In some embodiments, the two or more genes comprise TRIB2 and KIT. In some embodiments, the combination of TRIB2 and KIT is analyzed. In some embodiments, the combination of TRIB2 and ACTN4 is analyzed. In some embodiments, the combination of TRIB2 and ACTN4 is analyzed.

Disclosed herein, in some embodiments, is a method of diagnosing or identifying melanoma in a subject, comprising analyzing a nucleic acid molecule from one or more genes in a melanoma diagnostic signature comprising ACTB, ACTN4, B2M, LINC00518, CNN2, CMIP, EDNRB, GPM6B, KIT, MCOLN3, NAMPT, PPIA, SDCBP, TTC3, TMEM80, TRIB2, TYR, ANKRD11, CASC3, CCL18, CMPK2, CTBP2, DCT, EZR, FOS, FTL, HLA-A, HLA-C, HOXA9, IF16, IRF9, ISG15, KRT17, KTN1, MAFB, MAFK, MAL, MALAT1, MAP1B, MARCKS, MLANA, RNA00188, OTUB1, OVOS2, PKD1P1, PMEL, PRAME, PTPN14, SAT1, SDHA, SIRPA, SOX10, TFRC, TYRP1, UBE2B, VIM, or any combination thereof, in a sample from the subject, thereby diagnosing the presence or absence of melanoma in a subject. In some embodiments, a melanoma diagnostic signature comprises LINC00518, TRIB2, KIT, SDCBP, TYR, NAMPT, ACTN4, EDNRB, GPM6B, CNN2, MCOLN3, PRAME, TMEM80, TTC3, CMIP, or combinations thereof. In some embodiments, a melanoma diagnostic signature comprises ACTN4, LINC00518, CMIP, TTC3, TMEM80, HLA-C, PRAME, or combinations thereof. In some embodiments, a melanoma diagnostic signature comprises ACTB, ACTN4, B2M, LINC00518, CNN2, CMIP, EDNRB, GPM6B, KIT, MCOLN3, NAMPT, PPIA, SDCBP, TTC3, TMEM80, TRIB2, TYR, ANKRD11, CASC3, CCL18, CMPK2, CTBP2, DCT, EZR, FOS, FTL, HLA-A, HLA-C, HOXA9, IF16, IRF9, ISG15, KRT17, KTN1, MAFB, MAFK, MAL, MALAT1, MAP1B, MARCKS, MLANA, RNA00188, OTUB1, OVOS2, PKD1P1, PMEL, PRAME, PTPN14, SAT1, SDHA, SIRPA, SOX10, TFRC, TYRP1, UBE2B, VIM, or combinations thereof. In some embodiments, the a melanoma diagnostic signature comprises two or more genes selected from: LINC00518, TRIB2, KIT, SDCBP, TYR, NAMPT, ACTN4, EDNRB, GPM6B, CNN2, MCOLN3, PRAME, TMEM80, TTC3, and CMIP, and combinations thereof. In some embodiments, the two or more genes is selected from: ACTN4, LINC00518, CMIP, TTC3, TMEM80, HLA-C, and PRAME. In some embodiments, the genes analyzed are selected from the group consisting of LINC00518, CMIP, PRAME, and combinations thereof. In some embodiments, the genes analyzed are selected from the group consisting of ACTN4, LINC00518, CMIP, TTC3, TMEM80, HLA-C, and PRAME and combinations thereof. In some embodiments, LINC00518 is analyzed. In some embodiments, the two or more genes comprise LINC00518 and CMIP. In some embodiments, the combination of LINC00518 and CMIP is analyzed. In some embodiments, the two or more genes comprise LINC00518 and TMEM80, and combinations thereof. In some embodiments, the combination of LINC00518 and TMEM80 is analyzed. In some embodiments, the two or more genes comprise LINC00518 and ACTN4, and combinations thereof. In some embodiments, the combination of LINC00518 and ACTN4 are analyzed. In some embodiments, the two or more genes comprise LINC00518 and NAMPT, and combinations thereof. In some embodiments, the combination of LINC00518 and NAMPT are analyzed. In some embodiments, the two or more genes comprise LINC00518 and TTC3, and combinations thereof. In some embodiments, the combination of LINC00518 and TTC3 are analyzed. In some embodiments, the two or more genes comprise LINC00518 and HLA-C, and combinations thereof. In some embodiments, the combination of LINC00518 and HLA-C are analyzed. In some embodiments, the two or more genes comprise LINC00518 and PRAME, and combinations thereof. In some embodiments, the combination of LINC00518 and PRAME are analyzed. In some embodiments, the two or more genes comprise CMIP, LINC00518 and PRAME, and combinations thereof. In some embodiments, the combination of ACTN4, LINC00518 and PRAME are analyzed. In some embodiments, the two or more genes comprise LINC00518 and TRIB2, and combinations thereof. In some embodiments, the two or more genes comprise LINC00518 and TRIB2, and combinations thereof. In some embodiments, the two or more genes comprise LINC00518 and GPM6B, and combinations thereof. In some embodiments, the combination of LINC00518 and GPM6B is analyzed. In some embodiments, the two or more genes comprise TRIB2 and NAMPT, and combinations thereof. In some embodiments, the combination of TRIB2 and NAMPT is analyzed. In some embodiments, the two or more genes comprise TRIB2 and KIT, and combinations thereof. In some embodiments, the combination of TRIB2 and KIT is analyzed. In some embodiments, the combination of TRIB2 and ACTN4 is analyzed. In some embodiments, the combination of TRIB2 and ACTN4 is analyzed.

Disclosed herein, in some embodiments, is a system for identifying a presence or absence of melanoma in a skin lesion of a subject, comprising analyzing a nucleic acid molecule from one or more genes in a melanoma diagnostic signature comprising: ACTB, ACTN4, B2M, LINC00518, CNN2, CMIP, EDNRB, GPM6B, KIT, MCOLN3, NAMPT, PPIA, SDCBP, TTC3, TMEM80, TRIB2, TYR, ANKRD11, CASC3, CCL18, CMPK2, CTBP2, DCT, EZR, FOS, FTL, HLA-A, HLA-C, HOXA9, IF16, IRF9, ISG15, KRT17, KTN1, MAFB, MAFK, MAL, MALAT1, MAP1B, MARCKS, MLANA, RNA00188, OTUB1, OVOS2, PKD1P1, PMEL, PRAME, PTPN14, SAT1, SDHA, SIRPA, SOX10, TFRC, TYRP1, UBE2B, VIM, or a combination thereof, in a sample of the skin lesion, thereby identifying the skin lesion as comprising or not comprising melanoma. In some embodiments, the one or more genes are selected from LINC00518, TRIB2, KIT, SDCBP, TYR, NAMPT, ACTN4, EDNRB, GPM6B, CNN2, MCOLN3, PRAME, TMEM80, TTC3, and CMIP, and combinations thereof. In some embodiments, one or more genes is selected from: ACTN4, LINC00518, CMIP, TTC3, TMEM80, HLA-C, and PRAME, and combinations thereof. In some embodiments, two or more genes are selected from: ACTB, ACTN4, B2M, LINC00518, CNN2, CMIP, EDNRB, GPM6B, KIT, MCOLN3, NAMPT, PPIA, SDCBP, TTC3, TMEM80, TRIB2, TYR, ANKRD11, CASC3, CCL18, CMPK2, CTBP2, DCT, EZR, FOS, FTL, HLA-A, HLA-C, HOXA9, IF16, IRF9, ISG15, KRT17, KTN1, MAFB, MAFK, MAL, MALAT1, MAP1B, MARCKS, MLANA, RNA00188, OTUB1, OVOS2, PKD1P1, PMEL, PRAME, PTPN14, SAT1, SDHA, SIRPA, SOX10, TFRC, TYRP1, UBE2B, and VIM, and combinations thereof. In some embodiments, the two or more genes are selected from LINC00518, TRIB2, KIT, SDCBP, TYR, NAMPT, ACTN4, EDNRB, GPM6B, CNN2, MCOLN3, PRAME, TMEM80, TTC3, and CMIP, and combinations thereof. In some embodiments, the two or more genes is selected from: ACTN4, LINC00518, CMIP, TTC3, TMEM80, HLA-C, and PRAME. In some embodiments, the genes analyzed are selected from the group consisting of LINC00518, CMIP, ACTN4, TMEM80 and NAMPT and combinations thereof. In some embodiments, the genes analyzed are selected from the group consisting of ACTN4, LINC00518, CMIP, TTC3, TMEM80, HLA-C, and PRAME and combinations thereof. In some embodiments, LINC00518 is analyzed. In some embodiments, the two or more genes comprise LINC00518 and CMIP, and combinations thereof. In some embodiments, the combination of LINC00518 and CMIP is analyzed. In some embodiments, the two or more genes comprise LINC00518 and TMEM80, and combinations thereof. In some embodiments, the combination of LINC00518 and TMEM80 is analyzed. In some embodiments, the two or more genes comprise LINC00518 and ACTN4, and combinations thereof. In some embodiments, the combination of LINC00518 and ACTN4 are analyzed. In some embodiments, the two or more genes comprise LINC00518 and NAMPT, and combinations thereof. In some embodiments, the combination of LINC00518 and NAMPT are analyzed. In some embodiments, the two or more genes comprise LINC00518 and TTC3, and combinations thereof. In some embodiments, the combination of LINC00518 and TTC3 are analyzed. In some embodiments, the two or more genes comprise LINC00518 and HLA-C, and combinations thereof. In some embodiments, the combination of LINC00518 and HLA-C are analyzed. In some embodiments, the two or more genes comprise LINC00518 and PRAME, and combinations thereof. In some embodiments, the combination of LINC00518 and PRAME are analyzed. In some embodiments, the two or more genes comprise CMIP, LINC00518 and PRAME, and combinations thereof. In some embodiments, the combination of CMIP, LINC00518 and PRAME are analyzed. In some embodiments, the two or more genes comprise LINC00518 and TRIB2, and combinations thereof. In some embodiments, the two or more genes comprise LINC00518 and TRIB2, and combinations thereof. In some embodiments, the two or more genes comprise LINC00518 and GPM6B, and combinations thereof. In some embodiments, the combination of LINC00518 and GPM6B is analyzed. In some embodiments, the two or more genes comprise TRIB2 and NAMPT, and combinations thereof. In some embodiments, the combination of TRIB2 and NAMPT is analyzed. In some embodiments, the two or more genes comprise TRIB2 and KIT, and combinations thereof. In some embodiments, the combination of TRIB2 and KIT is analyzed. In some embodiments, the combination of TRIB2 and ACTN4 is analyzed. In some embodiments, the combination of TRIB2 and ACTN4 is analyzed.

Disclosed herein, in some embodiments, is a system for identifying the presence or absence of melanoma in a skin lesion in a subject in a subject, comprising: (a) a computer processing device, optionally connected to a computer network; and (b) a software module executed by the computer processing device to analyzing a nucleic acid molecule from one or more genes selected from: ACTB, ACTN4, B2M, LINC00518, CNN2, CMIP, EDNRB, GPM6B, KIT, MCOLN3, NAMPT, PPIA, SDCBP, TTC3, TMEM80, TRIB2, TYR, ANKRD11, CASC3, CCL18, CMPK2, CTBP2, DCT, EZR, FOS, FTL, HLA-A, HLA-C, HOXA9, IF16, IRF9, ISG15, KRT17, KTN1, MAFB, MAFK, MAL, MALAT1, MAP1B, MARCKS, MLANA, RNA00188, OTUB1, OVOS2, PKD1P1, PMEL, PRAME, PTPN14, SAT1, SDHA, SIRPA, SOX10, TFRC, TYRP1, UBE2B, in a sample from a skin lesion, to a standard or control. In some embodiments, the one or more genes are selected from LINC00518, TRIB2, KIT, SDCBP, TYR, NAMPT, ACTN4, EDNRB, GPM6B, CNN2, MCOLN3, PRAME, TMEM80, TTC3, and CMIP, and combinations thereof. In some embodiments, one or more genes is selected from: ACTN4, LINC00518, CMIP, TTC3, TMEM80, HLA-C, and PRAME, and combinations thereof. In some embodiments, two or more genes are selected from: ACTB, ACTN4, B2M, LINC00518, CNN2, CMIP, EDNRB, GPM6B, KIT, MCOLN3, NAMPT, PPIA, SDCBP, TTC3, TMEM80, TRIB2, TYR, ANKRD11, CASC3, CCL18, CMPK2, CTBP2, DCT, EZR, FOS, FTL, HLA-A, HLA-C, HOXA9, IF16, IRF9, ISG15, KRT17, KTN1, MAFB, MAFK, MAL, MALAT1, MAP1B, MARCKS, MLANA, RNA00188, OTUB1, OVOS2, PKD1P1, PMEL, PRAME, PTPN14, SAT1, SDHA, SIRPA, SOX10, TFRC, TYRP1, UBE2B, and VIM, and combinations thereof. In some embodiments, the two or more genes are selected from LINC00518, TRIB2, KIT, SDCBP, TYR, NAMPT, ACTN4, EDNRB, GPM6B, CNN2, MCOLN3, PRAME, TMEM80, TTC3, and CMIP, and combinations thereof. In some embodiments, the two or more genes is selected from: ACTN4, LINC00518, CMIP, TTC3, TMEM80, HLA-C, and PRAME. In some embodiments, the genes analyzed are selected from the group consisting of LINC00518, CMIP, ACTN4, TMEM80 and NAMPT and combinations thereof. In some embodiments, the genes analyzed are selected from the group consisting of ACTN4, LINC00518, CMIP, TTC3, TMEM80, HLA-C, and PRAME and combinations thereof. In some embodiments, LINC00518 is analyzed. In some embodiments, the two or more genes comprise LINC00518 and CMIP, and combinations thereof. In some embodiments, the combination of LINC00518 and CMIP is analyzed. In some embodiments, the two or more genes comprise LINC00518 and TMEM80, and combinations thereof. In some embodiments, the combination of LINC00518 and TMEM80 is analyzed. In some embodiments, the two or more genes comprise LINC00518 and ACTN4, and combinations thereof. In some embodiments, the combination of LINC00518 and ACTN4 are analyzed. In some embodiments, the two or more genes comprise LINC00518 and NAMPT, and combinations thereof. In some embodiments, the combination of LINC00518 and NAMPT are analyzed. In some embodiments, the two or more genes comprise LINC00518 and TTC3, and combinations thereof. In some embodiments, the combination of LINC00518 and TTC3 are analyzed. In some embodiments, the two or more genes comprise LINC00518 and HLA-C, and combinations thereof. In some embodiments, the combination of LINC00518 and HLA-C are analyzed. In some embodiments, the two or more genes comprise LINC00518 and PRAME, and combinations thereof. In some embodiments, the combination of LINC00518 and PRAME are analyzed. In some embodiments, the two or more genes comprise CMIP, LINC00518 and PRAME, and combinations thereof. In some embodiments, the combination of CMIP, LINC00518 and PRAME are analyzed. In some embodiments, the two or more genes comprise LINC00518 and TRIB2, and combinations thereof. In some embodiments, the two or more genes comprise LINC00518 and TRIB2, and combinations thereof. In some embodiments, the two or more genes comprise LINC00518 and GPM6B, and combinations thereof. In some embodiments, the combination of LINC00518 and GPM6B is analyzed. In some embodiments, the two or more genes comprise TRIB2 and NAMPT, and combinations thereof. In some embodiments, the combination of TRIB2 and NAMPT is analyzed. In some embodiments, the two or more genes comprise TRIB2 and KIT, and combinations thereof. In some embodiments, the combination of TRIB2 and KIT is analyzed. In some embodiments, the combination of TRIB2 and ACTN4 is analyzed. In some embodiments, the combination of TRIB2 and ACTN4 is analyzed.

In some embodiments, the analyzing of one or more nucleic acid molecule from one or more genes comprises any analysis method described elsewhere herein. In some embodiments, analyzing a nucleic acid molecule comprises determining an expression level of the nucleic acid molecule. In some embodiments, an expression level is determined by hybridizing a probe comprising a nucleic acid sequence complementary to the nucleic acid molecule. In some embodiments, the probe is a detectably labeled probe. In some embodiments, the nucleic acid molecule is a RNA molecule whose expression level is determined by reverse transcription of the RNA molecule to cDNA, followed by amplification of the cDNA with a detectably labeled probe in a real time quantitative PCR.

In some embodiments, analyzing a nucleic acid molecule comprises detecting the presence or absence or the nucleic acid molecule in a sample.

In some embodiments, nucleic acid expression products expressed from one or more genes in a melanoma diagnostic signature comprising: ACTB, ACTN4, B2M, LINC00518, CNN2, CMIP, EDNRB, GPM6B, KIT, MCOLN3, NAMPT, PPIA, SDCBP, TTC3, TMEM80, TRIB2, TYR, ANKRD11, CASC3, CCL18, CMPK2, CTBP2, DCT, EZR, FOS, FTL, HLA-A, HLA-C, HOXA9, IF16, IRF9, ISG15, KRT17, KTN1, MAFB, MAFK, MAL, MALAT1, MAP1B, MARCKS, MLANA, RNA00188, OTUB1, OVOS2, PKD1P1, PMEL, PRAME, PTPN14, SAT1, SDHA, SIRPA, SOX10, TFRC, TYRP1, UBE2B, VIM, or a combination thereof, are detected for their presence or absence in a biological sample suspected of comprising the nucleic acid expression products expressed from one or more genes in the melanoma diagnostic signature. In some embodiments, the presence or absence of one or more nucleic acid expression products from one or more of the genes in the melanoma diagnostic signature is indicative of the presence of melanoma in the biological sample. In some embodiments, the presence or absence of nucleic acid expression products from one or more of the genes in the melanoma diagnostic signature is indicative of melanoma in situ. In some embodiments, the presence or absence of nucleic acid expression products from one or more of the genes in the melanoma diagnostic signature is indicative of invasive melanoma. In some embodiments, detecting the presence or absence of a nucleic acid expressed from a gene in the melanoma diagnostic signature comprises applying one or more probes to the biological sample, wherein each probe is capable of hybridizing to one or more nucleic acids expressed from one or more genes in the melanoma diagnostic signature. For example, each probe is configured to bind to each nucleic acid expression product of a gene in the melanoma diagnostic signature. In some cases, the biological sample is tissue, blood, serum, plasma or urine sample. In some cases, the biological sample comprises a skin sample. In some cases, the biological sample comprises a skin lesion. In some cases, the biological sample comprises one or more melanoma cells.

In some embodiments, expression levels of nucleic acids expressed from one or more genes in a melanoma diagnostic signature comprising ACTB, ACTN4, B2M, LINC00518, CNN2, CMIP, EDNRB, GPM6B, KIT, MCOLN3, NAMPT, PPIA, SDCBP, TTC3, TMEM80, TRIB2, TYR, ANKRD11, CASC3, CCL18, CMPK2, CTBP2, DCT, EZR, FOS, FTL, HLA-A, HLA-C, HOXA9, IF16, IRF9, ISG15, KRT17, KTN1, MAFB, MAFK, MAL, MALAT1, MAP1B, MARCKS, MLANA, RNA00188, OTUB1, OVOS2, PKD1P1, PMEL, PRAME, PTPN14, SAT1, SDHA, SIRPA, SOX10, TFRC, TYRP1, UBE2B, VIM, or a combination thereof are determined in a biological sample suspected of or known to comprise nucleic acid expression products of the melanoma diagnostic signature. In some embodiments, determining the expression level of a nucleic acid expressed from a gene in the melanoma diagnostic signature comprises applying one or more probes to the biological sample, wherein each probe is capable of hybridizing to one or more nucleic acids expressed from one or more genes in the melanoma diagnostic signature. For example, each probe is configured to bind to each nucleic acid expression product of a gene in the melanoma diagnostic signature. In some cases, determining the expression level of a nucleic acid comprises performing PCR on the biological sample to amplify the nucleic acids. In some cases, determining the expression level of a nucleic acid comprises performing quantitative PCR on the biological sample. In some cases, determining the expression level of a nucleic acid comprises performing real-time quantitative PCR on the biological sample. In some embodiments, the expression level of one or more nucleic acid expression products is compared a known expression level of the one or more nucleic acid expression products in a reference sample. In some cases, the reference sample comprises a sample comprising melanoma. In some cases, the reference sample comprises a sample that does not comprise melanoma.

In some embodiments, a decrease or increase in expression level of a nucleic acid expressed from a gene in a melanoma diagnostic signature of a subject compared to a reference expression level of a corresponding nucleic acid expressed from the gene in the melanoma diagnostic signature of a reference sample is indicative of the subject comprising melanoma. In some embodiments, a lesion of the subject comprises melanoma. In some embodiments, if the gene is LINC00518 and the expression of LINC00518 in the subject is greater than in a reference sample that does not comprise melanoma, then the subject is indicated to comprise melanoma. In some embodiments, if the gene is PRAME and the expression of PRAME in the subject is greater than in a reference sample that does not comprise melanoma, then the subject is indicated to comprise melanoma. In some embodiments, if the gene is CMIP and the expression of CMIP in the subject is lower or absent than in a reference sample that does not comprise melanoma, then the subject is indicated to comprise melanoma.

### Systems

Disclosed herein, in some embodiments, is a system for characterizing a pigmented skin lesion in a subject, comprising analyzing genes or gene products expressed from 1, 2, 3, 4, 5, 6, 7, 8, 9, 10, 11, 12, 13, 14, 15, 16, 17, 18, 19, or 20 of the following genes WFDC3, SLC16A6, DUSP4, PPAP2A, NDUFAF4, BCL2A1, DMXL2, SMG1, SLC2A3, VEGFA, BEX4, SH3BP5, RAB27A, FEM1A, CYTH3, IRGQ, EFNA5, NAMPT, PNPT1, and HNRNPH3, in a sample of a subject to obtain a molecular signature indicative of a pigmented skin lesion characteristic. In some embodiments, 1, 2, 3, 4, 5, or 6 of the following genes or gene expression products WFDC3, SLC16A6, DUSP4, PPAP2A, NDUFAF4 and BCL2A1 are analyzed. The system is configured to implement the methods described in this disclosure, including, but limited to, analyzing genes or gene expression products of a subject to obtain a molecular signature and/or characterize a pigmented skin lesion.

In some embodiments, disclosed herein is a system for characterizing a pigmented skin lesion in a subject, comprising: (a) a computer processing device, optionally connected to a computer network; and (b) a software module executed by the computer processing device to analyze a gene or gene expression product from one or more of the following genes WFDC3, SLC16A6, DUSP4, PPAP2A, NDUFAF4, BCL2A1, DMXL2, SMG1, SLC2A3, VEGFA, BEX4, SH3BP5, RAB27A, FEM1A, CYTH3, IRGQ, EFNA5, NAMPT, PNPT1, and HNRNPH3 in a sample from a subject. In some instances, the system comprises a central processing unit (CPU), memory (e.g., random access memory, flash memory), electronic storage unit, computer program, communication interface to communicate with one or more other systems, and any combination thereof. In some instances, the system is coupled to a computer network, for example, the Internet, intranet, and/or extranet that is in communication with the Internet, a telecommunication, or data network. In some embodiments, the system comprises a storage unit to store data and information regarding any aspect of the methods described in this disclosure. Various aspects of the system are a product or article or manufacture.

One feature of a computer program includes a sequence of instructions, executable in the digital processing device's CPU, written to perform a specified task. In some embodiments, computer readable instructions are implemented as program modules, such as functions, features, Application Programming Interfaces (APIs), data structures, and the like, that perform particular tasks or implement particular abstract data types. In light of the disclosure provided herein, those of skill in the art will recognize that a computer program may be written in various versions of various languages.

The functionality of the computer readable instructions are combined or distributed as desired in various environments. In some instances, a computer program comprises one sequence of instructions or a plurality of sequences of instructions. A computer program may be provided from one location. A computer program may be provided from a plurality of locations. In some embodiment, a computer program includes one or more software modules. In some embodiments, a computer program includes, in part or in whole, one or more web applications, one or more mobile applications, one or more standalone applications, one or more web browser plug-ins, extensions, add-ins, or add-ons, or combinations thereof.

### Web Application

In some embodiments, a computer program includes a web application. In light of the disclosure provided herein, those of skill in the art will recognize that a web application may utilize one or more software frameworks and one or more database systems. A web application, for example, is created upon a software framework such as Microsoft® .NET or Ruby on Rails (RoR). A web application, in some instances, utilizes one or more database systems including, by way of non-limiting examples, relational, non-relational, feature oriented, associative, and XML database systems. Suitable relational database systems include, by way of non-limiting examples, Microsoft® SQL Server, mySQL™, and Oracle®. Those of skill in the art will also recognize that a web application may be written in one or more versions of one or more languages. In some embodiments, a web application is written in one or more markup languages, presentation definition languages, client-side scripting languages, server-side coding languages, database query languages, or combinations thereof. In some embodiments, a web application is written to some extent in a markup language such as Hypertext Markup Language (HTML), Extensible Hypertext Markup Language (XHTML), or eXtensible Markup Language (XML). In some embodiments, a web application is written to some extent in a presentation definition language such as Cascading Style Sheets (CSS). In some embodiments, a web application is written to some extent in a client-side scripting language such as Asynchronous Javascript and XML (AJAX), Flash® Actionscript, Javascript, or Silverlight®. In some embodiments, a web application is written to some extent in a server-side coding language such as Active Server Pages (ASP), ColdFusion®, Perl, Java™, JavaServer Pages (JSP), Hypertext Preprocessor (PHP), Python™, Ruby, Tcl, Smalltalk, WebDNA®, or Groovy. In some embodiments, a web application is written to some extent in a database query language such as Structured Query Language (SQL). A web application may integrate enterprise server products such as IBM® Lotus Domino®. A web application may include a media player element. A media player element may utilize one or more of many suitable multimedia technologies including, by way of non-limiting examples, Adobe® Flash®, HTML 5, Apple® QuickTime®, Microsoft® Silverlight®, Java™, and Unity®.

### Mobile Application

In some instances, a computer program includes a mobile application provided to a mobile digital processing device. The mobile application may be provided to a mobile digital processing device at the time it is manufactured. The mobile application may be provided to a mobile digital processing device via the computer network described herein.

A mobile application is created by techniques known to those of skill in the art using hardware, languages, and development environments known to the art. Those of skill in the art will recognize that mobile applications may be written in several languages. Suitable programming languages include, by way of non-limiting examples, C, C++, C#, Featureive-C, Java™, Javascript, Pascal, Feature Pascal, Python™, Ruby, VB.NET, WML, and XHTML/HTML with or without CSS, or combinations thereof.

Suitable mobile application development environments are available from several sources. Commercially available development environments include, by way of non-limiting examples, AirplaySDK, alcheMo, Appcelerator®, Celsius, Bedrock, Flash Lite, .NET Compact Framework, Rhomobile, and WorkLight Mobile Platform. Other development environments may be available without cost including, by way of non-limiting examples, Lazarus, MobiFlex, MoSync, and Phonegap. Also, mobile device manufacturers distribute software developer kits including, by way of non-limiting examples, iPhone and iPad (iOS) SDK, Android™ SDK, BlackBerry® SDK, BREW SDK, Palm® OS SDK, Symbian SDK, webOS SDK, and Windows® Mobile SDK.

Those of skill in the art will recognize that several commercial forums are available for distribution of mobile applications including, by way of non-limiting examples, Apple® App Store, Android™ Market, BlackBerry® App World, App Store for Palm devices, App Catalog for webOS, Windows® Marketplace for Mobile, Ovi Store for Nokia® devices, Samsung® Apps, and Nintendo® DSi Shop.

### Standalone Application

In some embodiments, a computer program includes a standalone application, which is a program that may be run as an independent computer process, not an add-on to an existing process, e.g., not a plug-in. Those of skill in the art will recognize that standalone applications are sometimes compiled. In some instances, a compiler is a computer program(s) that transforms source code written in a programming language into binary feature code such as assembly language or machine code. Suitable compiled programming languages include, by way of non-limiting examples, C, C++, Featureive-C, COBOL, Delphi, Eiffel, Java™, Lisp, Python™, Visual Basic, and VB .NET, or combinations thereof. Compilation may be often performed, at least in part, to create an executable program. In some instances, a computer program includes one or more executable complied applications.

### Web Browser Plug-in

A computer program, in some aspects, includes a web browser plug-in. In computing, a plug-in, in some instances, is one or more software components that add specific functionality to a larger software application. Makers of software applications may support plug-ins to enable third-party developers to create abilities which extend an application, to support easily adding new features, and to reduce the size of an application. When supported, plug-ins enable customizing the functionality of a software application. For example, plug-ins are commonly used in web browsers to play video, generate interactivity, scan for viruses, and display particular file types. Those of skill in the art will be familiar with several web browser plug-ins including, Adobe® Flash® Player, Microsoft® Silverlight®, and Apple® QuickTime®. The toolbar may comprise one or more web browser extensions, add-ins, or add-ons. The toolbar may comprise one or more explorer bars, tool bands, or desk bands.

In view of the disclosure provided herein, those of skill in the art will recognize that several plug-in frameworks are available that enable development of plug-ins in various programming languages, including, by way of non-limiting examples, C++, Delphi, Java™, PHP, Python™, and VB .NET, or combinations thereof.

In some embodiments, Web browsers (also called Internet browsers) are software applications, designed for use with network-connected digital processing devices, for retrieving, presenting, and traversing information resources on the World Wide Web. Suitable web browsers include, by way of non-limiting examples, Microsoft® Internet Explorer®, Mozilla® Firefox®, Google® Chrome, Apple® Safari®, Opera Software® Opera®, and KDE Konqueror. The web browser, in some instances, is a mobile web browser. Mobile web browsers (also called mircrobrowsers, mini-browsers, and wireless browsers) may be designed for use on mobile digital processing devices including, by way of non-limiting examples, handheld computers, tablet computers, netbook computers, subnotebook computers, smartphones, music players, personal digital assistants (PDAs), and handheld video game systems. Suitable mobile web browsers include, by way of non-limiting examples, Google® Android® browser, RIM BlackBerry® Browser, Apple® Safari®, Palm® Blazer, Palm® WebOS® Browser, Mozilla® Firefox® for mobile, Microsoft® Internet Explorer® Mobile, Amazon® Kindle® Basic Web, Nokia® Browser, Opera Software® Opera® Mobile, and Sony® PSP™ browser.

### Software Modules

The medium, method, and system disclosed herein comprise one or more softwares, servers, and database modules, or use of the same. In view of the disclosure provided herein, software modules may be created by techniques known to those of skill in the art using machines, software, and languages known to the art. The software modules disclosed herein may be implemented in a multitude of ways. In some embodiments, a software module comprises a file, a section of code, a programming feature, a programming structure, or combinations thereof. A software module may comprise a plurality of files, a plurality of sections of code, a plurality of programming features, a plurality of programming structures, or combinations thereof. By way of non-limiting examples, the one or more software modules comprises a web application, a mobile application, and/or a standalone application. Software modules may be in one computer program or application. Software modules may be in more than one computer program or application. Software modules may be hosted on one machine. Software modules may be hosted on more than one machine. Software modules may be hosted on cloud computing platforms. Software modules may be hosted on one or more machines in one location. Software modules may be hosted on one or more machines in more than one location.

### Databases

The medium, method, and system disclosed herein comprise one or more databases, or use of the same. In view of the disclosure provided herein, those of skill in the art will recognize that many databases are suitable for storage and retrieval of geologic profile, operator activities, division of interest, and/or contact information of royalty owners. Suitable databases include, by way of non-limiting examples, relational databases, non-relational databases, feature oriented databases, feature databases, entity-relationship model databases, associative databases, and XML databases. In some embodiments, a database is internet-based. In some embodiments, a database is web-based. In some embodiments, a database is cloud computing-based. A database may be based on one or more local computer storage devices.

### Data Transmission

The subject matter described herein, including methods for obtaining and analyzing a molecular signature from a subject having a pigmented skin lesion, methods for obtaining a pigmented skin lesion, corresponding transmission of data, in certain aspects, are configured to be performed in one or more facilities at one or more locations. Facility locations are not limited by country and include any country or territory. In some instances, one or more steps for obtaining a molecular signature from a sample are performed in a different country than another step of the method. In some instances, one or more steps for obtaining a sample are performed in a different country than one or more steps for obtaining a molecular signature from a sample. In some embodiments, one or more method steps involving a computer system are performed in a different country than another step of the methods provided herein. In some embodiments, data processing and analyses are performed in a different country or location than one or more steps of the methods described herein. In some embodiments, one or more articles, products, or data are transferred from one or more of the facilities to one or more different facilities for analysis or further analysis. An article includes, but is not limited to, one or more components obtained from the tape stripping methods such as the adhesive tape, isolated cellular material obtained from an adhesive tape, processed cellular material, data, and any article or product disclosed herein as an article or product. Processed cellular material includes, but is not limited to, cDNA reverse transcribed from RNA, amplified RNA, amplified cDNA, sequenced DNA, isolated and/or purified RNA, isolated and/or purified DNA, and isolated and/or purified polypeptide. Data includes, but is not limited to, information regarding the gene expression profile of one or more target genes, information regarding a gene sequence profile signature, information regarding a protein sequence profile, information regarding the characteristic of a pigmented skin lesion (e.g., non-melanoma, melanoma in situ, invasive melanoma, stage 1 melanoma, stage 2 melanoma, stage 3 melanoma, stage 4 melanoma), and any data produced by the methods disclosed herein. In some embodiments of the methods and systems described herein, the analysis is performed and a subsequent data transmission step will convey or transmit the results of the analysis. Information regarding a pigmented skin lesion includes, but is not limited to, identification of melanoma, likelihood of treatment success for a subject having melanoma, identification of progression of a melanoma, identification of melanoma in situ, identification of invasive melanoma, and identification of a melanoma stage (e.g., 0, 1, 2, 3, 4).

In some embodiments, any step of any method described herein is performed by a software program or module on a computer. In additional or further embodiments, data from any step of any method described herein is transferred to and from facilities located within the same or different countries, including analysis performed in one facility in a particular location and the data shipped to another location or directly to an individual in the same or a different country. In additional or further embodiments, data from any step of any method described herein (including characterization of melanoma in situ and/or invasive melanoma, information regarding cellular material such as DNA, RNA, and protein as well as transformed data, e.g. a molecular signature, from cellular material) is transferred to and/or received from a facility located within the same or different countries, including analysis of a data input, such as cellular material, performed in one facility in a particular location and corresponding data transmitted to another location, or directly to an individual, such as data related to the diagnosis, prognosis, responsiveness to therapy, or the like, in the same or different location or country.

### Applications of the Methods

Methods provided herein which obtain cellular material comprising genes or gene expression products from an epidermal sample of a pigmented skin lesion have utility not only in characterizing the pigmented skin lesion, but also in diagnosing melanoma, prognosing melanoma, as well as monitoring a response of a subject with melanoma to treatment. In some embodiments, these methods are used to characterize a lesion as being melanoma, melanoma in situ, and/or invasive melanoma. In some embodiments, these methods identify subjects who will respond positively to a given treatment for melanoma. In some embodiments, the progression of melanoma in a pigmented skin lesion is monitored. For example, by comparing the gene or gene expression profile prior to treatment with the gene or gene expression profile after treatment. In one embodiment, the method characterizes a pigmented lesion as melanoma in situ or invasive melanoma based on analysis of 1, 2, 3, 4, 5, 6, 7, 8, 9, 10, 11, 12, 13, 14, 15, 16, 17, 18, 19 or 20 of the following genes or gene expression products WFDC3, SLC16A6, DUSP4, PPAP2A, NDUFAF4, BCL2A1, DMXL2, SMG1, SLC2A3, VEGFA, BEX4, SH3BP5, RAB27A, FEM1A, CYTH3, IRGQ, EFNA5, NAMPT, PNPT1, and HNRNPH3.

It is known that in many cases, by the time a diagnosis of melanoma is established in a subject, metastasis has already occurred since melanomas contain multiple cell populations characterized by diverse growth rates, karyotypes, cell-surface properties, antigenicity, immunogenicity, invasion, metastasis, and sensitivity to cytotoxic drugs or biologic agents. Thus, the present disclosure, in some embodiments, is used to characterize cancer of an organ as having metastasized from melanoma.

In a related aspect, the methods of the present disclosure can also be useful for determining an appropriate treatment regimen for a subject having melanoma. Thus, the methods of the disclosure are useful for providing a means for practicing personalized medicine, wherein treatment is tailored to a subject based on the particular characteristics of the melanoma or pigmented skin lesion in the subject. The method can be practiced, for example, by first determining whether the pigmented skin lesion is melanoma or solar lentigo, as described above.

The sample of cells examined according to the present method can be obtained from the subject to be treated, or can be cells of an established cancer cell line of the same type as that of the subject. In one aspect, the established cell line can be one of a panel of such cell lines, wherein the panel can include different cell lines of the same type of disease and/or different cell lines of different diseases associated with expression of the genes of interest. Such a panel of cell lines can be useful, for example, to practice the present method when only a small number of cells can be obtained from the subject to be treated, thus providing a surrogate sample of the subject's cells, and also can be useful to include as control samples in practicing the present methods.

Once disease and/or pigmented skin lesion characterization is established and a treatment protocol is initiated, the methods of the disclosure, in some instances, is repeated on a regular basis to monitor the molecular signature of the genes or gene expression products of interest in the subject. The results obtained from successive assays may be used to show the efficacy of treatment over a period ranging from several days to months. Accordingly, another aspect of the disclosure is directed to methods for monitoring a therapeutic regimen for treating a subject having skin cancer. A comparison of the gene expression profile or mutations in the nucleic acid sequence of the nucleic acid molecule prior to and during therapy will be indicative of the efficacy of the therapy. Therefore, one skilled in the art will be able to recognize and adjust the therapeutic approach as needed.

Treatment regimens for melanoma include chemotherapeutic agents, radiation, anti-angiogenic compounds, or other agents for treating cancer in combination with immunization strategies. Other suitable treatments for melanoma include, for example, surgery, adjuvant radiation therapy, adjuvant interferon alfa-2b, corticosteroids, systemic therapy, dacarbazine, combination chemotherapies, such as cisplatin, carmustine, dacarbazine, and tamoxifen. In some embodiments, the melanoma is treated with Yervoy, nivolumab, MPDL3280, or a combination thereof. Other suitable treatments are known in the art.

The efficacy of a therapeutic regimen for treating a cancer over time can be identified by an absence of symptoms or clinical signs of the particular cancer in a subject at the time of onset of therapy. In subjects diagnosed as having the particular cancer, the efficacy of a method of the disclosure can be evaluated by measuring a lessening in the severity of the signs or symptoms in the subject or by the occurrence of a surrogate end-point for the disorder.

In some instances, such methods help identify an individual as having a predisposition for the development of the disease, and/or provide a means for detecting the disease prior to the appearance of actual clinical symptoms. A more definitive diagnosis of this type has the potential to allow health professionals to employ preventative measures or aggressive treatment earlier thereby preventing the development or further progression of the cancer.

In certain embodiments, expression of a gene product, a gene sequence, or a protein sequence in the epidermal sample is predictive of response to treatment if expression of the gene product, gene sequence, or protein sequence at the first time point is different in subjects that respond to treatment compared to subjects that do not respond to treatment. It will be understood that a variety of statistical analysis can be performed to identify a statistically significant association between expression of the gene product and response of the subject to the treatment. In some embodiments, the expression of the gene product, in certain examples, is elevated in subjects that will not respond to treatment. In some embodiments, the expression of the gene product, in certain examples, is decreased in subjects that will not respond to treatment. In some embodiments, the sequence of the gene product, in certain examples, is different in subjects that will not respond to treatment. In some embodiments, the sequence of a gene, in certain examples, is different in subjects that will not respond to treatment. Furthermore, expression of a target gene product, gene sequence, or protein sequence can predict a level of response to treatment, for example partial or temporary response to treatment versus a full response. In some embodiments, a target gene product is a nucleic acid molecule. In some embodiments, a target gene product is a polypeptide.

In some embodiments, provided herein is a non-invasive method for predicting response to treatment for melanoma, including applying an adhesive tape to the skin of a subject afflicted with melanoma in a manner sufficient to isolate an epidermal sample that includes a gene product. In some embodiments, a target gene product is detected in the epidermal sample, whose expression is indicative of a response to treatment, thereby predicting response to treatment for melanoma. In some embodiments, a target gene product is a nucleic acid molecule. In some embodiments, a target gene product is a polypeptide.

Certain embodiments provided herein, are based in part on the discovery that the expression and/or sequence of certain genes can be used to monitor response to therapy. Accordingly, in another embodiment, provided herein is a method for monitoring a response of a human subject to treatment for melanoma, including applying an adhesive tape to the skin of the subject being treated for melanoma at a first time point and at least a second time point, in a manner sufficient to isolate an epidermal sample adhering to the adhesive tape at the first time point and at the second time point. In some embodiments, the epidermal sample includes a gene product, wherein a change in expression of the gene product between the first time point and the second time point is indicative of a change in severity or level of melanoma. In some embodiments, a target gene product is a nucleic acid molecule. In some embodiments, a target gene product is a polypeptide. In some embodiments, the epidermal sample includes a gene, wherein a change in sequence of the gene between the first time point and the second time point is indicative of a change in severity or level of melanoma. In some embodiments, the epidermal sample includes a polypeptide, wherein a change in sequence of the polypeptide between the first time point and the second time point is indicative of a change in severity or level of melanoma.

In some embodiments, provided herein is a method for detecting a response of a subject to treatment for melanoma or monitoring the response of a subject to treatment for melanoma over a period of time, comprising: treating the subject for a skin disease or condition state; applying an adhesive tape to the skin of the subject in a manner sufficient to isolate an epidermal sample, wherein the epidermal sample includes a gene or gene expression product; and detecting a target gene product in the sample. Expression of the target gene product is informative regarding pathogenesis of melanoma. Therefore, the method identifies a response of the subject to treatment for melanoma. In some embodiments, a target gene product is a nucleic acid molecule. In some embodiments, a target gene product is a polypeptide. In some embodiments, the sequence of the target gene is informative regarding pathogenesis of melanoma. In some embodiments, the sequence of the polypeptide expression from the target gene is informative regarding pathogenesis of melanoma.

In some embodiments, the detection of the gene product is a qualitative detection of whether the target gene product is expressed. In some embodiments, the detection of the target gene product is quantitative assessment of the expression level of the target gene product. In some embodiments, the method is performed both prior to treatment and after treatment. In some embodiments, the method is performed after treatment, but before a change in severity or level of melanoma is observed. In some embodiments, the method is performed at multiple time points during treatment.

Time points for the monitoring and response-to-treatment methods provided herein, include any interval of time. In some embodiments, the time points are 1 day, 2 days, 3 days, 4 days, 5 days 6 days, 1 week, 2 weeks, 3, weeks, 4 weeks, 1 month, 2 months, 3 months, 4 months, 5 months, 6 months, 7 months, 8 months, 9 months, 10 months, 11 months, 1 year, 2 years or longer apart.

In some embodiments, skin samples are obtained at any number of time points, including 2, 3, 4, 5, 6, 7, 8, 9, 10, 11, 12, or more time points.

In some embodiments, comparison of expression analysis data from different time points is performed using any of the known statistical methods for comparing data points to assess differences in the data, including time-based statistical methods such as control charting. In some embodiments, the identity, severity or level of melanoma is identified in the time series, for example, by comparing expression levels to a cut-off value, or by comparing changes in expression levels to determine whether they exceed a cut-off change value, such as a percent change cut-off value. In certain aspects, the first time point is prior to treatment, for example, prior to administration of a therapeutic agent, and the second time point is after treatment. In some embodiments, the statistical methods are performed and analyzed using a software program or module performed on a computer processor. In some embodiments, the information regarding expression analysis data is an article and/or product of the methods described herein.

In some embodiments, the change in expression levels of at least one gene product is an increase or decrease in expression. Depending on the target gene product, an increase or decrease indicates a response to treatment, or a lack of response. For example, in some embodiments, the gene product is a nucleic acid comprising WFDC3, SLC16A6, DUSP4, PPAP2A, NDUFAF4, BCL2A1, DMXL2, SMG1, SLC2A3, VEGFA, BEX4, SH3BP5, RAB27A, FEM1A, CYTH3, IRGQ, EFNA5, NAMPT, PNPT1, or HNRNPH3 and a decrease or increase in expression at the second time point as compared to the first time point is indicative of positive response to treatment for melanoma. As another example, in some embodiments, the gene product detected is a polypeptide that is expressed by any of the following genes WFDC3, SLC16A6, DUSP4, PPAP2A, NDUFAF4, BCL2A1, DMXL2, SMG1, SLC2A3, VEGFA, BEX4, SH3BP5, RAB27A, FEM1A, CYTH3, IRGQ, EFNA5, NAMPT, PNPT1, and HNRNPH3, and a decrease or increase in expression at the second time point as compared to the first time point is indicative of positive response to treatment for melanoma. In some embodiments, the gene product is a nucleic acid that encodes a protein expressed by any of the following genes WFDC3, SLC16A6, DUSP4, PPAP2A, NDUFAF4, BCL2A1, DMXL2, SMG1, SLC2A3, VEGFA, BEX4, SH3BP5, RAB27A, FEM1A, CYTH3, IRGQ, EFNA5, NAMPT, PNPT1, and HNRNPH3, and an increase or decrease in expression at the second time point as compared to the first time point is indicative of positive response to treatment for melanoma. As another example, in some embodiments, the gene product detected is a polypeptide that is expressed by any of the following genes WFDC3, SLC16A6, DUSP4, PPAP2A, NDUFAF4, BCL2A1, DMXL2, SMG1, SLC2A3, VEGFA, BEX4, SH3BP5, RAB27A, FEM1A, CYTH3, IRGQ, EFNA5, NAMPT, PNPT1, and HNRNPH3, and an increase or decrease in expression at the second time point as compared to the first time point is indicative of positive response to treatment for melanoma.

In some embodiments, more than one target gene expression product is detected. In some embodiments, a population of target gene expression products are detected. In some embodiments, the method for detecting a population of target gene products is performed using a microarray.

In some embodiments where expression of more than one gene or gene expression product is analyzed, the detection is performed using a microarray. In some examples, the microarray includes an array of sequence specific nucleic acid probes. In some examples, the microarray includes an array of sequence specific polynucleotide probes. In some embodiments, the microarray includes an array of sequence specific nucleic acid probes directed to 2 or more, 3 or more, 4 or more, 5 or more, 6 or more, 7 or more, 8 or more, 9 or more, 10 or more, or all of the following genes WFDC3, SLC16A6, DUSP4, PPAP2A, NDUFAF4, BCL2A1, DMXL2, SMG1, SLC2A3, VEGFA, BEX4, SH3BP5, RAB27A, FEM1A, CYTH3, IRGQ, EFNA5, NAMPT, PNPT1, and HNRNPH3. In some embodiments, the microarray includes an array of sequence specific polynucleotide probes directed to 2 or more, 3 or more, 4 or more, 5 or more, 6 or more, 7 or more, 8 or more, 9 or more, 10 or more, or all of the polypeptides expressed from the following genes WFDC3, SLC16A6, DUSP4, PPAP2A, NDUFAF4, BCL2A1, DMXL2, SMG1, SLC2A3, VEGFA, BEX4, SH3BP5, RAB27A, FEM1A, CYTH3, IRGQ, EFNA5, NAMPT, PNPT1, and HNRNPH3.

In some embodiments, the relative amount of the gene product is increased in an epidermal skin sample from a melanoma lesion or an epidermal skin sample from a suspected melanoma lesion compared to a control by about 2-fold, 3-fold, 4-fold, 5-fold, 10-fold, 20-fold, 30-fold, 40-fold, 50-fold, 60-fold, 70-fold, 80-fold, 90-fold, or 100-fold. In some embodiments, the relative amount of the gene product is decreased in an epidermal skin sample from a melanoma lesion or an epidermal skin sample from a suspected melanoma lesion compared to a control by about 2-fold, 3-fold, 4-fold, 5-fold, 10-fold, 20-fold, 30-fold, 40-fold, 50-fold, 60-fold, 70-fold, 80-fold, 90-fold, or 100-fold. In some embodiments, the control is a normal skin sample. In some embodiments, the control is a value obtained from a database of relative expression values. In some embodiments, the control is a value obtained from a known relative expression values.

In some embodiments, expression of a target gene believed to be involved in melanoma is detected in a pigmented skin lesion using a tape stripping method provided herein. In some embodiments, if expression, elevated expression, or decreased expression is detected, a treatment is administered to the subject that blocks a function of the target gene. In some embodiments, a target gene believed to be involved in melanoma is detected in a pigmented skin lesion using a tape stripping method provided herein. In some embodiments, if a sequence mutation of differential sequence is detected, a treatment is administered to the subject that blocks a function of the target gene. Accordingly, in some embodiments, the methods provided herein are used to determine whether the subject is likely to respond to treatment with a biologic that targets a particular gene that exhibits differential expression in a pigmented skin lesion.

### Pigmented Skin Lesion Sampling Methods

Samples from a tissue can be isolated by any number of means well known in the art. Invasive methods for isolating a sample include, but are not limited to the use of needles or scalpels, for example during biopsies of various tissues. Non-invasive methods for isolating a sample include, but are not limited to tape-stripping and skin scraping.

In certain embodiments, the method of detecting genes or gene expression products in the skin involves applying an adhesive tape to a target area of the skin in a manner sufficient to isolate an epidermal sample adhering to the adhesive tape, wherein the epidermal sample comprises cellular material comprising a gene or gene expression product. The gene or gene products in the epidermal sample are then detected. In some embodiments, gene products are applied to a microarray to detect the gene products. In some embodiments, the gene product is isolated from the epidermal sample. In some embodiments, the gene product is a nucleic acid molecule, such as a RNA or a DNA molecule. In some embodiments, the nucleic acid is amplified. In some embodiments, the gene product is a polypeptide. In some embodiments, the gene is sequenced. In some embodiments, the polypeptide is sequenced.

Accordingly, in one embodiment, the present disclosure employs a non-invasive tape stripping technology to obtain samples of suspicious lesions. As such, in exemplary embodiments, DNA microarray assays are used to create a non-invasive diagnostic for melanoma and/or distinguishing melanoma from solar lentigo. Tape-stripping removes superficial cells from the surface of the skin as well as adnexal cells. Small amounts of nucleic acid molecules isolated and/or purified from tape-stripped cells can be amplified and used for microarray analyses and quantitative PCR. In addition, proteins obtained from the lysed cells may be quantitated for diagnosis of disease. An exemplary diagnosis includes distinguishing melanoma in situ from invasive melanoma. Consequently, tape-stripping is a non-invasive diagnostic method, which does not interfere with subsequent histological analyses, thereby bypassing a major limitation to current expression profiling studies on melanoma. While tape stripping will primarily sample superficial cells from the epidermis, this method holds great promise in the diagnoses and prognosis prediction in pigmented lesions for the following reasons: First, in contrast to benign nevi, in many melanomas the pigmented cells migrate into the epidermis and/or adnexa. Consequently, this feature, in some instances, is helpful to differentiate benign pigmented lesions from melanomas based on tape stripping. Second, there are changes in the dermis and epidermis adjacent to melanoma. The epidermal hyperplasia overlying melanoma seems to correlate with both angiogenesis and metastatic potential; these changes are expected to be sampled with the tape stripping method. Finally, some advanced melanomas do reach the surface of the skin and melanoma cancer cells would be sampled directly by the tape stripping. In addition tape stripping is useful in the care of patients with multiple pigmented lesions where it is unpractical to biopsy each and every lesion. Accordingly, the present disclosure demonstrates that stratum corneum RNA, harvested by tape stripping with Epidermal Genetic Information Retrieval (EGIR) (see U.S. Pat. No. 6,949,338, incorporated herein by reference), can be used to distinguish melanoma from dysplastic nevi in suspicious pigmented lesions.

As indicated, the tape stripping methods provided herein typically involve applying an adhesive tape to the skin of a subject and removing the adhesive tape from the skin of the subject one or more times. In certain examples, the adhesive tape is applied to the skin and removed from the skin about one to ten times. Alternatively, about one to about ten adhesive tapes can be sequentially applied to the skin and removed from the skin. In some instances, these adhesive tapes are then be combined for further analysis. Accordingly, an adhesive tape can be applied to and removed from a target site 10, 9, 8, 7, 6, 5, 4, 3, 2, or 1 time, and/or 10, 9, 8, 7, 6, 5, 4, 3, 2, or 1 adhesive tape can be applied to and removed from the target site, for example, a pigmented skin lesion. In one illustrative example, the adhesive tape is applied to the skin between about one and eight times, in another example, between one and five times, and in another illustrative example the tape is applied and removed from the skin four times.

In some embodiments, the adhesive tape is pliable. In some embodiments, the adhesive tape comprises a non-polar polymer adhesive. In some embodiments, the adhesive tape comprises a rubber-based adhesive. In some embodiments, the adhesive tape does not comprise latex, silicone, or latex and silicone.

In certain instances, non-polar, pliable adhesive tapes, including plastic-based adhesive tapes, are effective for obtaining epidermal samples from the skin. In certain instances, non-polar, pliable adhesive tapes, including plastic-based adhesive tapes, are more effective for obtaining epidermal samples from the skin than other types of adhesive tapes. Accordingly, in some embodiments, a non-polar, pliable adhesive tapes are applied in as few as 10 or less tape strippings, such as 9, 8, 7, 6, 5, 4, 3, 2, or 1 tape stripping, to obtain a sample. In some embodiments, the tape strippings method is employed to isolate a gene or gene expression product from the epidermis of skin for gene expression analysis, gene sequence analysis, protein expression analysis, or protein sequence analysis.

In some embodiments, the rubber based adhesive is a synthetic rubber-based adhesive. In some embodiments, the rubber based adhesive has high peel, high shear, and high tack. For example, in some embodiments, the rubber based adhesive has a peak force tack that is at least 25%, 50%, or 100% greater than the peak force tack of an acrylic-based tape such as D-squame™. D-squame.™ has been found to have a peak force of 2 Newtons. In some embodiments, the peak force of the rubber based adhesive used for methods provided herein is about 4 Newtons or greater. In some embodiments, the rubber based adhesive has adhesion that is greater than 2 times, 5 times, or 10 times that of acrylic based tape. D-squame™ has been found to have adhesion of 0.0006 Newton meters. In some embodiments, the rubber based tape provided herein has an adhesion of about 0.01 Newton meters using a texture analyzer. In some embodiments, the adhesive used in the methods provided herein has higher peel, shear and tack compared to other rubber adhesives, such as those used for medical application and Duct tape.

In some embodiments, the rubber-based adhesive is more hydrophobic than acrylic adhesives. In some embodiments, the rubber based adhesive is inert to biomolecules and to chemicals used to isolate biomolecules, including proteins and nucleic acids, such as DNA and RNA. In some embodiments, the rubber-based adhesive is relatively soft compared to other tapes such as D-squame™.

In some embodiments, the rubber-based adhesive is on a support, such as a film, that makes the tape pliable and flexible. In certain aspects, the tape is soft and pliable. As used herein, “pliable” tape is tape that is easily bent or shaped. As used herein, “soft and pliable” tape is tape that is easily bent or shaped and yields readily to pressure or weight. In some embodiments, the film is made of any of many possible polymers, provided that the tape is pliable and can be used with a rubber adhesive. In some embodiments, the film is a polyurethane film such as skin harvesting tape (Product No. 90068) available from Adhesives Research, Inc (Glen Rock, Pa.). In some embodiments, the thickness is varied provided that the tape remains pliable. For example, in some embodiments, the tape is about 0.5 mm to about 10 mm in thickness, such as about 1.0 to about 5.0 mm in thickness. In one example, the tape contains a rubber adhesive on a 3.0 mm polyurethane film.

Virtually any size and/or shape of adhesive tape and target skin site size and shape can be used and analyzed, respectively, by the methods of the present disclosure. For example, adhesive tape can be fabricated into circular discs of diameter between 10 millimeters and 100 millimeters, for example between 15 and 25 millimeters in diameter. The adhesive tape can have a surface area of between about 50 mm² and 1000 mm², between about 100 mm² to 500 mm² or about 250 mm².

Described herein, in certain embodiments, are adhesive tapes comprising an epidermal sample of a suspected melanoma lesion that comprises a gene or gene product expressed by 1, 2, 3, 4, 5, 6, 7, 8, 9, 10, 11, 12, 13, 14, 15, 16, 17, 18, 19, or 20 genes in any of WFDC3, SLC16A6, DUSP4, PPAP2A, NDUFAF4, BCL2A1, DMXL2, SMG1, SLC2A3, VEGFA, BEX4, SH3BP5, RAB27A, FEM1A, CYTH3, IRGQ, EFNA5, NAMPT, PNPT1, or HNRNPH3, wherein the epidermal sample is of a sufficient quantity to allow determination of the relative amount of gene product present in the epidermal sample, the sequence of the gene product in the sample, or the sequence of the protein in the sample. Described herein, in certain embodiments, are adhesive tapes comprising an epidermal sample of a suspected melanoma lesion that comprises a gene or gene product expressed by 1, 2, 3, 4, 5, or 6 genes in any of WFDC3, SLC16A6, DUSP4, PPAP2A, NDUFAF4, or BCL2A1, wherein the epidermal sample is of a sufficient quantity to allow determination of the relative amount of gene product present in the epidermal sample, the sequence of the gene product in the sample, or the sequence of the protein in the sample.

In another embodiment, the sample is obtained by means of an invasive procedure, such as biopsy. Biopsies may be taken instead of or after tape stripping and are subjected to standard histopathologic analysis. Analysis of biopsy samples taken simultaneously with tape stripping samples may then be correlated with the data generated from one or more of analysis of selected lesion RNA samples by DNA microarray, correlation of gene expression data with histopathology, and creation of a candidate expression molecular signature for diagnosis of melanoma. In some embodiments, the analysis methods are performed and analyzed using a software program or module performed on a computer processor.

As used herein, “biopsy” refers to the removal of cells or tissues for analysis. There are many different types of biopsy procedures known in the art. The most common types include: (1) incisional biopsy, in which only a sample of tissue is removed; (2) excisional biopsy, in which an entire lump or suspicious area is removed; and (3) needle biopsy, in which a sample of tissue or fluid is removed with a needle. When a wide needle is used, the procedure is called a core biopsy. When a thin needle is used, the procedure is called a fine-needle aspiration biopsy. Other types of biopsy procedures include, but are not limited to, shave biopsy, punch biopsy, curettage biopsy, and in situ biopsy. In another embodiment, the skin sample is obtained by scraping the skin with an instrument to remove one or more genes or gene expression products from the skin.

The skin sample obtained using the tape stripping method includes, epidermal cells including cells comprising adnexal structures. In certain illustrative examples, the sample includes predominantly epidermal cells, or even exclusively epidermal cells. The epidermis consists predominantly of keratinocytes (>90%), which differentiate from the basal layer, moving outward through various layers having decreasing levels of cellular organization, to become the cornified cells of the stratum corneum layer. Renewal of the epidermis occurs every 20-30 days in uninvolved skin. Other cell types present in the epidermis include melanocytes, Langerhans cells, and Merkel cells. As illustrated in the Examples herein, the tape stripping method of the present disclosure is particularly effective at isolating epidermal samples.

In another aspect, the methods of the present disclosure involve in situ analysis of the pigmented skin lesion for characterization thereof. For in situ methods, genes or gene expression products do not need to be isolated or purified from the subject prior to analysis. In one embodiment, detectably labeled probes are contacted with a cell or tissue of a subject for visual detection of expressed RNA to characterize the pigmented skin lesion.

In some embodiments, the methods of the present disclosure can be used with less than 100,000 skin cells. In some embodiments, the methods of the present disclosure are practiced with less than 75,000 skin cells. In some embodiments, the methods of the present disclosure are practiced with less than 50,000 skin cells. In some embodiments, the methods of the present disclosure are practiced with less than 25,000 skin cells. In some embodiments, the methods of the present disclosure are practiced with less than 10,000 skin cells. In some embodiments, the methods of the present disclosure are practiced with less than 5,000 skin cells. In some embodiments, the methods of the present disclosure are practiced with less than 1,000 skin cells. In some embodiments, the methods of the present disclosure are practiced with less than 900 skin cells. In some embodiments, the methods of the present disclosure are practiced with less than 800 skin cells. In some embodiments, the methods of the present disclosure are practiced with less than 700 skin cells. In some embodiments, the methods of the present disclosure are practiced with less than 600 skin cells. In some embodiments, the methods of the present disclosure are practiced with less than 500 skin cells. In some embodiments, the methods of the present disclosure are practiced with less than 400 skin cells. In some embodiments, the methods of the present disclosure are practiced with less than 300 skin cells. In some embodiments, the methods of the present disclosure are practiced with less than 200 skin cells. In some embodiments, the methods of the present disclosure are practiced with less than 100 skin cells.

The target gene or gene expression product that is ultimately analyzed can be prepared synthetically (in the case of control sequences), but typically is purified from the skin sample and subjected to one or more preparative steps. The nucleic acid, in some instances, is purified to remove or diminish one or more undesired components from the biological sample or to concentrate it. Conversely, where the nucleic acid is too concentrated for the particular assay, it may be diluted.

### Methods for Isolation of a Gene or Gene Expression Product

In certain embodiments, the gene or gene expression products are isolated from epidermal samples. In some embodiments, the cells of the epidermal samples are lysed. In some embodiments, the cells of the epidermal samples are lysed and the genes or gene expression products are isolated and/or purified from lysed cells.

In certain embodiments, nucleic acid molecules are isolated and/or purified from the lysed cells and cellular material by any number of means well known to those skilled in the art. For example, in some embodiments, any of a number of commercial products available for isolating nucleic acid molecules, including, but not limited to, RNeasy™ (Qiagen, Valencia, Calif.) and TriReagent™ (Molecular Research Center, Inc, Cincinnati, Ohio), is used. In some embodiments, the isolated and/or nucleic acid molecules are then tested or assayed for particular nucleic acid sequences. In some embodiments, the isolated and/or purified nucleic acid molecules are then tested or assayed for a nucleic acid sequence that represents a gene or gene expression product of any of the genes WFDC3, SLC16A6, DUSP4, PPAP2A, NDUFAF4, BCL2A1, DMXL2, SMG1, SLC2A3, VEGFA, BEX4, SH3BP5, RAB27A, FEM1A, CYTH3, IRGQ, EFNA5, NAMPT, PNPT1, or HNRNPH3. Methods of detecting a target nucleic acid molecule within a nucleic acid sample are well known in the art. In some embodiments, detecting a target nucleic acid molecule involves a hybridization technique such as a microarray analysis or sequence specific nucleic acid amplification. In some embodiments, detecting a target nucleic acid molecule involves sequencing.

In some embodiments, one or more of the nucleic acid molecules in a sample provided herein, such as an epidermal sample, is amplified before or after they are isolated, purified and/or detected. The term “amplified” refers to the process of making multiple copies of the nucleic acid from a single nucleic acid molecule. In some embodiments, the amplification of nucleic acid molecules is carried out in vitro by biochemical processes known to those of skill in the art. In some embodiments, the amplification agent is any compound or system that will function to accomplish the synthesis of primer extension products, including enzymes. It will be recognized that various amplification methodologies can be utilized to increase the copy number of a target nucleic acid in the nucleic acid samples obtained using the methods provided herein, before and after detection. Suitable enzymes for this purpose include, for example, E. coli DNA polymerase I, Taq polymerase, Klenow fragment of E. coli DNA polymerase I, T4 DNA polymerase, other available DNA polymerases, T4 or T7 RNA polymerase, polymerase muteins, reverse transcriptase, ligase, and other enzymes, including heat-stable enzymes (i.e., those enzymes that perform primer extension after being subjected to temperatures sufficiently elevated to cause denaturation or those using an RNA polymerase promoter to make a RNA from a DNA template, i.e. linearly amplified aRNA).

Suitable enzymes will facilitate incorporation of nucleotides in the proper manner to form the primer extension products that are complementary to each nucleotide strand. Generally, the synthesis will be initiated at the 3′-end of each primer and proceed in the 5′-direction along the template strand, until synthesis terminates, producing molecules of different lengths. There can be amplification agents, however, that initiate synthesis at the 5′-end and proceed in the other direction, using the same process as described above. In any event, the method provided herein is not to be limited to the amplification methods described herein since it will be understood that virtually any amplification method can be used.

In some embodiments, polymerase chain reaction (PCR) is employed for nucleic acid amplification (described, e.g., in U.S. Pat. Nos. 4,683,202 and 4,683,195). It will be understood that optimal conditions for a PCR reaction can be identified using known techniques. In one illustrative example, RNA is amplified using the MessageAmp™aRNA kit (as disclosed in the Examples herein). In various implementations, PCR is real-time quantitative PCR.

In some embodiments, the primers for use in amplifying the polynucleotides of the disclosure are prepared using any suitable method, such as conventional phosphotriester and phosphodiester methods or automated embodiments thereof so long as the primers are capable of hybridizing to the polynucleotides of interest. One method for synthesizing oligonucleotides on a modified solid support is described in U.S. Pat. No. 4,458,066. The exact length of primer will depend on many factors, including temperature, buffer, and nucleotide composition. The primer must prime the synthesis of extension products in the presence of the inducing agent for amplification.

Primers used according to the method of the disclosure are complementary to each strand of nucleotide sequence to be amplified. The term “complementary” means that the primers must hybridize with their respective strands under conditions, which allow the agent for polymerization to function. In other words, the primers that are complementary to the flanking sequences hybridize with the flanking sequences and permit amplification of the nucleotide sequence. The 3′ terminus of the primer that is extended can have perfect base paired complementarity with the complementary flanking strand, or can hybridize to the flanking sequences under high stringency conditions.

In some embodiments, upon isolation and optional amplification, expression of one or more genes is analyzed. Analyzing expression includes any qualitative or quantitative method for detecting expression of a gene, many of which are known in the art. The methods of analyzing expression of the present disclosure can utilize a biochip, or other miniature high-throughput technology, for detecting expression of two or more genes.

In some embodiments, the methods provided involve isolation of RNA, including messenger RNA (mRNA), from a skin sample. In some embodiments, RNA is single stranded or double stranded. In some embodiments, enzymes and conditions optimal for reverse transcribing the template to DNA well known in the art are used. In some embodiments, the RNA is amplified to form amplified RNA. In some embodiments, the RNA is subjected to RNAse protection assays. In some embodiments, a DNA-RNA hybrid that contains one strand of each is used. In some embodiments, a mixture of polynucleotides is employed, or the polynucleotides produced in a previous amplification reaction, using the same or different primers are used. In certain examples, a nucleic acid to be analyzed is amplified after it is isolated and/or purified. It is not necessary that the sequence to be amplified be present initially in a pure form; it may be a minor fraction of a complex mixture.

### Kits

In another embodiment, provided herein are kits that include one or more reagents or devices for the performance of the methods disclosed herein. In some embodiments, provided is a kit for isolation, detection, and or analysis of a gene or gene expression product from an epidermal sample, such as a pigmented skin lesion.

In some embodiments, the kit includes an adhesive tape for performing methods provided herein. In some embodiments, the kit includes an adhesive tape for tape stripping skin, such as rubber-based, pliable adhesive tape. Accordingly, in some embodiments, provided herein is a kit, including a pliable adhesive tape made up at least in part, of a non-polar polymer. In certain aspects, the tape includes a rubber adhesive. In an illustrative example, the tape can be skin harvesting tape available (Product No. 90068) from Adhesives Research, Inc (Glen Rock, Pa.). In some embodiments, the kit includes instructions for performing tape strippings or for analyzing gene expression.

In some embodiments, the kit includes nucleic acid or polypeptide isolation reagents.

In some embodiments, the kit includes one or more detection reagents, for example probes and/or primers for amplification of, or hybridization to, a target nucleic acid sequence whose expression is related to melanoma. In some embodiments, the kit includes primers and probes for control genes, such as housekeeping genes. In some embodiments, the primers and probes for control genes are used, for example, in ΔCₜ calculations. In some embodiments, the probes or primers are labeled with an enzymatic, florescent, or radionuclide label. In some embodiments, the probe binds to a target nucleic acid molecule encoding a protein. In some embodiments, the probe is an antibody or ligand that binds the encoded protein. In some embodiments, probes are spotted on a microarray. In some embodiments, the microarray is provided in the kit.

The term “detectably labeled deoxyribonucleotide” refers to a deoxyribonucleotide that is associated with a detectable label for detecting the deoxyribonucleotide. For example, the detectable label is a radiolabeled nucleotide or a small molecule covalently bound to the nucleotide where the small molecule is recognized by a well-characterized large molecule. Examples of these small molecules are biotin, which is bound by avidin, and thyroxin, which is bound by anti-thyroxin antibody. Other labels are known to those of ordinary skill in the art, including enzymatic, fluorescent compounds, chemiluminescent compounds, phosphorescent compounds, and bioluminescent compounds.

In some embodiments, the kit includes one or more primer pairs, including a forward primer that selectively binds upstream of a gene whose expression is associated with psoriasis or irritant dermatitis, for example, on one strand, and a reverse primer, that selectively binds upstream of a gene involved in psoriasis or irritant dermatitis on a complementary strand. Primer pairs according to this aspect of the disclosure are typically useful for amplifying a polynucleotide that corresponds to a skin marker gene associated with melanoma using amplification methods described herein.

In some embodiments, a kit provided herein includes a carrier means being compartmentalized to receive in close confinement one or more containers such as vials, tubes, and the like, each of the containers comprising one of the separate elements to be used in a method provided herein. In some embodiments, a second container includes, for example, a lysis buffer. In some embodiments, the kit includes a computer-type chip on which the lysis of the cell will be achieved by means of an electric current.

In another embodiment, a kit of the disclosure includes a probe that binds to a portion of a gene of gene expression product of the following genes WFDC3, SLC16A6, DUSP4, PPAP2A, NDUFAF4, BCL2A1, DMXL2, SMG1, SLC2A3, VEGFA, BEX4, SH3BP5, RAB27A, FEM1A, CYTH3, IRGQ, EFNA5, NAMPT, PNPT1, or HNRNPH3. In another embodiment, the kit further includes a microarray that contains at least a fragment of a gene or a nucleic acid molecule or a protein product of any one of the following genes WFDC3, SLC16A6, DUSP4, PPAP2A, NDUFAF4, BCL2A1, DMXL2, SMG1, SLC2A3, VEGFA, BEX4, SH3BP5, RAB27A, FEM1A, CYTH3, IRGQ, EFNA5, NAMPT, PNPT1, or HNRNPH3. In some embodiments, many reagents are provided in a kit of the disclosure, only some of which should be used together in a particular reaction or procedure. For example, multiple primers are provided, only two of which are needed for a particular application.

The following examples are provided to further illustrate the advantages and features of the present disclosure, but are not intended to limit the scope of the disclosure. While they are typical of those that might be used, other procedures, methodologies, or techniques known to those skilled in the art may alternatively be used.

### EXAMPLES

### Example 1

### Tape Stripping to Recover Cellular Material from Pigmented Lesions

The following procedure is used to recover cellular material including cells comprising genes and gene expression products from pigmented lesions having melanoma from a subject. In contrast to normal skin, lesional skin has a preoperative biopsy diameter of greater than or equal to about 6 mm, but less than that of an adhesive tape. Multiple lesions are at least about 4 mm apart. The area of tape that touches the lesion is generously demarcated on the tape with an insoluble ink pen so that this area may be cut away from the surrounding tape as part of the cellular material extraction procedure.

Tapes are handled with gloved hands at all times. The site of the pigmented lesion is shaved, if necessary, to remove non-vellus hairs. The site is then cleansed with an alcohol wipe (70% isopropyl alcohol). The site is then air-dried completely before application of the tape. It is recommended to air-dry for approximately 2 minutes to ensure the site is completely dry before application of the tape.

The tape is applied to the pigmented lesion on the skin surface. If more than one tape is used, tapes are applied in sequential order. Pressure is applied directly over the lesion on the tape. The skin is held taut to ensure that the tape does not move while applying pressure. Using a surgical skin marker and/or a water soluble marker, a zone around the lesion is demarcated on the outer surface of the tape (opposite the site comprising a skin facing, adhesive matrix) such that the area of the lesion is encompassed within the inked boundary. The inked boundary may include about 1 mm around the lesion border.

The tape is slowly removed in one direction. The adhesive matrix of the tape now contains cellular material from the surface of the pigmented lesion. The tape is placed on a collection panel with the adhesive matrix comprising the skin cells facing down to protect the skin sample. A second, third, and fourth strip are sequentially applied to and removed from the same pigmented lesion and stored on the same collection panel. The strips are placed in a storage container and immediately placed on dry ice or at −20° C. or below until analysis.

### Example 2

### RNA Quantitation and Profiling

The study was divided into two separate phases, a sample collection and characterization phase (Phase 1) and an RNA profiling phase (Phase 2). In phase 1 the tape stripped samples and biopsied sample collections were performed by the principal investigator or trained individuals delegated by the principal investigator to obtain samples at various sites. All biopsies were subject to standard histopathologic analysis. The RNA profiling phase (Phase 2), includes, but is not limited to RNA purification and hybridization to DNA microarrays for gene expression profiling.

Materials and Reagents.

Adhesive tape was purchased from Adhesives Research (Glen Rock, Pa.) in bulk rolls. These rolls were custom fabricated into small circular discs, 17 millimeters in diameter, by Diagnostic Laminations Engineering (Oceanside, Calif.). Human spleen total RNA was purchased from Ambion (catalogue #7970; Austin, Tex.). RNeasy RNA extraction kit was purchased from Qiagen (Valencia, Calif.). Reverse transcriptase, PCR primers and probes, and TaqMan Universal Master Mix, which included all buffers and enzymes necessary for the amplification and fluorescent detection of specific cDNAs, were purchased from Applied Biosystems (Foster City, Calif.). MELT total nucleic acid isolation system was purchased from Ambion (Austin, Tex.).

RNA Isolation.

RNA was extracted from tapes using either pressure cycling technology (PCT; Garrett, Tao et al. 2002; Schumacher, Manak et al. 2002) or MELT total nucleic acid system. Tapes were extracted in pairs by insertion into a PULSE™ tube (Pressure Biosciences, Gaithersburg, Md.) with 1.2 mls of buffer RLT (supplied in the Qiagen RNeasy kit). PULSE™ tubes were inserted into the PCT-NEP2017 pressure cycler and the sample was extracted using the following parameters: room temperature; 5 pressure cycles of 35 Kpsi with pressure held for 20 seconds at the top and bottom of each cycle. After pressure extraction the buffer was removed and used to process the remaining tapes used to strip that site; the buffer was then processed according to the standard Qiagen RNeasy protocol for the collection of larger RNAs (>200 nucleotides) by application to a purification column to which large RNA molecules (i.e. mRNAs) bind, while the column flow-through is saved for microRNA purification. The column flow-through, which contains miRNA separated from mRNA, is processed according to the Qiagen miRNA purification procedure to purify the microRNA. RNA from the 2 sites stripped on each subject was pooled to create a single sample from each subject.

RNA Isolation Using MELT Total Nucleic Acid Protocol.

Tapes were extracted in a 2 ml Eppendorf tube with 192 ml MELT buffer plus 8 ml of MELT cocktail and vortexed for 10 minutes at room temperature. The MELT lysates were transferred to the dispensed binding bead master mix after spinning down for 3 minutes at >10,000*g and washed with 300 ml of Wash Solution 1 and 2. RNAs were eluted in 100 ml of elution solution.

Quantitation of mRNA.

Experimental data is reported as the number of PCR cycles required to achieve a threshold fluorescence for a specific cDNA and is described as the “Ct” value (Gibson, Heid et al. 1996; Heid, Stevens et al. 1996; AppliedBiosystems 2001). Quantitation of total RNA mass was performed as previously described (Wong, Tran et al. 2004). Briefly, RNA mass recovered from tapes is determined by using real-time quantitative PCR with reference to a standard curve (Ct, actin vs. log [RNA]; AppliedBiosystems 2001) created from commercially purchased human spleen total RNA. The average of replicate Ct, actin values was used to calculate the concentration of RNA in a sample with reference to the standard curve.

RNA Amplification and Array Hybridization.

RNA was isolated by the Multi-Enzymatic Liquefaction of Tissue method (Ambion, Austin, Tex.) and amplified using the WT-Ovation pico amplification system (NuGen, San Carlos, Calif.). The amplified RNA was hybridized to Affymetrix U133 plus 2.0 microarray and data were processed and analyzed using R from Bioconductor.

Preprocessing GeneChip Data.

The image files from scanning the Affymetrix GeneChips with the Affymetrix series 3000 scanner were converted using GCOS software (Affymetrix) to “CEL” format files. Normalization of CEL files was carried out using software from the Bioconductor suite. In particular, a robust multiarray analysis with adjustments for optical noise and binding affinities of oligonucleotide probes (Wu et al., 2006; and Wu et al., 2004) as implemented by the function “just.gcrma” in the “gcrma” package was used to normalize the GeneChip Data.

Statistical Approach for Microarray Data Analysis.

Two generic statistical problems are addressed: (i) identifying genes that are have gene differential expression for different characteristics of melanoma (i.e. melanoma in situ versus invasive melanoma) and (ii) forming (and evaluating) rules for classification of melanoma lesions into groups based on gene expression data.

The most important grouping divides melanoma in situ from invasive melanoma on the basis of biopsy results. For identifying differentially expressed genes, permutation based estimates of false discovery rates are preferred. Scripts for the R quantitative programming environment were developed to implement these methods previously, however, the “siggenes” package from the Bioconductor suite was used in this project. The development of classification rules rely on resampling methods (k-fold cross-validation, the 632 plus bootstrap, and/or bagging applied to the naive Bayes molecular signature and the nearest shrunken centroid molecular signature and the support vector machine (SVM). The implementation used is k-fold cross-validation. Within each of the k train/test cycles an initial screen of the training data for differentially expressed genes was performed and genes were ordered according to their posterior probability of differential expression. Naive Bayes and nearest shrunken centroid molecular signatures based on the r genes with the highest posterior probability of differential expression were formed choosing enough values of r between 1 and 1024 to allow accurate interpolation of the classification error rate. The “one se rule” was applied to the error rates for the test sets to choose the molecular signature that minimizes the error rate. For SVM, an internal 632+ bootstrap was applied to each training sample to select the number of genes used in forming the molecular signature. The “1 se rule” error rates from the k test sets were used to characterize the classification accuracy.

QC Metrics for RNA, Amplified cDNA and Microarray Data.

Following informed consent, the pigmented lesion was tape stripped using EGIR and then biopsied as per standard of care. The resulting RNA isolated from the EGIR tape was amplified and profiled on the Affymetrix U133 plus 2.0 GeneChip. Microarray data were normalized by the GCRMA algorithm. To assure high quality of microarray data are generated, QC metrics were established for RNA, amplified cDNA and microarray data. The quality of RNA was assessed by capillary electrophoresis using the Experion system (Biorad, Hercule, Calif.) and RNA with at least one visible 18S rRNA was further processed for RNA amplification. The amplified cDNA was quantified by the Nanodrop system and quality of the amplified cDNA was also assessed by the Experion system. Quality of the array data was further assessed using simpleaffy program in R and the array data with scaling factor less than 5.0 and % present call greater than 30% were used for further data analysis.

Class Modeling-PAM.

After passing the array data QC, melanoma in situ, and invasive melanoma skin samples were further analyzed. First, certain gene expression values across all samples were filtered out and probesets were tested. The probesets were subjected to a statistical analysis for differentially expressed genes among melanomas using ANOVA (p<0.05), multiple testing correction algorithm (Westafall and Young permutation) and false discover rate (FDR) of 0.05. Of the original genes, a 20 gene panel (Table 1) was found to be a potential melanoma molecular signature. Further testing identified a 6-gene molecular signature (WFDC3, SLC16A6, DUSP4, PPAP2A, NDUFAF4 and BCL2A1) which is useful to discriminate melanoma in situ from invasive melanoma. The genes and respective molecular signature panels were analyzed using the Prediction Analysis of Microarrays (PAM) software freely available from Stanford University (Stanford, Calif.).

The PAM software uses a modification of the nearest centroid method, which computes a standardized centroid for each class in a training set. This refers to the average gene expression for each gene in each class divided by the within-class standard deviation for that gene. Nearest centroid classification takes the gene expression profile of a new sample, and compares it to each of these class centroids. The class, whose centroid it is closest to, in squared distance, is the predicted class for that new sample.

These genes were all subjected to a hierarchical clustering analysis and the melanoma specimens grouped together and were clearly distinguished from melanoma in situ and invasive melanoma. These data suggest stratum corneum RNA, harvested by tape stripping with EGIR (epidermal genetic information retrieval), can be used to distinguish melanoma between melanoma in situ and invasive melanoma in suspiciously pigmented lesions.

### Example 3

### Assessment of Performance of Multi-Gene Biomarker for Melanoma Characterization

Custom TaqMan OpenArray (OA) qPCR Plate.

The OpenArray real-time quantitative PCR system (Life Technologies, Carlsbad, Calif.) was chosen for investigation.

Latin Square Experimental Design and Method.

20 cloned human genes were used, including: WFDC3, SLC16A6, DUSP4, PPAP2A, NDUFAF4, BCL2A1, DMXL2, SMG1, SLC2A3, VEGFA, BEX4, SH3BP5, RAB27A, FEM1A, CYTH3, IRGQ, EFNA5, NAMPT, PNPT1, and HNRNPH. Each of the 20 genes were pooled into groups and diluted to copies of 0, 64, 256, 1024, 4096, 1.6E+04, 6.6E+04, 2.6E+05, 1.0E+06, 4.2E+06, 1.7E+07, 6.7E+07 and 2.7E+08.

For each qPCR experiment, 20 groups of genes in 20 different copies were assayed in triplicate in the presence of a complex human background, produced from EGIR-harvested normal human skin that was amplified with 20 primer sets for non-target mRNAs, and the ACTB internal control gene. After assaying on the OA platform, the qPCR data was de-convoluted to ascertain sensitivity and linear dynamic range for each of the 20 genes.

Gene Expression Profiling of EGIR Specimens.

Specimens were collected at clinical sites and shipped and stored at −80° C. Total RNA was extracted using MELT (Ambion, Inc.). Part of the tape demarcated over each pigmented lesion was used for analysis, and RNA product was pooled from 4 tapes per lesion.

For microarray analysis, RNA was amplified using the WT-Ovation FFPE kit (NuGen, Inc.). The microarray assay used was the U133 plus 2.0 GeneChip (Affymetrix, Inc.) Data was quality checked with Simpleaffy and normalized with the GCRMA algorithm (Bioconductor).

For real-time quantitative PCR assays, RNA was pre-amplified with 14 cycles of PCR using a pool of TaqMan primer/probe-sets (ABI, Inc). The real-time quantitative PCR data was normalized with 3 internal controls using geometric mean.

Class Prediction Modeling.

TreeNet® (Salford Systems, Inc.) was used to generate a prediction model from the training dataset established for each platform (i.e. OA and microarray) for the gene targets selected from the discovery research. An independent test dataset was used to validate the class prediction model (i.e., classifier) established from the training dataset.

Clinical Protocol.

Inclusion criteria: Subjects are 18 or older and have a pigmented skin lesion that is suspicious for melanoma and requires biopsy. Lesion size is 4 mm or greater, and if multiple lesions are present, there must be >4 mm of separation between the lesions.

Exclusion criteria: Exclusion criteria include; the presence of a lesion that is ulcerated, bleeding or weeping; use of topical moisturizer or sunscreen on lesion sites within 24 h; and/or allergy to tape or latex.

Procedures.

Lesions were tape stripped using 4 adhesive tapes in a kit. The lesion edge was demarcated on the tape, if the lesion was smaller than 17 mm in diameter. Lesions were biopsied as per standard of care. Biopsy specimens were reviewed by primary and central dermatopathology.

Purpose 1: Ascertain adequacy of EGIR-harvested normal skin RNA, pre-amplified with the non-target primer pool, as a complex background for OA qPCR assays.

Strategy.

EGIR-harvested RNA from human normal skin was pre-amplified by 14 cycles of PCR in separate reactions with the TaqMan “target” and “non-target” primer pools. qPCR analyses were performed on the 7900HT system (ABI, Inc.) on each of 2 reaction pools for expression of each of the 20 target genes.

Purpose 2: Characterize sensitivity, linear dynamic range and reproducibility of the OA platform.

Strategy.

The above described Latin Square experiment was performed, targeting 20 genes in a complex background.

Purpose 3: Comparatively assess the performance of a multi-gene biomarker for melanoma on the TaqMan OA and microarray platforms.

Strategy.

Develop class prediction models for the microarray and OA platforms

TreeNet algorithm was applied to training dataset expression data, generated with each platform, for each of the 20 target genes. The test dataset consisted of 31 melanoma in situs and 46 invasive melanomas.

### Example 4

### Identification of Melanoma Molecular Signature

The study protocols were reviewed and approved by the various Institutional Review Boards that submit specimens for the study. Study subjects provided written consent prior to participation. The study was conducted according to the declaration of the Helsinki principles.

Inclusion Criteria.

Subjects were eligible and are recruited into the study if they were at least 18 years of age and had a pigmented lesion of at least 4 mms in diameter that is clinically suspicious for melanoma. All patients undergo a standard of care biopsy with histologic evaluation. If the patient has multiple clinically suspicious lesions, the lesions have to be at least 4 mms apart to be included in the study.

Exclusion Criteria.

Subjects cannot participate in the study if they have used topical medications (corticosteroids, alpha-hydroxyacids, retinoids, antibiotics) or systemic steroids within 30 days of beginning the research study or if the subject has a generalized skin disorder not related to skin cancer such as psoriasis, a photosensitivity dermatitis or eczema; or allergy to tape or latex rubber. Subjects are also excluded if they are currently participating, or have participated in the 30 days prior to the study, in an investigational (OTC, RX or device) study; or have clinical findings that the Investigator believes are suggestive of an advanced stage lesion (e.g. ulcerated, bleeding, oozing).

Lesion Selection Criteria.

Lesions suspicious for melanoma and selected for biopsy were tape striped prior to biopsy. Advanced stage lesions (e.g. ulcerated, bleeding, oozing) that may be physically damaged by the tape stripping procedure were not included.

Tape-Stripping Procedure.

Tape stripping was performed before all biopsy procedures. The tape was applied to the lesion site and briskly rubbed with the blunt rounded end of a marker or plastic test tube in a circular motion. A minimum of 15 circular motions were completed before the tape was removed. The border of the lesion was demarcated on the tape with a surgical marker. A total of 4 tapes were used to sample each site. After sampling, the tapes were stored at −20° C. or below within 10 minutes of stripping. The tapes were shipped on dry ice, by express mail, to be analyzed within one week of tape stripping.

Biopsy.

After the tape-stripping procedure was completed, the lesion was biopsied or excised according to standard clinical practice. The biopsy is a standard of care procedure that would have to be conducted regardless of the research. All tissues removed were fixed in formalin and sent to a histopathology laboratory, where they were embedded in paraffin and sectioned for histopathological analysis. Selected tissue slides (either the actual slides used for diagnosis or diagnostically equivalent slides) were sent to a dermatopathologist for central review. Three separate dermatopathologists were involved in the reading of each case. Further inclusion criteria for the study required histologic agreement between all three dermatopathologists involved in the study.

Quantitation of Gene Expression.

The area of the pigmented lesion is delineated on each piece of tape by the collecting site, and shipped on dry ice to a laboratory via overnight courier. The outlined lesional portion of each tape is manually dissected and the four tapes for each lesion are pooled for further processing. Total RNA is isolated for each sampled pigmented lesion and used for cDNA synthesis. cDNA is subsequently used for pre-amplification and real-time quantitative PCR (RT-qPCR) using standard Taqman chemistry. RNA is isolated using Ambion MELT™ (Life Technologies, Foster City Calif.); cDNA synthesis is performed using SuperScript VILO™ (Life Technologies); pre-amplification is performed using a custom pool of primers and probes for all genes assayed (Life Technologies). Quantitation of gene expression levels is performed using the QuantStudio OpenArray™ system (Life Technologies). Expression levels of signature genes for each specimen were normalized to the geometric mean of expression levels of housekeeping genes for that specimen. The gene signature comprised WFDC3, SLC16A6, DUSP4, PPAP2A, NDUFAF4, BCL2A1, DMXL2, SMG1, SLC2A3, VEGFA, BEX4, SH3BP5, RAB27A, FEM1A, CYTH3, IRGQ, EFNA5, NAMPT, PNPT1, and HNRNPH.

Statistical Analysis.

An optimal classification signature was created separately for each ascertained datasets. The selection of genes for inclusion in the classification signature was performed using stochastic gradient boosting analysis coupled with bootstrap logistic decision tree modeling (TreeNet™, Salford Systems, San Diego Calif.) and logistic regression (R: A Language and Environment for Statistical Computing. R Development Core Team, R Foundation for Statistical Computing. Vienna, Austria. 2011.) on the gene molecular signature list of Table 1. Bootstrapping is a machine learning technique that iteratively divides the dataset into random training and test classes and assesses classification performance for each iteration. TreeNet was used to create the classification algorithm and to determine each gene's relative importance within the signature. The classification algorithm computes a score between 0 and 1 for each specimen based on the measured gene expression inputs. Sensitivity, specificity, NPV, PPV, Receiver Operator Characteristic (ROC) curves, and AUC (area under the ROC curve) were calculated using R.

Results.

Gene Expression Classification of Melanoma—Melanoma In Situ Versus Invasive Melanoma: A high accuracy gene classification signature was used as a starting point for validation (see Example 3). In order to further understand the signature, the relative importance of each gene within the total profile was assessed.

All combinations from within the previous 20 gene signature were investigated for accuracy by bootstrap multivariate logistic regression using a specimen set, including 31 melanoma in situs and 46 invasive melanomas. The mean AUC of each combinations iterations were calculated. Gene pairs with average AUCs above 0.90 were further investigated using TreeNet analysis.

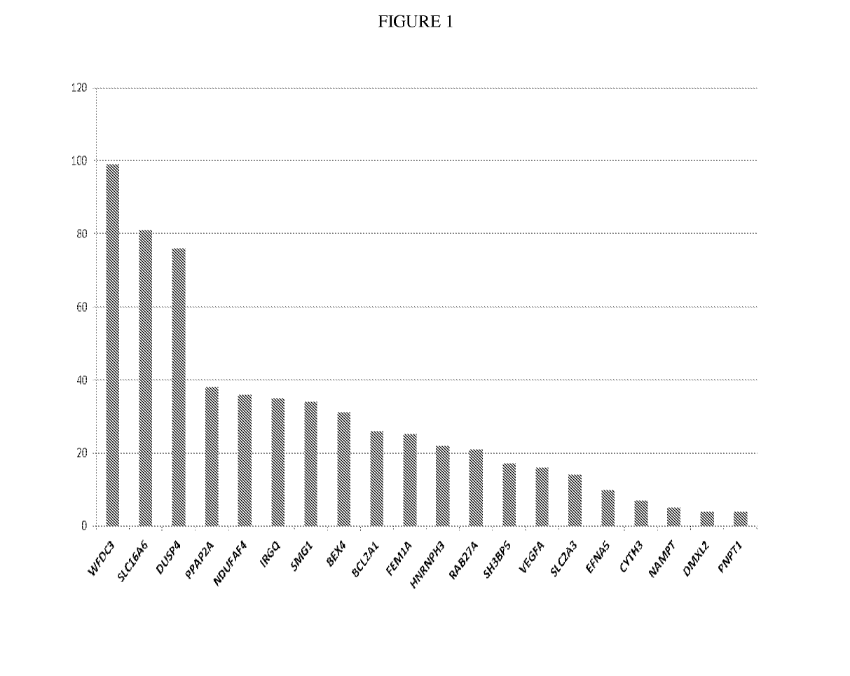

Logistic regression analyses of the 20-gene signature from Table 1 revealed that a classifier based on only 6 of the 20 genes has equivalent power to discriminate melanoma in situ from invasive melanoma. From the 38,760 possible combinations of 6 genes from the set of 20 genes used for initial classier creation, only 134 combinations had an AUC-ROC greater than 0.90 (less than 0.4% of all combinations). The frequency of the individual genes' occurrences among the 6-gene combinations is shown in FIG. 1. WFDC3 was present in almost 100 of the 6-gene combinations having AUC-ROC values greater than 0.90 for distinguishing between melanoma in situ and invasive melanoma. SLC16A6 was present in over 80 of the 6-gene combinations having AUC-ROC values greater than 0.90. DUSP4 was present in over 70 of the 6-gene combinations having AUC-ROC values greater than 0.90.

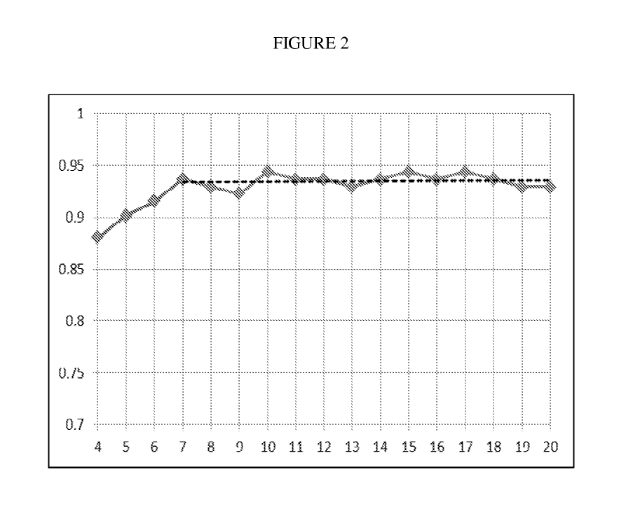

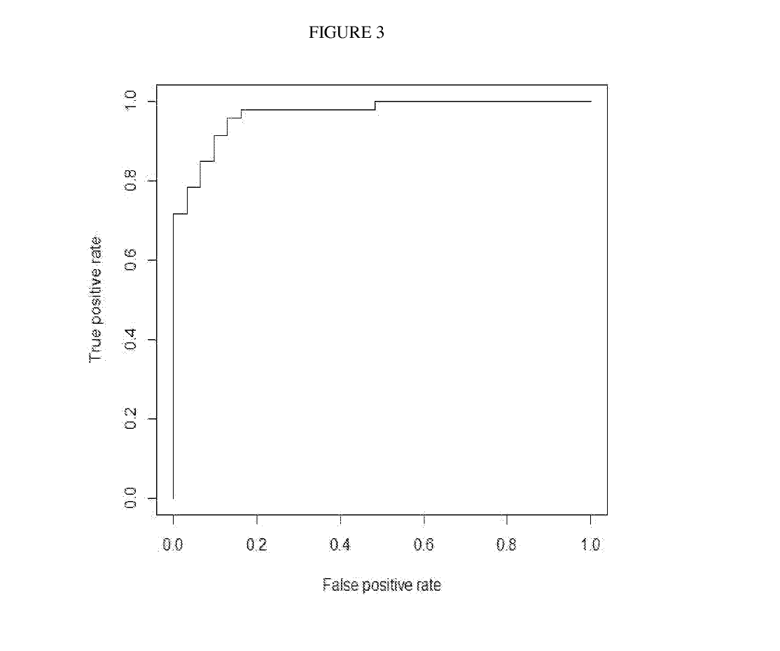

FIG. 2 is a plot of the average AUC-ROC calculated for each combination of genes from the 20-gene molecular signature of Table 1. FIG. 2 illustrates that classifiers comprising 7 of the genes from the 20-gene classifier have similar AUC-ROC values, on average, for distinguishing melanoma in situ from invasive melanoma as classifiers having, on average, 8 to 20 genes. The AUC-ROC values plotted in FIG. 2 are average values for all different combinations of the 20-gene classifier, and as such, some combinations had higher or lower AUC-ROC values. The best-performing 6-gene combination by logistic regression in this experiment comprises WFDC3, SLC16A6, DUSP4, PPAP2A, NDUFAF4, and BCL2A1. The ROC curve for this combination over all 77 samples has an AUC=0.968 (FIG. 3).

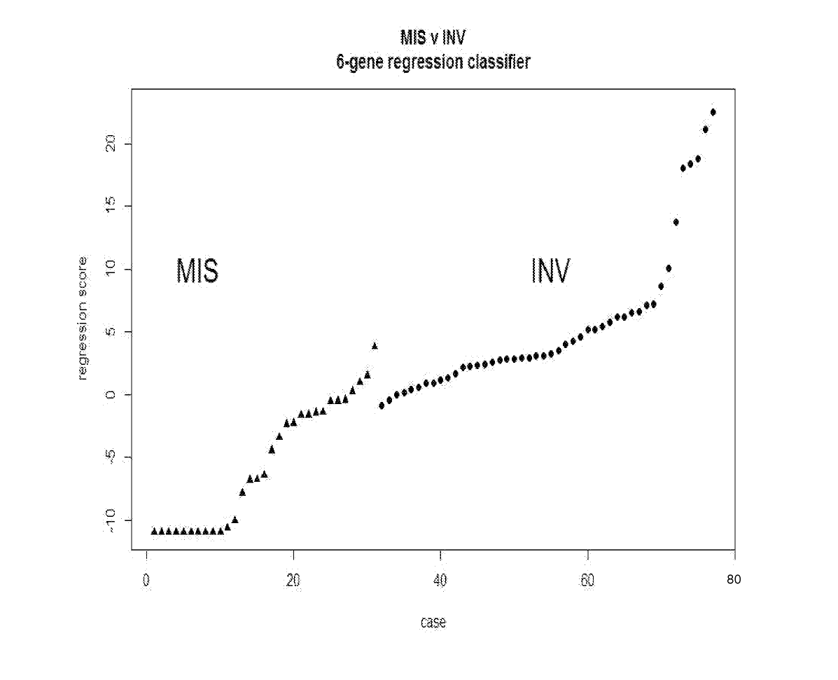

The 6-gene classifier (WFDC3, SLC16A6, DUSP4, PPAP2A, NDUFAF4, BCL2A1) score distribution for all 77 cases (31 melanoma in situs and 46 invasive melanomas) is shown in FIG. 4. The y-axis is the 6-gene classifier score, and the x-axis is the case. Melanoma in situ cases are represented by closed triangles and invasive melanoma cases are represented by closed circles. Using a threshold at −1, the sensitivity for classifying a sample as invasive melanoma is 100% (46 out 46 of the invasive melanomas were identified) and the specificity is 77% (24 out of the 31 melanoma in situs identified). It should be noted that the threshold value may be selected to trade-off between sensitivity and specificity.

In vivo, the mRNA from the critical signature genes produced by melanocytes is phagocytized by epithelial cells. The tape stripping method is non-invasive only removing a superficial layer of epithelial cells and does not result in any wound or require any down time from physical activity or work for the patient. While the overall morbidity associated with skin biopsy is quite low, each biopsy may result in decreased physical activity including time off from work, anxiety associated with having a procedure done and considerable cost.

Assuming an NNT (“number needed to treat”, used to measure physician accuracy in assessing pigmented skin lesions) between 10 and 30 for pigmented lesions, a single patient with 2 melanomas in their lifetime may have 20 to 60 biopsies to identify the 2 malignant neoplasms. Furthermore, some pigmented lesions on the face, genitalia or other sensitive anatomic locations may be difficult to biopsy and may result in unsightly scars. A high sensitivity of the assay (100%) accompanied with a specificity of 77%, could significantly decrease the number of unnecessary biopsies, therefore decreasing the NNT to a value of 2. This would result in a significant improvement in quality of life particularly for dysplastic nevus syndrome patients who may have hundreds of clinically atypical nevi and more than one melanoma in their lifetime. This could also result in a significant savings in health care expenditures.

While preferred embodiments of the present inventions have been shown and described herein, it will be obvious to those skilled in the art that such embodiments are provided by way of example only. Numerous variations, changes, and substitutions will now occur to those skilled in the art without departing from the inventions. It should be understood that various alternatives to the embodiments of the inventions described herein may be employed in practicing the inventions. It is intended that the following claims define the scope of the inventions and that methods and structures within the scope of these claims and their equivalents be covered thereby.

1-110. (canceled)

111. A method of distinguishing melanoma in situ from invasive melanoma in a subject in need thereof, comprising:
a) analyzing a biological sample obtained from the subject to generate an expression profile of one or more genes of a gene classifier comprising WFDC3, SLC16A6, DUSP4, PPAP2A, NDUFAF4 and BCL2A1 by applying one or more probes to gene expression products of one or more of the genes of the gene classifier in the biological sample, wherein the one or more probes comprise nucleic acid sequences specific and complementary to one or more of the genes of the gene classifier, and hybridizing the one or more probes to the gene expression products; and
b) classifying the subject as having melanoma in situ or invasive melanoma based on the expression profile, thereby distinguishing melanoma in situ from invasive melanoma.

112. The method of claim 111, wherein classifying in step b) further comprises comparing the expression profile of the one or more genes of the gene classifier in the biological sample to a gene expression profile of the one or more genes of the gene classifier in at least one reference sample, provided that the at least one reference sample is selected from an individual with melanoma in situ or invasive melanoma.

113. The method of claim 111, further comprising determining an expression profile for at least one additional gene selected from DMXL2, SMG1, SLC2A3, VEGFA, BEX4, SH3BP5, RAB27A, FEM1A, CYTH3, IRGQ, EFNA5, NAMPT, PNPT1, and HNRNPH3.

114. The method of claim 111, provided that the biological sample is a skin sample.

115. The method of claim 114, provided that the skin sample is a skin lesion sample.

116. The method of claim 114, provided that the skin sample is obtained by applying an adhesive tape to a target area of skin in a manner sufficient to adhere the skin sample to the adhesive tape and removing the adhesive tape from the skin in a manner sufficient to retain the adhered skin sample to the adhesive tape.

117. The method of claim 111, provided that the gene expression products are cDNA molecules reversely transcribed from RNA molecules expressed from the one or more genes of the gene classifier.

118. The method of claim 111, wherein the analyzing comprises microarray analysis, digital gene expression, a direct sequencing method or polymerase chain reaction (PCR).

119. The method of claim 111, further comprising providing an appropriate treatment option.

120. The method of claim 119, wherein the appropriate treatment option comprises biopsy, chemotherapeutic treatment, radiation treatment, anti-angiogenic compounds, or combinations thereof.

121. A kit for distinguishing melanoma in situ from invasive melanoma in a subject in need thereof, comprising at least one primer or probe for the detection of gene expression products of one or more genes of a gene classifier comprising WFDC3, SLC16A6, DUSP4, PPAP2A, NDUFAF4 and BCL2A1.

122. The kit of claim 121, wherein the gene expression products are nucleic acid molecules or protein molecules.

123. The kit of claim 122, wherein the nucleic acid molecules are RNA molecules.

124. The kit of claim 123, wherein the at least one primer or probe hybridizes to one or more cDNA molecules transcribed from the RNA molecules.

125. The kit of claim 121, further comprising a skin sample collection device and/or a sample collector.

126. The kit of claim 125, provided that the skin sample collection device is an adhesive tape.

127. A method of distinguishing melanoma in situ from invasive melanoma in a subject in need thereof and administering a treatment option to the subject, comprising:
a) analyzing a biological sample obtained from the subject to generate an expression profile of one or more genes of a gene classifier comprising WFDC3, SLC16A6, DUSP4, PPAP2A, NDUFAF4 and BCL2A1;
b) classifying the subject as having melanoma in situ or invasive melanoma based on the expression profile, thereby distinguishing melanoma in situ from invasive melanoma; and
c) based on step b), administering an appropriate treatment option to the subject, wherein the appropriate treatment option is a treatment for melanoma in situ or a treatment for invasive melanoma.

128. The method of claim 127, wherein classifying in step b) further comprises comparing the expression profile of the one or more genes of the gene classifier in the biological sample to a gene expression profile of the one or more genes of the gene classifier in at least one reference sample, provided that the at least one reference sample is selected from an individual with melanoma in situ or invasive melanoma.

129. The method of claim 127, wherein the analyzing further comprises:
a) applying one or more probes to gene expression products expressed from the one or more genes of the gene classifier; provided that the one or more probes comprise nucleic acid sequences specific and complementary to one or more of the genes of the gene classifier; and
b) hybridizing the one or more probes to the gene expression products.

130. The method of claim 127, wherein the analyzing further comprises microarray analysis, digital gene expression, a direct sequencing method or polymerase chain reaction (PCR).
